(19) United States
(12) Patent Application Publication
(10) Pub. No.: US 2025/0270301 A1
(43) Pub. Date: Aug. 28, 2025

(54) TAU BINDING MOLECULES

(71) Applicant: Gen2 Neuroscience Limited, Manchester (GB)

(72) Inventors: Frederick John Livesey, Manchester (GB); Clare Jones, Cambridge (GB)

(73) Assignee: Gen2 Neuroscience Limited, Manchester (GB)

(21) Appl. No.: 18/003,731

(22) PCT Filed: Jun. 29, 2021
(86) PCT No.: PCT/EP2021/067929

§ 371 Date: Dec. 29, 2022

(30) Foreign Application Priority Data
Jun. 29, 2020 (GB) 2009930.5
Jun. 29, 2020 (WO) PCT/EP2020/068314
Dec. 30, 2020 (GB) 2020754.4

(51) Int. Cl. C07K 16/18; A61K 39/00; A61P 25/28; G01N 33/68

(52) U.S. Cl. CPC C07K 16/18; A61P 25/28; G01N 33/6896; A61K 2039/505; C07K 2317/24; C07K 2317/565; C07K 2317/567; C07K 2317/732; C07K 2317/92; G01N 2333/4703

(57) ABSTRACT

The invention relates to isolated recombinant peptides comprising an epitope from human tau 2N4R. The invention also relates to antibodies, specific for isolated recombinant peptides comprising an epitope from human tau 2N4R and to such antibodies for use in investigation, diagnosis and treatment of tauopathies, such as Alzheimer's disease.

### SEQUENCE LISTING

The instant application contains a Sequence Listing which has been submitted electronically in ASCII format via the Patent Center and hereby incorporated by reference in its entirety. Said ASCII copy, created on 18 Dec. 2024, is named GEN2BH001US1_Corr_ST25.txt and is 111,446 bytes in size.

### FIELD OF THE INVENTION

The invention relates to binding molecules, such as antibodies, capable of binding specifically to novel tau epitopes. The invention relates to anti-tau binding molecules, such as antibodies, and compositions thereof, for use in the treatment or diagnosis of a tauopathy. The invention further relates to methods of treating a tauopathy, involving administering an anti-tau binding molecule, e.g., antibody.

### BACKGROUND TO THE INVENTION

The microtubule-associated protein (MAP) tau plays a critical role in the pathogenesis of Alzheimer's disease (AD) and related tauopathies. Development of tau pathology is associated with progressive neuronal loss and cognitive decline. In patients with dementias that involve tau, including Alzheimer's disease (AD), tau pathology spreads through the brain in a predictable spatial order, which correlates with disease burden. Recent evidence suggests the involvement of extracellular tau species in the propagation between neurons of neurofibrillary lesions and the spread of tau toxicity throughout different brain regions. The mechanism underlying tau propagation is not fully characterised, but suggests a role for extracellular tau in both cognitive decline and in the spreading of tau pathology, through synaptic and non-synaptic mechanisms.

Tau proteins are produced by alternative splicing from a single gene, MAPT (microtubule-associated protein tau); in humans the MAPT gene is located on chromosome 17q21. Tau proteins are abundant in neurons of the central nervous system and are also expressed at very low levels in CNS astrocytes and oligodendrocytes. Within neurons, tau is found predominantly in axons as a highly soluble phosphoprotein. Tau is post-translationally modified, with both physiological and pathophysiological consequences. Acetylation, ubiquitination, O-linked N-acetylglucosamine modification, methylation and phosphorylation of tau have all been described to regulate the function of tau (Morris et al (2015) Nature Neuroscience 18:1183-1189). In addition, tau may be cleaved to form peptides with enhanced ability to form aggregates and/or with neurotoxic properties.

The microtubule-associated protein tau and its hyperphosphorylated version form the main constituent of intracellular neurofibrillary tangles, a hallmark of several dementias, including AD and frontotemporal dementia. This evidence forms the basis of a hypothesis for AD, wherein the intracellular accumulation of tau leads to microtubule disassembly, dendritic spinal collapse, and degeneration of axons; malfunction in communication between neurons and cell death. Accordingly, tau, particularly in phosphorylated form, has been the target for development of passive and active immunotherapies for AD and other tauopathies.

Immunotherapies in clinical development that target various epitopes of tau were summarized by Pedersen et al. (2015) Trends Mol Med 21 (6): 394-402:

| Antibody | | | | |
|---|---|---|---|---|
| program | Antibody | Species | Epitope | IP |
| ACImmune, | ACI-35 | Active vaccine | pS396/pS404 peptide | WO2010115843 |
| Janssen, KU | | | liposome formulation | |
| Leuven, and | | | | |
| Fred van | | | | |
| Leuven | | | | |
| ACImmune, | hACI-36- | Humanized | pS409 | WO2013151762 |
| Genentech, KU | 2B6-Ab1 | mouse | | |
| Leuven, and | and | monoclonal | | |
| Fred van | hACI-36- | | | |
| Leuven | 3A8-Ab1 | | | |
| Axon | AADvac1 | Active vaccine | 294-305 peptide | WO02062851 |
| Neuroscience | | | 294KDNIKHVPGGGS305 | |
| and Michal | | | | |
| Novak | | | | |
| Axon | DC8E8 | Humanized | Generated from | WO02062851 |
| Neuroscience | | antibody | immunizations with | |
| and Michal | | | 294-305 peptide | |
| Novak | | | | |
| Biogen Idec, | NI- | Human | V339, E342, D387, | WO2012049570 |
| Panima | 105.4E4, | autoantibodies | E391, K395 | US2012087861 |
| Pharmaceuticals | 24B2, | | | |
| AG, and | and 4A3 | | | |
| Roger Nitsch | | | | |
| C2N | HJ9.3, | Mouse | 306-320, 7-13, and | |
| Diagnostics, | HJ9.4, | monoclonal | 25-30 | |
| David | and | | | |
| Holtzman, and | HJ8.5 | | | |
| Marc Diamond | | | | |
| Eli Lilly and | PHF1, | Mouse | pS396/pS404, | WO9620218 |
| Peter Davies | MC1 | monoclonal | conformational | |
| Hoffman-La | 2.10.2, | Rabbit | pS422 | WO2010142423 |
| Roche | 2.20.4, | monoclonal | | |
| | and | | | |
| | 5.6.11 | | | |
| | MAb86 | | | |
| iPierian/BMS | IPN001, | Humanized | 9-18 | US2014294831 |
| | IPN002, | mouse | | |
| | IPN007 | monoclonal | | |
| Intellect | TOC-1 | Mouse | Tau-dimers and | US8697076 |
| Neurosciences | and | monoclonal | caspase-cleaved | US2012244174 |
| Inc. and Lester | TauC3 | | Tau421 | |
| Binder | | | | |
| Lundbeck, | 4E6, 6B2, | Mouse | pS396/pS404, total tau, | US2008050383 |
| NYU, and Einar | and | monoclonal | other | US2010316564 |
| Sigurdsson | scFv235 | | hyperphosphorylation, | |
| | | | conformational, and | |
| | | | truncation sites | |
| Pfizer | | Chicken | pT212/pS214, | WO2014016737 |
| | | monoclonal | pT231/pS235, and | |
| | | | pS396/pS404 | |
| Prothena | pS404- | Mouse | pS404 and total tau | WO2014134685 |
| Corporation, | Ab1/Ab2 | monoclonal | | |
| Lars Ittner, and | pan-tau | | | |
| Jurgen Gotz | | | | |
| Prothena | h16B5 | Humanized | 23-46 peptide | WO2014165271 |
| Corporation | | mouse | | |
| | | monoclonal | | |
| Teijin Pharma | Ta1505 | Mouse | pSer413 | WO2013180238 |
| Ltd and Hitoshi | | monoclonal | | |
| Mori | | | | |

### Table 1: Immunotherapies in Clinical Development

In addition to therapies listed in Table 1, Janssen are progressing antibodies specifically targeting pT217 (JNJ63733657) and UCB are in the clinic with antibodies targeting a mid-region tau sequence (amino acids 235-246 of 2N4R tau; UCB0107) (reviewed in Sandusky-Beltran et al., 2020, Neuropharmacol. 175:108104). Eisai are also preparing for clinical trials with an antibody targeting sequences in the microtubule binding region (amino acids 299-303 and 362-366; E2814; Roberts et al., 2020, Acta Neuropathologica Comms 8:13).

U.S. Pat. No. 9,139,643B2 describes an antibody, specific for misfolded and/or aggregated tau protein that does not bind to normal tau protein and which binds an epitope within amino acid residues 379-408 of full length human 2N4R (amino acids 1-441) tau (SEQ ID NO:2), it is preferred that the tau protein is fully phosphorylated.

U.S. Pat. No. 9,777,056B2 describes an antibody, capable of binding specifically to a misfolded and/or aggregate form of tau protein, raised against a tau epitope within amino acid residues 379-408 that possesses phosphoserine residues at tau position 396 and at tau position 404.

WO2010144711 describes recombinantly-produced antibodies capable of preferentially binding to pathological tau protein, relative to normal tau protein, elicited by immunization with various isolated tau peptides including tau 379-408, SEQ ID NO: 57 of that specification, and tau 379-391, SEQ ID NO: 102 of that specification.

US 2017/0260263 A1 describes tau peptides comprising the “therapeutic epitope” Tau 361-THVPGGG-367 (SEQ ID NO: 101 of that specification).

US2004/0110250 A1 describes tau aggregation and regions important for the assembly of PHF tau. It does not mention tau uptake, or therapeutic antibodies. Tau 186-391 constructs are expressed intracellularly, proteolytically processed intracellularly and the resulting truncated peptides detected intracellularly. There is no discussion of extracellular forms of tau.

WO 2011/032155 A2 discloses fragment-specific antibodies that target epitopes generated when tau is cleaved by calpain and that do not bind full-length human Tau441 protein.

WO 2018/178077 A1 discloses a monoclonal antibody that binds to an epitope comprising the amino acid residues 299-318 of the human Tau441 protein.

WO 2014/159244 discloses an antibody specific for binding the O-GlcNAcylated tau isoform 2N4R at serine 400.

Recent studies suggest a toxic role for disease-specific tau species located in the brain extracellular space.

There is a need to identify novel therapeutic approaches intended to interfere early in the process of tau-mediated synaptic dysfunction and the propagation of tau pathology; accordingly, there is a need to identify epitopes of tau that occur specifically on extracellular pathological species of the tau protein and to generate therapeutic antibodies that seek to halt disease progression by binding specifically to extracellular pathological species of the tau protein. Such epitopes and molecules that bind thereto may also be useful for diagnosis of tauopathies.

### Statements of Invention

The invention provides:

1. A humanised antibody or antigen-binding fragment thereof, capable of binding specifically to an epitope formed by residues of the amino acid sequence 369-381 (SEQ ID NO: 1) of human 2N4R (amino acids 1-441) tau (SEQ ID NO: 2).

2. A humanised antibody or antigen-binding fragment thereof, according to clause 1, capable of binding specifically to an epitope formed by residues of the amino acid sequence 373 to 379 (THKLTFR, SEQ ID NO: 150) of human 2N4R (amino acids 1-441) tau (SEQ ID NO: 2), preferably wherein the epitope comprises residues: K375, T377 and R379, more preferably wherein the epitope comprises residues T373, K375, T377 and R379.

3. A humanised antibody or antigen-binding fragment thereof, of clause 1 or clause 2 comprising an antigen-binding site comprising human framework sequences (FW1 to FW4) and CDRs (HCDR1, HCRD2, HCDR3, LCDR1, LCDR2 and LCDR3, respectively) wherein HCDR1 is SEQ ID NO: 20; HCDR2 is SEQ ID NO: 21 or a variant wherein: amino acid 51 is selected from C, V and A; amino acid 54 is selected from R, A and S; amino acid 55 is selected from R, A and V; and amino acid 57 is selected from G, H, N, R, A and S; HCDR3 is SEQ ID NO: 22 or a variant wherein: amino acid 96 is selected from S, V, R and A; amino acid 98 is selected from A, S, D, H and T; amino acid 102 is selected from P, V and Y; LCDR1 is SEQ ID NO: 23, LCDR2 is SEQ ID NO: 24 and LCDR3 is selected from SEQ ID NO: 25 or SEQ ID NO 207.

4. A humanised antibody or antigen-binding fragment thereof, of any preceding clause, comprising an antigen-binding site comprising human framework sequences (FW1 to FW4) and CDRs (HCDR1, HCRD2, HCDR3, LCDR1, LCDR2 and LCDR3, respectively) selected from:
(a) SEQ ID NO: 20, SEQ ID NO: 21, SEQ ID NO: 164, SEQ ID NO: 23, SEQ ID NO: 24 and SEQ ID NO: 25 (VH4_H04);
(b) SEQ ID NO: 20, SEQ ID NO: 21, SEQ ID NO: 162, SEQ ID NO: 23, SEQ ID NO: 24 and SEQ ID NO: 25 (VH4_H02);
(c) SEQ ID NO: 20, SEQ ID NO: 167, SEQ ID NO: 166, SEQ ID NO: 23, SEQ ID NO: 24 and SEQ ID NO: 25 (VH4_H06);
(d) SEQ ID NO: 20, SEQ ID NO: 21, SEQ ID NO: 161, SEQ ID NO: 23, SEQ ID NO: 24 and SEQ ID NO: 25 (VH4_H01);
(e) SEQ ID NO: 20, SEQ ID NO: 167, SEQ ID NO: 164, SEQ ID NO: 23, SEQ ID NO: 24 and SEQ ID NO: 25 (VH4_H06_H04);
(f) SEQ ID NO: 20, SEQ ID NO: 21, SEQ ID NO: 22, SEQ ID NO: 23, SEQ ID NO: 24 and SEQ ID NO: 25 (VH4VK4);
(g) SEQ ID NO: 20, SEQ ID NO: 21, SEQ ID NO: 163, SEQ ID NO: 23, SEQ ID NO: 24 and SEQ ID NO: 25 (VH4_H03)
(h) SEQ ID NO: 20, SEQ ID NO: 21, SEQ ID NO: 165, SEQ ID NO: 23, SEQ ID NO: 24 and SEQ ID NO: 25 (VH4_H05);
(i) SEQ ID NO: 20, SEQ ID NO: 168, SEQ ID NO: 22, SEQ ID NO: 23, SEQ ID NO: 24 and SEQ ID NO: 25 (VH4_H07);
(j) SEQ ID NO: 20, SEQ ID NO: 169, SEQ ID NO: 22, SEQ ID NO: 23, SEQ ID NO: 24 and SEQ ID NO: 25 (VH4_H08);
(k) SEQ ID NO: 20, SEQ ID NO: 170, SEQ ID NO: 22, SEQ ID NO: 23, SEQ ID NO: 24 and SEQ ID NO: 25 (VH4_H09);
(l) SEQ ID NO: 20, SEQ ID NO: 171, SEQ ID NO: 22, SEQ ID NO: 23, SEQ ID NO: 24 and SEQ ID NO: 25 (VH4_H10);
(m) SEQ ID NO: 20, SEQ ID NO: 172, SEQ ID NO: 22, SEQ ID NO: 23, SEQ ID NO: 24 and SEQ ID NO: 25 (VH4_H11);
(n) SEQ ID NO: 20, SEQ ID NO: 173, SEQ ID NO: 22, SEQ ID NO: 23, SEQ ID NO: 24 and SEQ ID NO: 25 (VH4_H12);
(o) SEQ ID NO: 20, SEQ ID NO: 174, SEQ ID NO: 22, SEQ ID NO: 23, SEQ ID NO: 24 and SEQ ID NO: 25 (VH4_H13);
(p) SEQ ID NO: 20, SEQ ID NO: 175, SEQ ID NO: 22, SEQ ID NO: 23, SEQ ID NO: 24 and SEQ ID NO: 25 (VH4_H14);
(q) SEQ ID NO: 20, SEQ ID NO: 176, SEQ ID NO: 22, SEQ ID NO: 23, SEQ ID NO: 24 and SEQ ID NO: 25 (VH4_H15);
(r) SEQ ID NO: 20, SEQ ID NO: 177, SEQ ID NO: 22, SEQ ID NO: 23, SEQ ID NO: 24 and SEQ ID NO: 25 (VH4_H16);
(s) SEQ ID NO: 20, SEQ ID NO: 178, SEQ ID NO: 22, SEQ ID NO: 23, SEQ ID NO: 24 and SEQ ID NO: 25 (VH4_H17);
(t) SEQ ID NO: 20, SEQ ID NO: 179, SEQ ID NO: 22, SEQ ID NO: 23, SEQ ID NO: 24 and SEQ ID NO: 25 (VH4_H18);
(u) SEQ ID NO: 20, SEQ ID NO: 180, SEQ ID NO: 22, SEQ ID NO: 23, SEQ ID NO: 24 and SEQ ID NO: 25 (VH4_H19);
(v) SEQ ID NO: 20, SEQ ID NO: 181, SEQ ID NO: 22, SEQ ID NO: 23, SEQ ID NO: 24 and SEQ ID NO: 25 (VH4_H20);
(w) SEQ ID NO: 20, SEQ ID NO: 167, SEQ ID NO: 161, SEQ ID NO: 23, SEQ ID NO: 24 and SEQ ID NO: 25 (VH4H_06_H01);
(x) SEQ ID NO: 20, SEQ ID NO: 167, SEQ ID NO: 162, SEQ ID NO: 23, SEQ ID NO: 24 and SEQ ID NO: 25 (VH4_H06_H02);
(y) SEQ ID NO: 20, SEQ ID NO: 177, SEQ ID NO: 164, SEQ ID NO: 23, SEQ ID NO: 24 and SEQ ID NO: 25 (VH4_H16_H04);
(z) SEQ ID NO: 20, SEQ ID NO: 21, SEQ ID NO: 22, SEQ ID NO: 23, SEQ ID NO: 24 and SEQ ID NO: 182 (VK4_L2);
(aa) SEQ ID NO: 20, SEQ ID NO: 167, SEQ ID NO: 166, SEQ ID NO: 23, SEQ ID NO: 24 and SEQ ID NO: 182 (VH4_H06/L2);
(bb) SEQ ID NO: 20, SEQ ID NO: 168, SEQ ID NO: 22, SEQ ID NO: 23, SEQ ID NO: 24 and SEQ ID NO: 182 (VH4_H07/L2);
(cc) SEQ ID NO: 20, SEQ ID NO: 170, SEQ ID NO: 22, SEQ ID NO: 23, SEQ ID NO: 24 and SEQ ID NO: 182 (VH4_H09/L2)
(dd) SEQ ID NO: 20, SEQ ID NO: 174, SEQ ID NO: 22, SEQ ID NO: 23, SEQ ID NO: 24 and SEQ ID NO: 182 (VH4_H13/L2); and
(ee) SEQ ID NO: 20, SEQ ID NO: 15, SEQ ID NO: 22, SEQ ID NO: 23, SEQ ID NO: 24 and SEQ ID NO: 182 (VH4_H14/L2);
wherein the sequences are defined according to Kabat nomenclature.

5. A humanised antibody or antigen-binding fragment thereof, of any preceding clause, wherein the antigen-binding site comprises the VH and/or VL domain sequence of, or a VH and/or VL domain sequence with at least 70, 75, 76, 77, 78, 79, 80, 81, 82, 83, 84, 85, 86, 87, 88, 89, 90, 91, 92, 93, 94, 95, 96, 97, 98 or 99% identity to, a clone selected from:
(a) Clone VH4_H04 of VH SEQ ID NO: 186 and VL SEQ ID NO: 160, respectively;
(b) Clone VH4_H02 of VH SEQ ID NO: 184 and VL SEQ ID NO: 160, respectively;
(c) Clone VH4_H06 of VH SEQ ID NO: 188 and VL SEQ ID NO: 160, respectively;
(d) Clone VH4_H01 of VH SEQ ID NO: 183 and VL SEQ ID NO: 160, respectively;
(e) Clone VH4_H06_H04 of VH SEQ ID NO: 205 and VL SEQ ID NO: 160, respectively;
(f) Clone VH4VK4 (E) of VH SEQ ID NO: 154 and VL SEQ ID NO: 160, respectively;
(g) Clone VH4_H03 of VH SEQ ID NO: 185 and VL SEQ ID NO: 160, respectively;
(h) Clone VH4_H05 of VH SEQ ID NO: 187 and VL SEQ ID NO: 160, respectively;
(i) Clone VH4_H07 of VH SEQ ID NO: 189 and VL SEQ ID NO: 160, respectively;
(j) Clone VH4_H08 of VH SEQ ID NO: 190 and VL SEQ ID NO: 160, respectively;
(k) Clone VH4_H09 of VH SEQ ID NO: 191 and VL SEQ ID NO: 160, respectively;
(l) Clone VH4_H10 of VH SEQ ID NO: 192 and VL SEQ ID NO: 160, respectively;
(m) Clone VH4_H11 of VH SEQ ID NO: 193 and VL SEQ ID NO: 160, respectively;
(n) Clone VH4_H12 of VH SEQ ID NO: 194 and VL SEQ ID NO: 160, respectively;
(o) Clone VH4_H13 of VH SEQ ID NO: 195 and VL SEQ ID NO: 160, respectively;
(p) Clone VH4_H14 of VH SEQ ID NO: 196 and VL SEQ ID NO: 160, respectively;
(q) Clone VH4_H15 of VH SEQ ID NO: 197 and VL SEQ ID NO: 160, respectively;
(r) Clone VH4_H16 of VH SEQ ID NO: 198 and VL SEQ ID NO: 160, respectively;
(s) Clone VH4_H17 of VH SEQ ID NO: 199 and VL SEQ ID NO: 160, respectively;
(t) Clone VH4_H18 of VH SEQ ID NO: 200 and VL SEQ ID NO: 160, respectively;
(u) Clone VH4_H19 of VH SEQ ID NO: 201 and VL SEQ ID NO: 160, respectively;
(v) Clone VH4_H20 of VH SEQ ID NO: 202 and VL SEQ ID NO: 160, respectively;
(w) Clone VH4H_06_H01 of VH SEQ ID NO: 203 and VL SEQ ID NO: 160, respectively;
(x) Clone VH4_H06_H02 of VH SEQ ID NO: 204 and VL SEQ ID NO: 160, respectively;
(y) Clone VH4_H16_H04 of VH SEQ ID NO: 206 and VL SEQ ID NO: 160, respectively;
(z) Clone VK4_L2 of VH SEQ ID NO: 154 and VL SEQ ID NO: 207, respectively;
(aa) Clone VH4_H06/L2 of VH SEQ ID NO: 188 and VL SEQ ID NO: 207, respectively;
(bb) Clone VH4_H07/L2 of VH SEQ ID NO: 189 and VL SEQ ID NO: 207, respectively;
(cc) Clone VH4_H09/L2 of VH SEQ ID NO: 191 and VL SEQ ID NO: 207, respectively
(dd) Clone VH4_H13/L2 of VH SEQ ID NO: 195 and VL SEQ ID NO: 207, respectively;
(ee) Clone VH4_H14/L2 of VH SEQ ID NO: 196 and VL SEQ ID NO: 207, respectively;
(ff) Clone VH3VK3 (A) of SEQ ID NO: 153 and SEQ ID NO: 159, respectively;
(gg) Clone VH3VK4 (B) of SEQ ID NO: 153 and SEQ ID NO: 160, respectively;
(hh) Clone VH4VK2 (C) of SEQ ID NO: 154 and SEQ ID NO: 158, respectively; and
(ii) Clone VH4VK3 (D) of SEQ ID NO: 154 and SEQ ID NO: 159, respectively;
wherein the sequences are defined according to Kabat nomenclature.

6. A humanised antibody or antigen-binding fragment thereof, of any preceding clause, wherein the antibody comprises the VH and/or VL domain of:
(a) Clone VH4_H04 of VH SEQ ID NO: 186 and VL SEQ ID NO: 160, respectively;
(b) Clone VH4_H02 of VH SEQ ID NO: 184 and VL SEQ ID NO: 160, respectively;
(c) Clone VH4_H06 of VH SEQ ID NO: 188 and VL SEQ ID NO: 160, respectively;
(d) Clone VH4_H01 of VH SEQ ID NO: 183 and VL SEQ ID NO: 160, respectively;
(e) Clone VH4_H06_H04 of VH SEQ ID NO: 205 and VL SEQ ID NO: 160, respectively;
(f) Clone VH4VK4 (E) of VH SEQ ID NO: 154 and VL SEQ ID NO: 160, respectively;
(g) Clone VH4_H03 of VH SEQ ID NO: 185 and VL SEQ ID NO: 160, respectively;
(h) Clone VH4_H05 of VH SEQ ID NO: 187 and VL SEQ ID NO: 160, respectively;
(i) Clone VH4_H07 of VH SEQ ID NO: 189 and VL SEQ ID NO: 160, respectively;
(j) Clone VH4_H08 of VH SEQ ID NO: 190 and VL SEQ ID NO: 160, respectively;
(k) Clone VH4_H09 of VH SEQ ID NO: 191 and VL SEQ ID NO: 160, respectively;
(l) Clone VH4_H10 of VH SEQ ID NO: 192 and VL SEQ ID NO: 160, respectively;
(m) Clone VH4_H11 of VH SEQ ID NO: 193 and VL SEQ ID NO: 160, respectively;
(n) Clone VH4_H12 of VH SEQ ID NO: 194 and VL SEQ ID NO: 160, respectively;
(o) Clone VH4_H13 of VH SEQ ID NO: 195 and VL SEQ ID NO: 160, respectively;
(p) Clone VH4_H14 of VH SEQ ID NO: 196 and VL SEQ ID NO: 160, respectively;
(q) Clone VH4_H15 of VH SEQ ID NO: 197 and VL SEQ ID NO: 160, respectively;
(r) Clone VH4_H16 of VH SEQ ID NO: 198 and VL SEQ ID NO: 160, respectively;
(s) Clone VH4_H17 of VH SEQ ID NO: 199 and VL SEQ ID NO: 160, respectively;
(t) Clone VH4_H18 of VH SEQ ID NO: 200 and VL SEQ ID NO: 160, respectively;
(u) Clone VH4_H19 of VH SEQ ID NO: 201 and VL SEQ ID NO: 160, respectively;
(v) Clone VH4_H20 of VH SEQ ID NO: 202 and VL SEQ ID NO: 160, respectively;
(w) Clone VH4H_06_H01 of VH SEQ ID NO: 203 and VL SEQ ID NO: 160, respectively;
(x) Clone VH4_H06_H02 of VH SEQ ID NO: 204 and VL SEQ ID NO: 160 respectively;
(y) Clone VH4_H16_H04 of VH SEQ ID NO: 206 and VL SEQ ID NO: 160, respectively;
(z) Clone VK4_L2 of VH SEQ ID NO: 154 and VL SEQ ID NO: 207, respectively;
(aa) Clone VH4_H06/L2 of VH SEQ ID NO: 188 and VL SEQ ID NO: 207, respectively;
(bb) Clone VH4_H07/L2 of VH SEQ ID NO: 189 and VL SEQ ID NO: 207, respectively;
(cc) Clone VH4_H09/L2 of VH SEQ ID NO: 191 and VL SEQ ID NO: 207, respectively
(dd) Clone VH4_H13/L2 of VH SEQ ID NO: 195 and VL SEQ ID NO: 207, respectively;
(ee) Clone VH4_H14/L2 of VH SEQ ID NO: 196 and VL SEQ ID NO: 207, respectively;
(ff) Clone VH3VK3 (A) of SEQ ID NO: 153 and SEQ ID NO: 159, respectively;
(gg) Clone VH3VK4 (B) of SEQ ID NO: 153 and SEQ ID NO: 160, respectively;
(hh) Clone VH4VK2 (C) of SEQ ID NO: 154 and SEQ ID NO: 158, respectively; and
(ii) Clone VH4VK3 (D) of SEQ ID NO: 154 and SEQ ID NO: 159, respectively;
wherein the sequences are defined according to Kabat nomenclature.

7. A humanised or human antibody or antigen-binding fragment thereof capable of competing with an antibody according to any one of clauses 1 to 6 for binding to an epitope formed by residues of the amino acid sequence 373 to 379 (THKLTFR, SEQ ID NO: 150) of human 2N4R (amino acids 1-441) tau (SEQ ID NO: 2) when assessed in a competition assay.

8. An isolated antibody or antigen-binding fragment thereof, of any preceding clause which is the product of expression of a recombinant DNA or RNA sequence.

9. An isolated recombinant DNA or RNA sequence comprising a sequence encoding an isolated antibody or antigen-binding fragment thereof, according to any one of clauses 1 to 8.

10. An isolated recombinant DNA sequence of clause 9 which is a vector.

11. An isolated recombinant DNA sequence of clause 10 which is an expression vector.

12. An isolated recombinant DNA sequence of clause 10 or 11 encoding an antibody or antigen-binding fragment thereof, according to any one of clauses 1 to 8 under control of a promoter.

13. A host cell comprising a DNA or RNA sequence according to any one of clauses 8 to 12.

14. A host cell of clause 13 capable of expressing an isolated antibody or antigen-binding fragment thereof, of any one of clauses 1 to 8.

15. A method of making an isolated antibody or antigen-binding fragment thereof, of any one of clauses 1 to 8 comprising culturing a host cell according to clause 13 or 14 in conditions suitable for expression of the isolated antibody or antigen-binding fragment thereof.

16. A composition comprising an isolated antibody or antigen-binding fragment thereof, according to any one of clauses 1 to 8 and a diluent, preferably a pharmaceutically acceptable diluent.

17. An antibody or antigen-binding fragment thereof of any one of clauses 1 to 8, or composition of clause 16 for use as a medicament or for use in diagnosis.

18. An antibody or antigen-binding fragment thereof any one of clauses 1 to 8, or a composition of clause 16, for use in the prophylactic or therapeutic treatment of a tauopathy, or for the manufacture of a medicament for the prophylactic or therapeutic treatment of a tauopathy, preferably the tauopathy is selected from Alzheimer's disease (sporadic and monogenic familial forms), amyotrophic lateral sclerosis/parkinsonism-dementia complex, argyrophilic grains disease, beta-propeller protein associated neurodegeneration (BPAN), British type amyloid angiopathy, cerebral amyloid angiopathy, Creutzfeldt-Jakob disease, dementia pugilistica, diffuse neurofibrillary tangles with calcification, Down's syndrome, chronic traumatic encephalopathy (CTE), corticobasal degeneration (CBD), frontotemporal dementia (FTD), frontotemporal dementia and parkinsonism linked to chromosome 17 (FTDP-17), frontotemporal lobar degeneration, Gerstmann-Straussler-Scheinker disease, Hallervorden-Spatz disease, inclusion body myositis, multiple system atrophy, myotonic dystrophy, Niemann-pick disease type C, non-guamanian motor neuron disease with neurofibrillary tangles, Parkinson's disease (sporadic and monogenic familial forms), Pick's disease, post-encephalitic parkinsonism, primary age-related tauopathy (PART), prion protein cerebral amyloid angiopathy, progressive subcortical gliosis, progressive supranuclear palsy (PSP), subacute sclerosing panencephalitis, tangle-dominant dementia, globular glial tauopathy, parkinsonism dementia complex of Guam, progressive non-fluent aphasia, multi-infarct dementia, ischemic stroke, traumatic brain injury (TBI) and stroke.

19. An antibody or antigen-binding fragment thereof of any one of clauses 1 to 8, or a composition of clause 16, that is capable of increasing phagocytosis of tau species in human microglia and/or reducing uptake of monomeric and aggregated tau species by human neurons and/or promoting uptake of tau species by human astrocytes and/or preventing uptake of tau species by human astrocytes and/or preventing tau-mediated inhibition of long term potentiation in rodent models.

20. An antibody or antigen-binding fragment thereof of any one of clauses 1 to 8, or a composition of clause 16, for use to identify human tau proteins comprising an epitope formed by residues 369-381 (SEQ ID NO: 1) of human 2N4R tau (SEQ ID NO: 2), preferably comprising an epitope formed by residues of the amino acid sequence 373 to 379 (THKLTFR, SEQ ID NO: 150) of human 2N4R (amino acids 1-441) tau (SEQ ID NO: 2).

21. An antibody or antigen-binding fragment thereof of any one of clauses 1 to 8, or a composition of clause 16, for use in a diagnostic test for a tauopathy.

22. A diagnostic kit comprising an antibody or antigen-binding fragment thereof of any one of clauses 1 to 8, or a composition of clause 16 and a reagent capable of detecting an immunological (antigen-antibody) complex which contains said isolated recombinant peptide binding molecule, antigen-binding protein or fragment thereof, wherein optionally said isolated recombinant peptide and/or binding molecule, antigen-binding protein or fragment thereof is immobilized on a solid support (e.g., microplate well), and/or wherein optionally said immunological complex which contains said isolated recombinant peptide, binding molecule, antigen-binding protein or fragment thereof is detectable by ELISA or an alternative immunoassay method or by lateral flow.

23. A diagnostic kit according to clause 22, further comprising one or more control standards and/or specimen diluent and/or washing buffer.

### DETAILED DESCRIPTION

The invention relates to humanised antibodies and antigen binding fragments thereof capable of binding specifically to an isolated recombinant peptide comprising an epitope formed within residues 369-381 (KKIETHKLTFREN, SEQ ID NO: 1) of human tau (tau1-441 SEQ ID NO: 2). The invention further relates to humanised antibodies and antigen binding fragments that comprise a CDR-based antigen-binding site, specific for an epitope comprised within residues 369-381 (KKIETHKLTFREN, SEQ ID NO: 1) of human tau (tau1-441 SEQ ID NO: 2).

Humanised antibodies and antigen binding fragments thereof of the invention bind specifically to extracellular tau species that include epitopes formed by residues 369-381 (KKIETHKLTFREN, SEQ ID NO: 1) of human tau (tau1-441 SEQ ID NO: 2).

Humanised antibodies and antigen binding fragments thereof of the invention are capable of binding specifically to an isolated recombinant peptide that further comprises a N-terminal cysteine (CKKIETHKLTFREN, SEQ ID NO: 13) or a C-terminal cysteine for conjugation of a carrier protein or detectable label. Carrier proteins that may be conjugated via the N-terminal cysteine may be selected from Keyhole limpet hemocyanin (KLH), Concholepas concholepas hemocyanin (“Blue Carrier”), Bovine serum albumin (BSA), Cationized BSA (cBSA) and Ovalbumin (OVA). Humanised antibodies and antigen binding fragments thereof of the invention are capable of binding specifically to conjugates comprising KKIETHKLTFREN, SEQ ID NO: 1.

An antibody or antigen-binding fragment thereof of the invention may be produced by recombinant means. A “recombinant antibody” is an antibody which has been produced by a recombinantly engineered host cell. An antibody or antigen-binding fragment thereof in accordance with the invention is optionally isolated or purified.

The term “antibody” or “antibody molecule” describes an immunoglobulin whether natural or partly or wholly synthetically produced. An antigen-binding protein of the invention may be an antibody, preferably a monoclonal antibody, and may be human or non-human, chimeric or humanised.

The antibody molecule is preferably a monoclonal antibody molecule. Examples of antibodies are the immunoglobulin isotypes, such as immunoglobulin G, and their isotypic subclasses, such as IgG1, IgG2, IgG3 and IgG4, as well as fragments thereof. The four human subclasses (IgG1, IgG2, IgG3 and IgG4) each contain a different heavy chain; but they are highly homologous and differ mainly in the hinge region and the extent to which they activate the host immune system. IgG1 and IgG4 contain two inter-chain disulphide bonds in the hinge region, IgG2 has 4 and IgG3 has 11 inter-chain disulphide bonds.

The terms “antibody” and “antibody molecule”, as used herein, includes antibody fragments, such as Fab and scFv fragments, provided that said fragments comprise a CDR-based antigen binding site for an epitope comprising residues 369-381 of human tau. Examples of antibody fragments include but are not limited to Fv, Fab, Fab′, Fab′-SH, F(ab′)2; diabodies; linear antibodies; single-chain antibody molecules (e.g., scFv) and domain antibodies (sdAbs). Unless the context requires otherwise, the terms “antigen-binding protein”, “antibody” or “antibody molecule”, as used herein, is thus equivalent to “antibody or antigen-binding fragment thereof”.

Antibodies are immunoglobulins, which have the same basic structure consisting of two heavy and two light chains forming two Fab arms containing identical domains that are attached by a flexible hinge region to the stem of the antibody, the Fc domain, giving the classical ‘Y’ shape. The Fab domains consist of two variable and two constant domains, with a variable heavy (VH) and constant heavy 1 (CH1) domain on the heavy chain and a variable light (VL) and constant light (CL) domain on the light chain. The two variable domains (VH and VL) form the variable fragment (Fv), which provides the CDR-based antigen specificity of the antibody, with the constant domains (CH1 and VL) acting as a structural framework. Each variable domain contains three hypervariable loops, known as complementarity determining regions (CDRs). On each of the VH and VL the three CDRs (CDR1, CDR2, and CDR3) are flanked by four less-variable framework (FR) regions (FR1, FW2, FW3 and FW4) to give a structure FW1-CDR1-FW2-CDR2-FW3-CDR3-FW4. The CDRs provide a specific antigen recognition site on the surface of the antibody.

Both Kabat and ImMunoGeneTics (IMGT) numbering nomenclature may be used herein. Generally, unless otherwise indicated (explicitly or by context) amino acid residues are numbered herein according to the Kabat numbering scheme (Kabat et al., 1991, J Immunol 147 (5): 1709-19). For those instances when the IMGT numbering scheme is used, amino acid residues are numbered herein according to the ImMunoGeneTics (IMGT) numbering scheme described in Lefranc et al., 2005, Dev Comp Immunol 29 (3): 185-203.

It is possible to take monoclonal and other antibodies and use techniques of recombinant DNA technology to produce other antibodies or chimeric molecules which generally retain the specificity of the original antibody. Such techniques may involve introducing the CDRs into a different immunoglobulin framework, or grafting variable regions onto a different immunoglobulin constant region. Introduction of the CDRs of one immunoglobulin into another immunoglobulin is described for example in EP-A-184187, GB2188638A or EP-A-239400. Alternatively, a hybridoma or other cell producing an antibody molecule may be subject to genetic mutation or other changes, which may or may not alter the binding specificity of antibodies produced.

Antibody humanisation involves the transfer, or “grafting”, of critical non-human amino acids onto a human antibody framework. Primarily this includes the grafting of amino acids in the complementarity-determining regions (CDRs), but potentially also other framework amino acids critical for the VH:VL interface and for orientation of the CDRs. Humanisation seeks to introduce human content to reduce the risk of immunogenicity, while retaining the original binding activity of the non-human parental antibody. The term “humanized antibody” is intended to refer to antibodies in which CDR sequences derived from the germline of another mammalian species have been grafted onto human framework sequences; optionally additional framework region modifications can be made within the human framework sequences. The term “humanised antibody” includes antibodies in which CDR sequences derived from the germline of another mammalian species have been grafted onto human framework sequences and optimized (for example by affinity maturation), e.g., by modification or one more amino acid residues in one or more of the CDRs and/or in one or more framework sequence to modulate or improve a biological property of the humanised antibody, e.g. to increase affinity, or to modulate the on rate and/or off rate for binding of the antibody to its target epitope. The term “humanised antibody” includes humanised antibody that has been optimized (for example by affinity maturation), thus humanised antibodies of the invention may be humanised, or both humanised and optimised, e.g. humanised and affinity matured.

As antibodies can be modified in a number of ways, the term “antigen-binding protein” or “antibody” should be construed as covering antibody fragments, derivatives, functional equivalents and homologues of antibodies, including any polypeptide comprising an immunoglobulin binding domain, an aptamer, affimer or bicyclic peptide, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023.

An example of an antibody fragment comprising both CDR sequences and CH3 domain is a minibody, which comprises a scFv joined to a CH3 domain (Hu et al. (1996) Cancer Res 56(13): 3055-61).

A domain (single-domain) antibody is a peptide, usually about 110 amino acids long, comprising one variable domain (VH) of a heavy-chain antibody, or of an IgG. A single-domain antibody (sdAb), (e.g., nanobody), is an antibody fragment consisting of a single monomeric variable antibody domain. Like a whole antibody (comprising two heavy and two light chains), it is an antigen-binding protein able to bind selectively to a specific antigen. Domain antibodies have a molecular weight of only 12-15 kDa and are thus much smaller than antibodies composed of two heavy protein chains and two light chains (150-160 kDa), and domain antibodies are even smaller than Fab fragments (˜50 kDa, one light chain and half a heavy chain) and single-chain variable fragments (˜25 kDa, two variable domains, one from a light and one from a heavy chain). Single-domain antibodies have been engineered from heavy-chain antibodies found in camelids; these are termed VHH fragments. Cartilaginous fish also have heavy-chain antibodies (IgNAR, ‘immunoglobulin new antigen receptor’), from which single-domain antibodies called VNAR fragments can be obtained. A domain (single-domain) antibody may be a VH or VL. A domain antibody may be a VH or VL of human or murine origin. Although most single-domain antibodies are heavy chain variable domains, light chain single-domain antibodies (VL) have also been shown to bind specifically to target epitopes.

Protein scaffolds have relatively defined three-dimensional structures and typically contain one or more regions which are amenable to specific or random amino acid sequence variation, to produce antigen-binding regions within the scaffold that are capable of binding to an antigen.

A humanised antibody or antigen-binding fragment of the invention binds to an epitope formed by residues 369-381 of human tau. Binding in this context may refer to specific binding. The term “specific” may refer to the situation in which the antibody molecule will not show any significant binding to molecules other than its specific binding partner(s), here an epitope within residues 369-381 of human tau. The term “specific” is also applicable where the antibody is specific for particular epitopes, such as an epitope comprised within residues 369-381 of human tau, that are carried by a number of antigens in which case the antibody molecule will be able to bind to the various antigens carrying the epitope. The epitope may be present in tau species that are monomeric, oligomeric or aggregates. Tau species may be full length or truncated in regions outside of residues 369-381. The epitope may be present in fragments of tau that comprise residues 369-381 (SEQ ID NO: 1) of human tau1-441 (SEQ ID NO: 2). Preferably humanised antibodies and antigen-binding fragments thereof of the invention bind to extracellular tau species characterised in that the epitope to which they bind is formed by residues of the amino acid sequence 373 to 379 (THKLTFR, SEQ ID NO: 150) of human 2N4R (amino acids 1-441) tau (SEQ ID NO: 2).

In particularly preferred aspects of the invention humanised antibodies and antigen-binding fragments thereof of the invention bind extracellular tau species characterised in that the epitope is formed and defined by residues of the amino acid sequence 373 to 379 (THKLTFR, SEQ ID NO: 150) of human 2N4R (amino acids 1-441) tau (SEQ ID NO: 2), wherein the epitope comprises residues K375, T377 and R379, preferably comprising residues T373, K375, T377 and R379 (the epitope bound by Clone 2, #44 and humanised versions thereof).

Binding in this context may refer to specific binding. The term “specific” may refer to the situation in which the antibody molecule will not show any significant binding to molecules other than its specific binding partner(s), here an epitope formed by residues of the amino acid sequence 373 to 379 (THKLTFR, SEQ ID NO: 150) of human 2N4R (amino acids 1-441) tau (SEQ ID NO: 2. The term “specific” is also applicable where the antibody molecule is specific for particular epitopes, such as an epitope formed by residues of the amino acid sequence 373 to 379 of human tau, as described herein, that are carried by a number of antigens in which case the antibody molecule will be able to bind to the various antigens carrying the epitope. The novel epitopes described herein may be present in tau species that are monomeric, oligomeric or aggregates. Tau species may be full length or truncated in regions outside of residues 373-379 The epitope may be present in fragments of tau that comprise residues 373-379 (SEQ ID NO: 150), of human tau1-441 (SEQ ID NO: 2).

Preferably humanised antibodies and antigen-binding fragments thereof of the invention bind to extracellular tau species characterised in that the epitope to which they bind is formed by residues of the amino acid sequence 373 to 379 (THKLTFR, SEQ ID NO: 150) of human 2N4R (amino acids 1-441) tau (SEQ ID NO: 2). In particularly preferred aspects of the invention humanised antibodies and antigen-binding fragments thereof bind extracellular tau species characterised in that the epitope is formed by residues of the amino acid sequence 373 to 379 (THKLTFR, SEQ ID NO: 150) of human 2N4R (amino acids 1-441) tau (SEQ ID NO: 2), wherein the epitope comprises residues: K375, T377 and R379, preferably comprising residues T373, K375, T377 and R379 (e.g., the epitope bound by Clone 2, #44).

Amino acids may be referred to by their one letter or three letter codes, or by their full name. The one and three letter codes, as well as the full names, of each of the twenty standard amino acids are set out below.

TABLE 2
Amino acids, one and three-letter codes.

| Amino acid | One letter code | Three letter code |
|---|---|---|
| alanine | A | Ala |
| arginine | R | Arg |
| asparagine | N | Asn |
| aspartic acid | D | Asp |
| cysteine | C | Cys |
| glutamic acid | E | Glu |
| glutamine | Q | Gln |
| glycine | G | Gly |
| histidine | H | His |
| isoleucine | I | lle |
| leucine | L | Leu |
| lysine | K | Lys |
| methionine | M | Met |
| phenylalanine | F | Phe |
| proline | P | Pro |
| serine | S | Ser |
| threonine | T | Thr |
| tryptophan | W | Trp |
| tyrosine | Y | Tyr |
| valine | V | Val |

In preferred embodiments, an antibody or an antigen-binding fragment thereof of the invention may comprise the set of six CDRs (HCDR1 (SEQ ID NO: 20), HCDR2 (SEQ ID NO: 21), HCDR3 (SEQ ID NO: 22), LCDR1 (SEQ ID NO: 23), LCDR2 (SEQ ID NO: 24), and LCDR3 (SEQ ID NO: 25)) of Clone 2 (#44) (e.g., as set forth in Table 5 when defined by Kabat nomenclature).

An antibody or an antigen-binding fragment thereof of the invention may comprise a humanised variant of the VH and VL sequence of clone 2 (SEQ ID NO: 118 and 119).

An antibody or an antigen-binding fragment thereof of the invention may comprise one or more, e.g., 1, 2, 3, 4, 5, 6, 7, 8, 9 or 10 further amino acid modifications in the VH and/or VL sequences, provided that functional properties of the antibody are retained.

A modification may be an amino acid substitution, deletion or insertion. Preferably, the modification is a substitution.

In preferred embodiments in which one or more amino acids are substituted with another amino acid, the substitutions may be conservative substitutions, for example according to the following table. In some embodiments, amino acids in the same category in the middle column are substituted for one another, i.e., a non-polar amino acid is substituted with another non-polar amino acid, for example. In some embodiments, amino acids in the same line in the rightmost column are substituted for one another.

TABLE 3
Amino acids

| | | |
|---|---|---|
| ALIPHATIC | Non-polar | G A P |
| | | I L V |
| | | C S T M |
| | Polar - uncharged | N Q |
| | Polar - charged | D E |
| | | K R |
| AROMATIC | | H F W Y |

In some embodiments, substitution(s) may be functionally conservative. That is, in some embodiments the substitution may not affect (or may not substantially affect) one or more functional properties (e.g., binding affinity) of the antibody molecule comprising the substitution as compared to the equivalent unsubstituted antibody molecule.

In a preferred embodiment, an antibody or an antigen-binding fragment thereof of the invention may comprise a VH and/or VL domain sequence with one or more amino acid sequence alterations (addition, deletion, substitution and/or insertion of an amino acid residue), preferably 20 alterations or fewer, 15 alterations or fewer, 10 alterations or fewer, 5 alterations or fewer, 4 alterations or fewer, 3 alterations or fewer, 2 alterations or fewer, or 1 alteration compared with the VH and/or VL sequences of the invention set forth herein.

In preferred embodiments, a humanised antibody or an antigen-binding fragment thereof of the invention may comprise a humanised VH domain sequence of clone 2 set forth in SEQ ID NO: 118, e.g., a humanised VH domain with an amino acid sequence which has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the sequence of Clone 2 set forth in SEQ ID NO: 118.

In preferred embodiments, a humanised antibody or an antigen-binding fragment thereof of the invention may comprise a humanised VH domain sequence of VH4VK4 of SEQ ID NO: 154, e.g., a humanised VH domain with an amino acid sequence which has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the VH sequence of Clone VH4VK4 of SEQ ID NO: 154.

In a preferred embodiment, a humanised antibody or an antigen-binding fragment thereof of the invention comprises a VH domain amino acid sequence comprising the set of HCDRs: HCDR1 (SEQ ID NO: 20), HCDR2 (SEQ ID NO: 21), and HCDR3 (SEQ ID NO: 22) of Clone 2, e.g., as set forth in Table 5 when defined by Kabat nomenclature and the VH domain has an amino acid sequence with at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the sequence of Clone 2 set forth in SEQ ID NO: 118.

In a preferred embodiment, a humanised antibody or an antigen-binding fragment thereof of the invention comprises a VH domain amino acid sequence comprising the set of HCDRs: HCDR1 (SEQ ID NO: 20), HCDR2 (SEQ ID NO: 21), and HCDR3 (SEQ ID NO: 22) of Clone VH4VK4 of SEQ ID NO: 154, when defined by Kabat nomenclature and the VH domain has an amino acid sequence with at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the sequence of Clone VH4VK4 of SEQ ID NO: 154.

In preferred embodiments, a humanised antibody or an antigen-binding fragment thereof of the invention may comprise a humanised VL domain amino acid sequence of Clone 2 set forth in SEQ ID NO: 119, e.g., a humanised VL domain with an amino acid sequence which has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the sequence of Clone 2 set forth in SEQ ID NO: 119.

In preferred embodiments, a humanised antibody or an antigen-binding fragment thereof of the invention may comprise a humanised VL domain amino acid sequence of Clone VH4VK4 of SEQ ID NO: 160, e.g., a humanised VL domain with an amino acid sequence which has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the sequence of VH4VK4 of SEQ ID NO: 160.

In a preferred embodiment, a humanised antibody or an antigen-binding fragment thereof of the invention comprises a VL domain comprising the set of LCDRs: LCDR1 (SEQ ID NO: 23), LCDR2 (SEQ ID NO: 24) and LCDR3 (SEQ ID NO: 25) of Clone 2 respectively, e.g., as set forth in Table 5 when defined by Kabat nomenclature and the VL domain has an amino acid sequence with at least 70%, at least 75%, at least 80%, %, at least 81%, at least 82%, at least 83%, at least 84%, at least 85%, %, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the sequence of Clone 2 set forth in SEQ ID NO: 119.

In a preferred embodiment, a humanised antibody or an antigen-binding fragment thereof of the invention comprises a VL domain comprising the set of LCDRs: LCDR1 (SEQ ID NO: 23), LCDR2 (SEQ ID NO: 24) and LCDR3 (SEQ ID NO: 25) of Clone VH4VK4 of SEQ ID NO: 160, respectively, when defined by Kabat nomenclature and the VL domain has an amino acid sequence with at least 70%, at least 75%, at least 80%, %, at least 81%, at least 82%, at least 83%, at least 84%, at least 85%, %, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the sequence of Clone VH4VK4 of SEQ ID NO: 160, respectively.

Sequence identity is commonly defined with reference to the algorithm GAP (Wisconsin GCG package, Accelerys Inc, San Diego USA). GAP uses the Needleman and Wunsch algorithm to align two complete sequences, maximising the number of matches and minimising the number of gaps. Generally, default parameters are used, with a gap creation penalty equaling 12 and a gap extension penalty equaling 4. Use of GAP may be preferred but other algorithms may be used, e.g. BLAST (which uses the method of Altschul et al. (1990) J. Mol. Biol. 215:405-410), FASTA (which uses the method of Pearson and Lipman (1988) PNAS USA 85:2444-2448), or the Smith-Waterman algorithm (Smith and Waterman (1981) J. Mol Biol. 147:195-197), or the TBLASTN program, of Altschul et al. (1990) supra, generally employing default parameters. In particular, the psi-Blast algorithm may be used (Nucl. Acids Res. (1997) 25 3389-3402). Sequence identity may be defined using the Bioedit, ClustalW algorithm.

Alignments were performed using Snapgene and based on MUSCLE (Multiple Sequence Comparison by Log-Expectation) algorithms (Edgar (2004a) Nucleic Acids Res 32:1792-7; Edgar (2004b) BMC Bioinformatics 5:113.).

The antibody may comprise a CH2 domain. The CH2 domain is preferably located at the N-terminus of the CH3 domain, as in the case in a human IgG molecule. The CH2 domain of the antibody is preferably the CH2 domain of human IgG1, IgG2, IgG3, or IgG4, more preferably the CH2 domain of human IgG1. The sequences of human IgG domains are known in the art.

The antibody may comprise an immunoglobulin hinge region, or part thereof, at the N-terminus of the CH2 domain. The immunoglobulin hinge region allows the two CH2-CH3 domain sequences to associate and form a dimer. Preferably, the hinge region, or part thereof, is a human IgG1, IgG2, IgG3 or IgG4 hinge region, or part thereof. More preferably, the hinge region, or part thereof, is an IgG1 hinge region, or part thereof.

The sequence of the CH3 domain is not particularly limited. Preferably, the CH3 domain is a human immunoglobulin G domain, such as a human IgG1, IgG2, IgG3, or IgG4 CH3 domain, most preferably a human IgG1 CH3 domain.

An antibody of the invention may comprise a human IgG1, IgG2, IgG3, or IgG4 constant region. The sequences of human IgG1, IgG2, IgG3, or IgG4 CH3 domains are known in the art. An antibody of the invention may comprise a human IgG constant region, e.g., a human IgG1 constant region.

An antibody of the invention may comprise a human IgG Fc with effector function.

Fc receptors (FcRs) are key immune regulatory receptors connecting the antibody mediated (humoral) immune response to cellular effector functions. Receptors for all classes of immunoglobulins have been identified, including FcγR (IgG), FcεRI (IgE), FcαRI (IgA), FcμR (IgM) and FcδR (IgD). There are three classes of receptors for human IgG found on leukocytes: CD64 (FcγRI), CD32 (FcγRIIa, FcγRIIb and FcγRIIc) and CD16 (FcγRIIIa and FcγRIIIb). FcγRI is classed as a high affinity receptor (nanomolar range KD) while FcγRII and FcγRIII are low to intermediate affinity (micromolar range KD).

In antibody dependent cellular cytotoxicity (ADCC), FcvRs on the surface of effector cells (natural killer cells, macrophages, monocytes and eosinophils) bind to the Fc region of an IgG which itself is bound to a target cell. Upon binding a signalling pathway is triggered which results in the secretion of various substances, such as lytic enzymes, perforin, granzymes and tumour necrosis factor, which mediate in the destruction of the target cell. The level of ADCC effector function various for IgG subtypes. Although this is dependent on the allotype and specific FcvR in simple terms ADCC effector function is high for human IgG1 and IgG3, and low for IgG2 and IgG4. See below for IgG subtype variation in effector functions, ranked in decreasing potency.

| Effector Function | Species | IgG Subtype Potency |
|---|---|---|
| ADCC | Human | IgG1 ≥ IgG3 >> IgG4 > IgG2 |
| | Mouse | IgG2b > IgG2a > IgG1 >> IgG3 |
| C1q Binding | Human | IgG3 > IgG1 >> IgG2 > IgG4 |
| | Mouse | IgG2a ≥ IgG2b > IgG3 > IgG1 |

FcγRs bind to IgG asymmetrically across the hinge and upper CH2 region. Knowledge of the binding site has resulted in engineering efforts to modulate IgG effector functions

Antibodies of the invention may have an Fc with effector function, enhanced effector function or with reduced effector function.

The potency of antibodies can be increased by enhancement of the ability to mediate cellular cytotoxicity functions, such as antibody-dependent cell-mediated cytotoxicity (ADCC) and antibody-dependent cell-mediated phagocytosis (ADCP). A number of mutations within the Fc domain have been identified that either directly or indirectly enhance binding of Fc receptors and significantly enhance cellular cytotoxicity: the mutations S239D/A330L/1332E (“3M”), F243L or G236A. Alternatively enhancement of effector function can be achieved by modifying the glycosylation of the Fc domain, FcγRs interact with the carbohydrates on the CH2 domain and the glycan composition has a substantial effect on effector function activity. Afucosylated (non-fucosylated) antibodies, exhibit greatly enhanced ADCC activity through increased binding to FcγRIIIa.

Activation of ADCC and CDC may be desirable for some therapeutic antibodies, however, in some embodiments, an antibody that does not activate effector functions is preferred.

Due to their lack of effector functions, IgG4 antibodies are the preferred IgG subclass for receptor blocking without cell depletion. However IgG4 molecules can exchange half-molecules in a dynamic process termed Fab-arm exchange. This phenomenon can occur between therapeutic antibodies and endogenous IgG4. The S228P mutation has been shown to prevent this recombination process allowing the design of IgG4 antibodies with a reduced propensity for Fab-arm exchange.

Fc engineering approaches have been used to determine the key interaction sites for the IgG1 Fc domain with Fcγ receptors and C1q and then mutate these positions to reduce or abolish binding. Through alanine scanning the binding site of C1q to a region covering the hinge and upper CH2 of the Fc domain was identified. The CH2 domain of an antibody or fragment of the invention may comprise one or more mutations to decrease or abrogate binding of the CH2 domain to one or more Fcγ receptors, such as FcγRI, FcγRIIa, FcγRIIb, FcγRIII and/or to complement. CH2 domains of human IgG domains normally bind to Fcγ receptors and complement, decreased binding to Fcγ receptors is expected to decrease antibody-dependent cell-mediated cytotoxicity (ADCC) and decreased binding to complement is expected to decrease the complement-dependent cytotoxicity (CDC) activity of the antibody molecule. Mutations to decrease or abrogate binding of the CH2 domain to one or more Fcγ receptors and/or complement are known in the art. An antibody molecule of the invention may comprise an Fc with modifications K322A/L234A/L235A or L234F/L235E/P331S (“TM”), which almost completely abolish FcγR and C1q binding. An antibody molecule of the invention may comprise a CH2 domain, wherein the CH2 domain comprises alanine residues at EU positions 234 and 235 (positions 1.3 and 1.2 by IMGT numbering) (“LALA mutation”). Furthermore, complement activation and ADCC can be decreased by mutation of Pro329 (position according to EU numbering), e.g., to either P329A or P329G. The antibody molecule of the invention may comprise a CH2 domain, wherein the CH2 domain comprises alanine residues at EU positions 234 and 235 (positions 1.3 and 1.2 by IMGT numbering) and an alanine (LALA-PA) or glycine (LALA-PG) at EU position 329 (position 114 by IMGT numbering). Additionally or alternatively an antibody molecule of the invention may comprise an alanine, glutamine or glycine at EU position 297 (position 84.4 by IMGT numbering).

Modification of glycosylation on asparagine 297 of the Fc domain, which is known to be required for optimal FcR interaction may confer a loss of binding to FcRs; a loss of binding to FcRs has been observed in N297 point mutations. An antibody molecule of the invention may comprise an Fc with an N297A, N297G or N297Q mutation. An antibody molecule of the invention with an aglycosyl Fc domain may be obtained by enzymatic deglycosylation, by recombinant expression in the presence of a glycosylation inhibitor, or following the expression of Fc domains in bacteria.

IgG naturally persists for a prolonged period in the serum due to FcRn-mediated recycling, giving it a typical half-life of approximately 21 days. Half-life can be extended by engineering the pH-dependent interaction of the Fc domain with FcRn to increase affinity at pH 6.0 while retaining minimal binding at pH 7.4. The T250Q/M428L variant, conferred an approximately 2-fold increase in IgG half-life (assessed in rhesus monkeys), while the M252Y/S254T/T256E variant (“YTE”), gave an approximately 4-fold increase in IgG half-life (assessed in cynomolgus monkeys). Extending half-life may allow the possibility of decreasing administration frequency, while maintaining or improving efficacy.

Immunoglobulins are known to have a modular architecture comprising discrete domains, which can be combined in a multitude of different ways to create multispecific, e.g. bispecific, trispecific, or tetraspecific antibody formats. Exemplary multispecific antibody formats are described in Spiess et al. (2015) Mol Immunol 67:95-106 and Kontermann (2012) Mabs 4 (2): 182-97, for example. The antibodies of the invention may be employed in such multispecific formats.

The invention provides a humanised or human antibody or antigen-binding fragment thereof, capable of competing with an antibody of the invention described herein (e.g., comprising a set of HCDR and LCDRs of Clone 2 (e.g., as listed in table 5 when defined by Kabat nomenclature) and/or a humanised variant of the VH and VL amino acid sequences of Clone 2), for binding to an isolated recombinant peptide comprising an epitope, said peptide comprising or consisting of residues 369-381 (SEQ ID NO: 1) of human 2N4R tau (SEQ ID NO: 2), when assessed in a competition assay. Preferably said epitope is formed and defined by residues of the amino acid sequence 373 to 379 (THKLTFR, SEQ ID NO: 150) of human 2N4R (amino acids 1-441) tau (SEQ ID NO: 2), preferably wherein the epitope comprises and is defined by residues: K375, T377 and R379, more preferably wherein the epitope comprises and is defined by residues T373, K375, T377 and R379 (e.g., the epitope bound by Clone 2, #44).

Competition assays include cell-based and cell-free binding assays including an immunoassay such as ELISA, HTRF, flow cytometry, fluorescent microvolume assay technology (FMAT) assay, Mirrorball, high content imaging based fluorescent immunoassays, radioligand binding assays, bio-layer interferometry (BLI), surface plasmon resonance (SPR) and thermal shift assays.

An antibody that binds to the same epitope as, or an epitope overlapping with, a reference antibody refers to an antibody that blocks binding of the reference antibody to its binding partner (e.g., an antigen or “target”) in a competition assay by 50% or more, and/or conversely, the reference antibody blocks binding of the antibody to its binding partner in a competition assay by 50% or more. Such antibodies are said to compete for binding to an epitope of interest.

An antigen-binding protein, such as an antibody or antigen-binding fragment thereof of the invention may be conjugated to a detectable label (for example, a radioisotope); or to a bioactive molecule. In this case, the antigen-binding protein, such as an antibody or antigen-binding fragment thereof may be referred to as a conjugate. Such conjugates may find application in the treatment and/or diagnosis of diseases as described herein. Such conjugates may find application for the detection (e.g., in vitro detection) an epitope comprising or consisting of residues 369-381 (SEQ ID NO: 1) of human 2N4R (amino acids 1-441) tau (SEQ ID NO: 2); preferably said epitope is formed by residues of the amino acid sequence 373 to 379 (THKLTFR, SEQ ID NO: 150) of human 2N4R (amino acids 1-441) tau (SEQ ID NO: 2), preferably wherein the epitope comprises residues: K375, T377 and R379, more preferably wherein the epitope comprising residues T373, K375, T377 and R379 (e.g., the epitope bound by Clone 2, #44).

The antigen-binding proteins of the invention (including conjugates) may be useful in the detection (e.g., in vitro detection) of an epitope of the invention (an epitope present on an isolated recombinant peptide consisting of residues 369-381 (SEQ ID NO: 1) of human 2N4R (amino acids 1-441) tau (SEQ ID NO: 2)); preferably said epitope is formed by residues of the amino acid sequence 373 to 379 (THKLTFR, SEQ ID NO: 150) of human 2N4R (amino acids 1-441) tau (SEQ ID NO: 2), preferably wherein the epitope is defined by residues: K375, T377 and R379, more preferably wherein the epitope is defined by residues T373, K375, T377 and R379 (e.g., the epitope bound by Clone 2, #44). Thus, the present invention relates to the use of an antigen-binding protein of the invention for detecting the presence of epitope of the invention in a sample. The antigen-binding protein may be conjugated to a detectable label as described elsewhere herein.

In a preferred embodiment, the present invention relates to an in vitro method of detecting an epitope of the invention in a sample, wherein the method comprises incubating an antigen-binding protein of the invention with a sample of interest, and determining binding of the antigen-binding protein to an epitope of the invention present in the sample, wherein binding of the antigen-binding protein indicates the presence of an epitope of the invention in the sample. Methods for detecting binding of an antigen-binding protein to its target antigen are known in the art and include ELISA, ICC, IHC, immunofluorescence, western blot, IP, SPR and flow cytometry.

The sample of interest may be a sample obtained from an individual. The individual may be human. Samples include, but are not limited to, tissue such as brain tissue, cerebro-spinal fluid (CSF), primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, saliva, sputum, tears, perspiration, mucus, tumour lysates, and tissue culture medium, tissue extracts such as homogenized tissue, tumour tissue, cellular extracts, and combinations thereof.

Following incubation, antigen-binding protein to antigen binding, e.g., antibody to antigen binding, is detected using an appropriate detection system. The method of detection can be direct or indirect, and may generate a fluorescent or chromogenic signal. Direct detection involves the use of primary antibodies that are directly conjugated to a label. Indirect detection methods employ a labelled secondary antibody raised against the primary antigen-binding protein, e.g., antibody, host species. Indirect methods may include amplification steps to increase signal intensity. Commonly used labels for the visualization (i.e., detection) of antigen-binding protein-antigen (e.g., antibody-epitope) interactions include fluorophores and enzymes that convert soluble substrates into insoluble, chromogenic end products.

The term “detecting” is used herein in the broadest sense to include both qualitative and quantitative measurements of a target molecule. Detecting includes identifying the mere presence of the target molecule in a sample as well as determining whether the target molecule is present in the sample at detectable levels. Detecting may be direct or indirect.

Suitable detectable labels which may be conjugated to antigen-binding proteins, such as antibodies, are known in the art and include radioisotopes such as iodine-125, iodine-131, yttrium-90, indium-111 and technetium-99; fluorochromes, such as fluorescein, rhodamine, phycoerythrin, Texas Red and cyanine dye derivatives for example, Cy7, Alexa750 and Alexa Fluor 647; chromogenic dyes, such as diaminobenzidine; latex beads; enzyme labels such as horseradish peroxidase; phospho or laser dyes with spectrally isolated absorption or emission characteristics; electro-chemiluminescent labels, such as SULFO-TAG which may be detected via stimulation with electricity in an appropriate chemical environment; and chemical moieties, such as biotin, which may be detected via binding to a specific cognate detectable moiety, e.g., labelled avidin or streptavidin.

An antigen-binding protein, such as an antibody or fragment thereof, of the invention may be conjugated to the detectable label by means of any suitable covalent or non-covalent linkage, such as a disulphide or peptide bond. Suitable peptide linkers are known in the art and may be 5 to 25, 5 to 20, 5 to 15, 10 to 25, 10 to 20, or 10 to 15 amino acids in length.

The invention also provides a nucleic acid or set of nucleic acids encoding an antibody or antigen-binding fragment of the invention, as well as a vector comprising such a nucleic acid or set of nucleic acids.

Where the nucleic acid encodes the VH and VL domain, or heavy and light chain, of an antibody molecule of the invention, the two domains or chains may be encoded on the same or on separate nucleic acid molecules.

An isolated nucleic acid molecule may be used to express an antibody molecule of the invention. The nucleic acid will generally be provided in the form of a recombinant vector for expression. Another aspect of the invention thus provides a vector comprising a nucleic acid as described above. Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the nucleic acid in a host cell. Vectors may be plasmids, viral e.g. phage, or phagemid, as appropriate.

A nucleic acid molecule or vector as described herein may be introduced into a host cell. Techniques for the introduction of nucleic acid or vectors into host cells are well established in the art and any suitable technique may be employed. A range of host cells suitable for the production of recombinant antibody molecules are known in the art, and include bacterial, yeast, insect or mammalian host cells. A preferred host cell is a mammalian cell, such as a CHO, NS0, or HEK cell, for example a HEK293 cell.

A recombinant host cell comprising a nucleic acid or the vector of the invention is also provided. Such a recombinant host cell may be used to produce an antigen-binding protein (e.g., antibody) of the invention. Thus, also provided is a method of producing an antigen-binding protein, e.g., antibody, of the invention, the method comprising culturing the recombinant host cell under conditions suitable for production of the antigen-binding protein, e.g., antibody. The method may further comprise a step of isolating and/or purifying the antigen-binding protein, e.g., antibody.

Thus the invention provides a method of producing an antigen-binding protein, e.g., antibody, of the invention comprising expressing a nucleic acid encoding the antigen-binding protein, e.g., antibody, in a host cell and optionally isolating and/or purifying the antigen-binding protein, e.g., antibody, thus produced. Methods for culturing host cells are well-known in the art. Techniques for the purification of recombinant antigen-binding proteins, e.g., antibodies, are well-known in the art and include, for example HPLC, FPLC or affinity chromatography, e.g., using Protein A or Protein L. In some embodiments, purification may be performed using an affinity tag on an antigen-binding protein, e.g., antibody. The method may also comprise formulating the antigen-binding protein, e.g., antibody, into a pharmaceutical composition, optionally with a pharmaceutically acceptable excipient or other substance as described below.

Antigen-binding proteins, e.g., antibodies, of the invention are expected to find application in therapeutic applications, in particular therapeutic applications in humans, for example in the treatment of a tauopathy, including but not limited to, a tauopathy selected from Alzheimer's disease (sporadic and monogenic familial forms), amyotrophic lateral sclerosis/parkinsonism-dementia complex, argyrophilic grains disease, beta-propeller protein associated neurodegeneration (BPAN), British type amyloid angiopathy, cerebral amyloid angiopathy, Creutzfeldt-Jakob disease, dementia pugilistica, diffuse neurofibrillary tangles with calcification, Down's syndrome, chronic traumatic encephalopathy (CTE), corticobasal degeneration (CBD), frontotemporal dementia (FTD), frontotemporal dementia and parkinsonism linked to chromosome 17 (FTDP-17), frontotemporal lobar degeneration, Gerstmann-Straussler-Scheinker disease, Hallervorden-Spatz disease, inclusion body myositis, multiple system atropy, myotonic dystrophy, Niemann-pick disease type C, non-guamanian motor neuron disease with neurofibrillary tangles, Parkinson's disease (sporadic and monogenic familial forms), Pick's disease, post-encephalitic parkinsonism, primary age-related tauopathy (PART), prion protein cerebral amyloid angiopathy, progressive subcortical gliosis, progressive supranuclear palsy (PSP), subacute sclerosing panencephalitis, tangle-dominant dementia, globular glial tauopathy, parkinsonism dementia complex of Guam, progressive non-fluent aphasia, multi-infarct dementia, ischemic stroke, traumatic brain injury (TBI) and stroke.

Also provided is a composition, such as a pharmaceutical composition, comprising an antigen-binding protein, e.g., antibody, according to the invention and an excipient, such as a pharmaceutically acceptable excipient.

The invention further provides an antigen-binding protein, e.g., antibody, of the invention, for use in a method of treatment. Also provided is a method of treating a patient, wherein the method comprises administering to the patient a therapeutically-effective amount of an antigen-binding protein, e.g., antibody, according to the invention. Further provided is the use of an antigen-binding protein, e.g., antibody, according to the invention for use in the manufacture of a medicament. A patient, as referred to herein, is preferably a human patient.

The invention also provides an antigen-binding protein, e.g., antibody, of the invention, for use in a method of treating a tauopathy, such as Alzheimer's disease, in a patient. Also provided is a method of treating a tauopathy, such as Alzheimer's disease, in a patient, wherein the method comprises administering to the patient a therapeutically-effective amount of an antigen-binding protein, e.g., antibody, according to the invention. Further provided is the use of an antigen-binding protein, e.g., antibody, according to the invention for use in the manufacture of a medicament for the treatment of a tauopathy, such as Alzheimer's disease, in a patient. The treatment may further comprise administering to the patient a second therapy, such as an FDA-approved AD medication, e.g., acetylcholinesterase inhibitors (e.g. donepezil), acetylcholine receptor positive modulators (e.g., Galantamine), NMDA receptor antagonists (e.g., memantine), or Parkinson's disease medications e.g., carbidopa-levodopa, dopamine receptor antagonists (e.g. pramipexole), monoamine oxidase B inhibitors (e.g., selegiline), catechol O-methyltransferase (COMT) inhibitors, amantadine, or anticholinergics (e.g., benztropine). The second therapy may be administered to the patient simultaneously, separately, or sequentially to the antigen-binding protein, e.g., antibody, of the invention.

In another aspect, the invention relates to an antigen-binding protein, e.g., antibody, of the invention for use in: a) treating a tauopathy, b) delaying progression of a tauopathy, c) preserving cognitive function of a patient suffering from a tauopathy, d) prolonging the survival of a patient suffering from a tauopathy e) reducing levels of free C-terminal tau in the CSF and/or serum, f) reducing levels of total tau in the CSF and/or serum, g) reducing the ratio of free C-terminal tau:total tau in the CSF and/or serum, h) reducing levels of neurofilament light chain protein (NfL) in CSF and/or serum, i) reducing total intracellular tau levels in neurons and/or astrocytes and/or microglia, j) reducing the rate of decline of whole brain volume and/or regional brain volume, k) reducing the rate of decline of functional connectivity of brain, l) improving functional connectivity of the brain, or m) reducing the brain tau burden based on PET or other imaging methodology.

The antigen-binding protein, e.g., antibody, as described herein may thus be for use for therapeutic applications, in particular for the treatment of a tauopathy, such as Alzheimer's disease.

An antigen-binding protein, e.g., antibody, as described herein may be used in a method of treatment of the human or animal body. Related aspects of the invention provide;
(i) an antigen-binding protein, e.g., antibody, described herein for use as a medicament,
(ii) an antigen-binding protein, e.g., antibody, described herein for use in a method of treatment of a disease or disorder,
(iii) the use of an antigen-binding protein, e.g., antibody, described herein in the manufacture of a medicament for use in the treatment of a disease or disorder; and,
(iv) a method of treating a disease or disorder in an individual, wherein the method comprises administering to the individual a therapeutically effective amount of an antigen-binding protein, e.g., antibody, as described herein.

The individual may be a patient, preferably a human patient.

Treatment may be any treatment or therapy in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, ameliorating, delaying, abating or arresting one or more symptoms and/or signs of the condition or prolonging survival of an individual or patient beyond that expected in the absence of treatment.

Treatment as a prophylactic measure (i.e., prophylaxis) is also included. For example, an individual susceptible to or at risk of the occurrence of a tauopathy, such as AD, may be treated as described herein. Such treatment may prevent or delay the occurrence of the disease in the individual.

A method of treatment as described may comprise administering at least one further treatment to the individual in addition to the antigen-binding protein, e.g., antibody. The antigen-binding protein, e.g., antibody, described herein may thus be administered to an individual alone or in combination with one or more other treatments. When the antigen-binding protein, e.g., antibody, is administered to the individual in combination with another treatment, the additional treatment may be administered to the individual concurrently with, sequentially to, or separately from the administration of the antigen-binding protein, e.g., antibody. Where the additional treatment is administered concurrently with the antigen-binding protein, e.g., antibody, the antigen-binding protein, e.g., antibody, and additional treatment may be administered to the individual as a combined preparation. For example, the additional therapy may be a known therapy or therapeutic agent for the disease to be treated.

Whilst an antigen-binding protein, e.g., antibody, may be administered alone, antigen-binding proteins, e.g., antibodies, will usually be administered in the form of a pharmaceutical composition, which may comprise at least one component in addition to the antigen-binding protein, e.g., antibody. Another aspect of the invention therefore provides a pharmaceutical composition comprising an antigen-binding protein, e.g., antibody, as described herein. A method comprising formulating an antigen-binding protein, e.g., antibody, into a pharmaceutical composition is also provided.

Pharmaceutical compositions may comprise, in addition to the antigen-binding protein, e.g., antibody, a pharmaceutically acceptable excipient, carrier, buffer, stabilizer or other materials well known to those skilled in the art. The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. The precise nature of the carrier or other material will depend on the route of administration, which may be by infusion, injection or any other suitable route, as discussed below.

For parenteral, for example subcutaneous or intravenous administration, e.g., by injection, the pharmaceutical composition comprising the antigen-binding protein, e.g., antibody, may be in the form of a parenterally acceptable aqueous solution which is pyrogen-free and has suitable pH, isotonicity and stability. Those of relevant skill in the art are able to prepare suitable solutions using, for example, isotonic vehicles, such as Sodium Chloride Injection, Ringer's Injection, or Lactated Ringer's Injection. Preservatives, stabilizers, buffers, antioxidants and/or other additives may be employed as required including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagines, histidine, arginine, or lysine; monosaccharides, disaccharides and other carbohydrates including glucose, mannose or dextrins; chelating agents, such as EDTA; sugars, such as sucrose, mannitol, trehalose or sorbitol; salt-forming counter-ions, such as sodium; metal complexes (e.g., Zn-protein complexes); and/or non-ionic surfactants, such as TWEEN™, PLURONICS™ or polyethylene glycol (PEG).

In some embodiments, antigen-binding proteins, e.g., antibodies may be provided in a lyophilised form for reconstitution prior to administration. For example, lyophilised antigen-binding proteins, e.g., antibodies may be reconstituted in sterile water or saline prior to administration to an individual.

Administration may be in a “therapeutically effective amount”, this being sufficient to show benefit to an individual. The actual amount administered, and rate and time-course of administration, will depend on the nature and severity of what is being treated, the particular individual being treated, the clinical condition of the individual, the cause of the disorder, the site of delivery of the composition, the type of antigen-binding protein, e.g., antibody, the method of administration, the scheduling of administration and other factors known to medical practitioners. Prescription of treatment, e.g., decisions on dosage etc., is within the responsibility of general practitioners and other medical doctors, and may depend on the severity of the symptoms and/or progression of a disease being treated. Appropriate doses of antigen-binding protein, e.g., antibodies, are well known in the art. A therapeutically effective amount or suitable dose of an antigen-binding protein, e.g., antibody, can be determined by comparing in vitro activity and in vivo activity in an animal model. Methods for extrapolation of effective dosages in mice and other test animals to humans are known. The precise dose will depend upon a number of factors, including whether the size and location of the area to be treated, and the precise nature of the antigen-binding protein, e.g., antibody.

A typical antibody dose is in the range 100 μg to 1 g for systemic applications, and 1 μg to 1 mg for topical applications. An initial higher loading dose, followed by one or more lower doses, may be administered. This is a dose for a single treatment of an adult individual, which may be proportionally adjusted for children and infants, and also adjusted for other antibody formats in proportion to molecular weight.

Treatments may be repeated at daily, twice-weekly, weekly or monthly intervals, at the discretion of the physician. The treatment schedule for an individual may be dependent on the pharmacokinetic and pharmacodynamic properties of the antibody composition, the route of administration and the nature of the condition being treated.

Treatment may be periodic, and the period between administrations may be about two weeks or more, e.g., about three weeks or more, about four weeks or more, about once a month or more, about five weeks or more, or about six weeks or more. For example, treatment may be every two to four weeks or every four to eight weeks. Suitable formulations and routes of administration are described above.

In a preferred embodiment, an antibody as described herein may be for use in a method of treating Alzheimer's disease.

In preferred embodiments, an antigen-binding protein, such as an antibody or an antigen-binding fragment thereof of the invention does not bind to an epitope comprised in residues 379-408 or 379-391 of human tau 2N4R.

### FIGURES

FIG. 1. Multiple species of tau released from human familial Alzheimer's disease neurons are not found in control neuron supernatants. Tau was immunoprecipitated (IP) from culture supernatants from non-disease control (NDC; lanes 1-3), familial Alzheimer's disease (fAD)-associated mutation, PSEN1 Y115C (PSEN; lanes 4-6) and frontotemporal dementia (FTD; lanes 7-9) associated mutation, MAPT IVS10+16 (MAPT) neurons, using commercial antibody, HT7 (lanes 3, 6, 9) or Tau-13 (lanes 2, 5, 8) and compared to an IgG control (1, 4, 7). Western blots show detection of tau species following each IP using anti-tau antibody (K9JA) (A). Bands highlighted and numbered 1-5 were excised and analysed by mass spectrometry.

FIG. 2. Anti-tau IgG clones #44 (clone 2) and #66 (clone 1) immunodeplete tau species from human iPSC derived neuronal secretomes. Conditioned media from NDC (pale grey) and 2 separate TS21 lines (dark grey, black) were immunodepleted with preimmune serum (1), K9JA polyclonal antibody (2), #44 (Clone 2) (3) and #66 (Clone 1) (4) and analysed by mid-region (MR tau; based on commercial antibodies BT2 and Tau5; A) and microtubule binding region (MTBR tau; based on commercially available antibody, K9JA; B) ELISAs. Preimmune serum served as a negative control to mock deplete samples. Values represent the mean+/−SD of three technical replicates.

FIG. 3. Anti-tau IgG clones #44 (Clone 2) and #66 (Clone 1) prevent tau-mediated blockade of long term potentiation (LTP) in vivo. Hippocampal LTP was measured in anesthetized rats in the presence of secretomes generated by trisomy 21 (TS21) iPSC-derived neuronal cultures. LTP was induced in vehicle treated animals (black bar; 1), but was inhibited in the presence of mock-depleted TS21 secretome (white bar; 2). LTP induction was observed in the presence of TS21 secretomes immunodepleted using IgG clone #44 (clone 2) (grey bar; 3) or #66 (clone 1) (checked bar; 4), indicating the removal of tau species responsible for the LTP block. The magnitude of LTP is expressed as the percentage of pre-stimulation baseline EPSP amplitude (±SEM) for n=4 (vehicle) or n=7 (secretome samples) animals. **, p<0.01; *, p<0.05; one-way ANOVA with Bonferroni multiple comparison test versus vehicle control (1); ####, p<0.0001, ##, p<0.01, paired t-test, post-versus pre-LTP induction.

FIG. 4. Human AD CSF contains C-terminal tau. CSF samples from 16 individuals with clinically confirmed Alzheimer's disease were pooled (final volume 8.5 mL) (A). 150 ng of clone #44 (clone 2) IgG was bound and cross-linked to protein A-coated beads, and the beads used to immunopurify tau present in the pooled AD CSF, which contained the target epitope (B). Proteins were digested on the beads with trypsin (C), and eluted peptides resolved by mass spectrometry (D). A C-terminal tau peptide was identified in the pooled AD CSF (E, shown as ‘X’), adjacent to the Gen2B epitope (shown as ‘Y’), confirming the presence of C-terminal tau fragments in AD CSF.

FIG. 5. Anti-tau antibodies bind to distinct epitopes within the immunogen sequence. Letter plot representations of epitope substitution scan analysis for antibody clones #44 (Clone 2; A) and #66 (Clone 1, B) highlight key residues required for binding to tau. Low level of binding of isotype control rabbit IgG to the peptide array (C) demonstrates that anti-tau antibody binding is CDR-specific. The linear peptides were generated bearing single amino acid substitutions at each position of the native lead peptide sequence, shown below the plot. Values obtained for replacements are indicated by the letter code for each replacement residue plotted at the height of the recorded value. Arrow indicates median value for the lead sequence.

FIG. 6. Humanised heavy chain (VH) and light chain (VK) sequences based on rabbit antibody clone #44 were designed using Composite Human Antibody Technology (Abzena). Alignments of the 6 VH chain (A) and 4 VK chain (B) sequences are shown, aligned to the original rabbit sequences (Parent). CDR definitions and protein sequence are numbered according to Kabat. Changes from the rabbit parental sequence are shaded.

FIG. 7. A panel of humanised anti-tau IgG bind to full length recombinant 2N4R tau in an ELISA format. Supernatants from HEK cells transiently transfected with each IgG clone were tested at 1:100 for binding to full length recombinant 2N4R tau (solid bars). No detectable binding to BSA was observed for any of the variants tested (empty bars). Binding of anti-tau hIgG1 was detected using an HRP-conjugated anti-human secondary antibody and data are shown from a representative experiment (n=1 replicate).

FIG. 8. Humanised anti-tau IgG bind to tau with high affinity. Representative SPR binding curves show clone #44 variants, VH0VK0 (A), VH3VK3 (B), VH3VK4 (C), VH4VK2 (D), VH4VK3 (E) and VH4VK4 (F) binding to full length recombinant 2N4R tau (0.39 nM to 12.5 nM applied at 2-fold dilutions). Experiments were run using a Biacore T200 with an association time of 60 s and a dissociation time of 200 s.

FIG. 9. Humanised antibody variants of clone #44 have Tm over 65° C. Fluorescence (triangles) and static light scattering at 473 nm (SLS; squares) signals from single replicates are shown, for the prioritised variants, VH3VK3 (A), VH3VK4 (B), VH4VK2 (C), VH4VK3 (D), VH4VK4 (E).

FIG. 10. Humanised monoclonal anti-tau antibodies inhibit monomeric tau uptake by neurons. Humanised variants of antibody clone #44, VH3VK3 (A), VH3VK4 (B), VH4VK2 (C), VH4VK3 (D), VH4VK4 (E) (solid triangles, solid line) or an isotype control human IgG1 (open squares, solid line) were incubated with full length pHrodo-labelled monomeric (P301S) 2N4R tau (25 nM) before imaging on the Incucyte S3. Mean orange (pHrodo) area per neuron (phase) area quantified every 60 mins for 21 h increased steadily over time in isotype and no antibody (solid circles, dashed line) treatments, but was significantly reduced in cells treated with anti-tau antibody variants. ***, p<0.001; one-way ANOVA with Tukey's multiple comparison test versus no antibody control. Negative control wells including no pHrodo-labelled tau are also shown (open circles, dashed line). Data are shown as mean+/−SEM of n=4 wells from one representative experiment

FIG. 11. Humanised monoclonal anti-tau antibodies inhibit aggregated tau uptake by human iPSC-derived neurons. Humanised variants of antibody clone #44, VH3VK3 (A), VH3VK4 (B), VH4VK2 (C), VH4VK3 (D), VH4VK4 (E) (solid triangles, solid line) or an isotype control human IgG1 (open squares, solid line) were incubated with full length pHrodo-labelled aggregated (P301S) 2N4R tau (50 nM) before imaging on the Incucyte S3. Mean orange (pHrodo) area per neuron (phase) area quantified every 60 mins for 21 h increased steadily over time in isotype and no antibody (solid circles, dashed line) treatments, but was significantly reduced in cells treated with anti-tau antibody variants. ***, p<0.001; one-way ANOVA with Tukey's multiple comparison test versus no antibody control. Negative control wells including no pHrodo-labelled tau are also shown (open circles, dashed line). Data are shown as mean+/−SEM of n=4 wells from one representative experiment.

FIG. 12. Humanised monoclonal anti-tau antibodies inhibit monomeric tau uptake by human iPSC-derived astrocytes. Humanised variants of antibody clone #44, VH3VK3 (A), VH3VK4 (B), VH4VK2 (C), VH4VK3 (D), VH4VK4 (E) (solid triangles, solid line) or an isotype control human IgG1 (open squares, solid line) were incubated with full length pHrodo-labelled monomeric (P301S) 2N4R tau (25 nM) before imaging on the Incucyte S3. Mean orange (pHrodo) area per astrocyte (phase) area quantified every 60 mins for 20 h increased steadily over time in isotype and no antibody (solid circles, dashed line) treatments, but was significantly reduced in cells treated with anti-tau antibody variants. ***, p<0.001; one-way ANOVA with Tukey's multiple comparison test versus no antibody control. Negative control wells including no pHrodo-labelled tau are also shown (open circles, dashed line). Data are shown as mean+/−SEM of n=4 wells from one representative experiment.

FIG. 13. Humanised monoclonal anti-tau antibodies inhibit aggregated tau uptake by human iPSC-derived astrocytes. Humanised variants of antibody clone #44, VH3VK3 (A), VH3VK4 (B), VH4VK2 (C), VH4VK3 (D), VH4VK4 (E) (solid triangles, solid line) or an isotype control human IgG1 (open squares, solid line) were incubated with full length pHrodo-labelled aggregated (P301S) 2N4R tau (50 nM) before imaging on the Incucyte S3. Mean orange (pHrodo) area per astrocyte (phase) area quantified every 60 mins for 20 h increased steadily over time in isotype and no antibody (solid circles, dashed line) treatments, but was significantly reduced in cells treated with anti-tau antibody variants. ***, p<0.001; one-way ANOVA with Tukey's multiple comparison test versus no antibody control. Negative control wells including no pHrodo-labelled tau are also shown (open circles, dashed line). Data are shown as mean+/−SEM of n=4 wells from one representative experiment.

FIG. 14. Humanised anti-tau human IgG1 increase uptake of monomeric tau by human iPSC-derived microglia. Humanised variants of antibody clone #44, VH3VK3 (A), VH3VK4 (B), VH4VK2 (C), VH4VK3 (D), VH4VK4 (E) (solid circles, solid line) or an isotype control hIgG1 (open squares, solid line) were incubated with full length pHrodo-labelled monomeric 2N4R before imaging on the Incucyte S3. Mean orange (pHrodo) area per microglial (phase) area quantified every 30 mins for 15 h increased moderately over time in isotype and no antibody (solid triangles, dashed line) treatments, but was significantly increased in cells treated with anti-tau antibody clones. Negative control wells including no pHrodo-labelled tau are also shown (open circles, dashed line). Data are given as mean+/−SEM of n=4 wells from one representative experiment. ***, p<0.001; **, p<0.005; one-way ANOVA with Tukey's multiple comparison test versus no antibody control.

FIG. 15. Humanised anti-tau human IgG1 increase uptake of aggregated tau by human iPSC-derived microglia. Humanised variants of antibody clone #44, VH3VK3 (A), VH3VK4 (B), VH4VK2 (C), VH4VK3 (D), VH4VK4 (E) (solid circles, solid line) or an isotype control hIgG1 (open squares, solid line) were incubated with full length pHrodo-labelled monomeric 2N4R before imaging on the Incucyte S3. Mean orange (pHrodo) area per microglial (phase) area quantified every 30 mins for 15 h increased moderately over time in isotype and no antibody (solid triangles, dashed line) treatments, but was significantly increased in cells treated with anti-tau antibody clones. Negative control wells including no pHrodo-labelled tau are also shown (open circles, dashed line). Data are given as mean+/−SEM of n=4 wells from one representative experiment. ***, p<0.001; **, p<0.005; one-way ANOVA with Tukey's multiple comparison test versus no antibody control.

FIG. 16. Purified humanised monoclonal anti-tau antibodies detect increased levels of tau in familial Alzheimer's disease (AD; Presenilin 1 mutation) compared to non-demented control (NDC) post-mortem cerebral cortex. Western blots of recombinant 2N4R tau (lane 1) compared to brain lysates from an NDC (lane 2) and an AD patient (lane 3) are shown. Clone #44 variants, VH3VK3 (A), VH3VK4 (B), VH4VK2 (C), VH4VK3 (D), VH4VK4 (E) and the parental VH0VK0 clone #44 (F) antibodies detect multiple species corresponding to different forms of tau, with increased detection of both high and low MW species in the AD sample. Arrows indicate disease-specific tau species not detected in NDC brain. Actin (**) and neuronal tubulin (*) are shown (G) and were included to control for loading and post-mortem protein degradation respectively.

FIG. 17. Anti-tau antibodies detect increased levels of tau in familial Alzheimer's disease (AD), sporadic AD and dementia with Lewy bodies (DLB) compared to non-demented control (NDC) post-mortem cerebral cortex. Western blots of cerebral cortex lysates from NDC (A, lanes 1-5; B-C, lanes 1-4); familial AD patients (A; lanes 6-10); sporadic AD patients (B, lanes 5-8) and DLB patients (C; lanes 5-8) are shown. Parental rabbit IgG clone #44 (i) and humanised variant #44 VH4VK4 (ii) behave similarly and detect multiple species corresponding to different forms of tau, with increased detection of both high and low MW species in Alzheimer's and DLB samples. Panels iii-vii show the same blots re-probed using commercially available anti-tau antibodies: Tau13 (iii.), HT7 (iv.), Tau5 (v.) and Tau46 (vi.), and highlight the clear disease specificity of tau species detected with antibodies targeting SEQ ID NO: 1, compared to N-terminal (Tau13), mid-region (HT7, Tau5) and far C-terminal (Tau46) antibodies. Arrows indicate tau species detected in disease samples, but not NDC, by antibodies targeting SEQ ID NO: 1 but not by commercially available antibodies. Actin (**) and neuronal tubulin (*) are shown (vii.) and were included to control for loading and post-mortem protein degradation respectively.

FIG. 18. Novel antibodies based on humanised antibody clone #44_VH4VK4 were generated by mutagenesis of VH CDR2 (H01-H06) and VH CDR3 (H06-H20) (Abzena) and VL CDR3 (L02). Alignments of the 20 top VH variants (#44_VH4VK4_H01 to _H20) and 4 recombined variants (#44_VH4VK4_H06-H01, _H06-H02, _H06-H04, H16-H04) (A) and 1 representative VK chain variant (#44_VH4VK4_L02) (B) sequences are shown, aligned to the parental humanised antibody clone #44_VH4VK4 sequence (P). CDR definitions and protein sequence are numbered according to Kabat. Changes from the parental humanised sequence are shaded.

FIG. 19. A panel of affinity matured #44_VH4VK4 hIgG1 variants bind to full length recombinant 2N4R tau in an ELISA format. Supernatants from HEK cells transiently transfected with each IgG clone were tested at 1:100 for binding to full length recombinant 2N4R tau (solid bars). No detectable binding to BSA was observed for any of the variants tested (empty bars). Binding of anti-tau hIgG1 was detected using an HRP-conjugated anti-human secondary antibody and data are shown from a representative experiment (n=1 replicate).

FIG. 20. Affinity matured humanised anti-tau IgG bind to tau with high affinity. Representative SPR binding curves show clone #44_VH4VK4 variants, _H01 (A) (VH SEQ ID No: 183, VL SEQ ID NO: 160), _H02 (B) (VH SEQ ID No: 184, VL SEQ ID NO: 160), _H04 (C) (VH SEQ ID No: 186, VL SEQ ID NO: 160), _H06 (D) (VH SEQ ID No: 188, VL SEQ ID NO: 160) and the parental #44_VH4VK4 (E) (VH SEQ ID No: 154, VL SEQ ID NO: 160) binding to full length recombinant 2N4R tau (0.39 nM to 50 nM applied at 2-fold dilutions). Experiments were run using a Biacore T200 with an association time of 60 s and a dissociation time of 200 s (cropped to 100s to improve fit). Antibodies were tested as hIgG1.

FIG. 21. Affinity matured humanised antibody variants of clone #44_VH4VK4, expressed as hIgG1 have Tm over 64° C. Fluorescence (triangles) and static light scattering at 473 nm (SLS; squares) signals from single replicates are shown, for the prioritised variants, _H01 (A), _H02 (B), _H04 (C), _H06 (D), compared to the parental #44_VH4VK4 (E).

FIG. 22. Affinity matured humanised monoclonal anti-tau antibodies inhibit tau uptake by human neurons. Affinity matured variants of humanised antibody clone #44_VH4VK4 (pVH/pVL), H01/pVL (A), H02/pVL (B), H04/pVL (C), H06/pVL (D), and the parental pVH/pVL (E) (solid triangles, solid line) or an isotype control human IgG1 (open squares, solid line) were incubated with full length pHrodo-labelled monomeric 2N4R tau 2N4R tau (25 nM) before imaging on the Incucyte S3. Mean orange (pHrodo) area per neuron (phase) area quantified every 60 mins for 18 h increased steadily over time in isotype and no antibody (solid circles, dashed line) treatments, but was significantly reduced in cells treated with anti-tau antibody variants. ***, p<0.001; one-way ANOVA with Dunnett's multiple comparison test versus no antibody control. Negative control wells including no pHrodo-labelled tau are also shown (open circles, dashed line). Data are shown as mean+/−SEM of n=4 wells from one representative experiment.

FIG. 23. Affinity matured humanised monoclonal anti-tau antibodies inhibit aggregated tau uptake by human neurons. Affinity matured humanised variants of antibody clone #44_VH4VK4 (pVH/pVL), H01/pVL (A), H02/pVL (B), H04/pVL (C), H06/pVL (D), and the parental pVH/pVL (E) (solid triangles, solid line) or an isotype control human IgG1 (open squares, solid line) were incubated with full length pHrodo-labelled aggregated (P301S) 2N4R tau (50 nM) before imaging on the Incucyte S3. Mean orange (pHrodo) area per neuron (phase) area quantified every 60 mins for 18 h increased steadily over time in isotype and no antibody (solid circles, dashed line) treatments, but was significantly reduced in cells treated with anti-tau antibody variants. ***, p<0.001; one-way ANOVA with Tukey's multiple comparison test versus no antibody control. Negative control wells including no pHrodo-labelled tau are also shown (open circles, dashed line). Data are shown as mean+/−SEM of n=4 wells from one representative experiment.

FIG. 24. Affinity matured humanised monoclonal anti-tau antibodies inhibit monomeric tau uptake by human astrocytes. Affinity matured humanised variants of antibody clone #44_VH4VK4 (pVH/pVL), H01/pVL (A), H02/pVL (B), H04/pVL (C), H06/pVL (D), and the parental pVH/pVL (E) (solid triangles, solid line) or an isotype control human IgG1 (open squares, solid line) were incubated with full length pHrodo-labelled monomeric (P301S) 2N4R tau (25 nM) before imaging on the Incucyte S3. Mean orange (pHrodo) area per astrocyte (phase) area quantified every 60 mins for 21 h increased steadily over time in isotype and no antibody (solid circles, dashed line) treatments, but was significantly reduced in cells treated with anti-tau antibody variants. ***, p<0.001; one-way ANOVA with Tukey's multiple comparison test versus no antibody control. Negative control wells including no pHrodo-labelled tau are also shown (open circles, dashed line). Data are shown as mean+/−SEM of n=4 wells from one representative experiment.

FIG. 25. Affinity matured humanised monoclonal anti-tau antibodies inhibit aggregated tau uptake by human astrocytes. Affinity matured humanised variants of antibody clone #44_VH4VK4 (pVH/pVL), H01/pVL (A), H02/pVL (B), H04/pVL (C), H06/pVL (D), and the parental pVH/pVL (E) (solid triangles, solid line) or an isotype control human IgG1 (open squares, solid line) were incubated with full length pHrodo-labelled aggregated (P301S) 2N4R tau (50 nM) before imaging on the Incucyte S3. Mean orange (pHrodo) area per astrocyte (phase) area quantified every 60 mins for 21 h increased steadily over time in isotype and no antibody (solid circles, dashed line) treatments, but was significantly reduced in cells treated with anti-tau antibody variants. ***, p<0.001; one-way ANOVA with Tukey's multiple comparison test versus no antibody control. Negative control wells including no pHrodo-labelled tau are also shown (open circles, dashed line). Data are shown as mean+/−SEM of n=4 wells from one representative experiment.

FIG. 26. Purified affinity matured humanised monoclonal anti-tau antibodies detect increased levels of tau in familial Alzheimer's disease (AD; Presenilin 1 mutation) compared to non-demented control (NDC) post-mortem cerebral cortex. Western blots of recombinant 2N4R tau (lane 1) compared to brain lysates from an NDC (lane 2) and an AD patient (lane 3) are shown. Clone #44 variants, pVH/pVL (parental) (A), H01/pVL (B), H02/pVL (C), H04/pVL (D), H06/pVL (E) and H16/pVL (F) antibodies detect multiple species corresponding to different forms of tau, with increased detection of both high and low MW species in the AD sample. Arrows indicate disease-specific tau species not detected in NDC brain. Equal quantities of protein were loaded onto each gel for each postmortem brain sample.

FIG. 27. Affinity matured, anti-tau antibodies detect increased levels of tau in a panel of familial Alzheimer's disease (AD), compared to non-demented control (NDC) post-mortem cerebral cortex. Western blots of cerebral cortex lysates from NDC (lanes 1-5); familial AD patients (lanes 6-10) are shown. Affinity matured humanised variant #44_VH4VK4_H01/pVL (A) H02/pVL (B), _H04/pVL (C) and _H06/pVL (D) detect multiple species corresponding to different forms of tau, with increased detection of both high and low MW species in familial Alzheimer's samples. Arrows indicate tau species detected in disease samples, but not NDC, by antibodies targeting SEQ ID NO: 1, Actin (**) and neuronal tubulin (*) are shown below and were included to control for loading and post-mortem protein degradation respectively.

FIG. 28. Affinity matured anti-tau antibodies detect increased levels of tau in sporadic AD and dementia with Lewy bodies (DLB) compared to non-demented control (NDC) post-mortem cerebral cortex. Western blots of cerebral cortex lysates from NDC (lanes 1-4); sporadic AD patients (A, C; lanes 5-8) and DLB patients (B, D; lanes 5-8) are shown. Affinity matured humanised variants #44_VH4VK4_H04/pVL (A, B) and #44_VH4VK4_H06/pVL (C, D) behave similarly and detect multiple species corresponding to different forms of tau, with increased detection of both high and low MW species in Alzheimer's and DLB samples. Arrows indicate tau species detected in disease samples, but not NDC, by antibodies targeting SEQ ID NO: 1, Actin (**) and neuronal tubulin (*) are shown below (E, F) and were included to control for loading and post-mortem protein degradation respectively.

FIG. 29. Humanised anti-tau antibodies bind to the same epitope within the immunogen sequence. Letter plot representations of epitope substitution scan analysis for antibody clones #44_VH4VK4_H04/pVL (Clone VH4VK4_H04; A) and #44_VH4VK4_H06/pVL (Clone VH4VK4_H06, B) highlight key residues required for binding to tau. Low level of binding of isotype control human IgG1 to the peptide array (C) is clearly distinct from binding of anti-tau antibodies and demonstrates that anti-tau antibody binding is CDR-specific. The linear peptides were generated bearing single amino acid substitutions at each position of the native lead peptide sequence, shown below the plot. Values obtained for replacements are indicated by the letter code for each replacement residue plotted at the height of the recorded value. Arrow indicates median value for the lead sequence.

### EXAMPLES

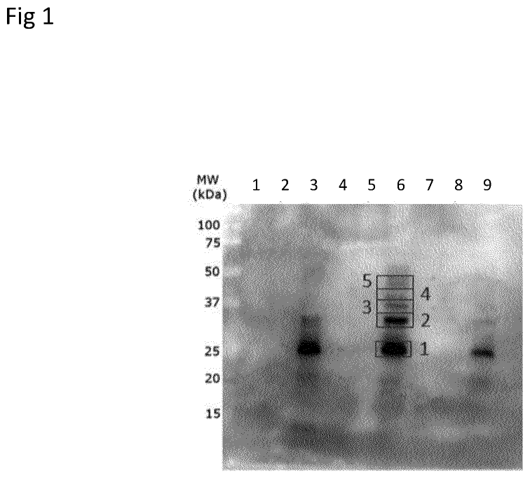

### Example 1: Multiple Species of Tau Released from Familial Alzheimer's Disease Neuronal Cultures are not Found in Control Culture Supernatants (FIG. 1)

Multiple species of tau released from familial Alzheimer's disease (fAD) and fronto-temporal dementia (FTD) neuronal cultures are not found in non-demented control (NDC) neuronal culture supernatants. Tau was immunoprecipitated (IP) from neuronal cell culture supernatants from NDC, fAD-associated mutation, PSEN1 Y115C (PSEN) and FTD-associated mutation, MAPT IVS10+16 (MAPT) using commercial antibody, HT7 (Invitrogen, Carlsbad, CA, USA) or Tau13 (Santa-Cruz, Dallas, TX, USA), and compared to a mouse monoclonal IgG control. Western blots (FIG. 1) show detection of tau species following each immunoprecipitation, using anti-tau antibody (K9JA; Agilent, Santa Clara, CA, USA). Bands (highlighted in boxes 1-5, FIG. 1) were excised and analysed by mass spectrometry to identify the tau peptides that are enriched in disease-related secretomes. This analysis reveals an increase in the number of peptides from the microtubule binding domain (corresponding to amino acids 260-267 (SEQ ID NO: 9), 306-317 (SEQ ID NO: 10) and 354-369 (SEQ ID NO: 11) of 2N4R tau) and C-terminus (corresponding to amino acids 396-406 (SEQ ID NO: 12) of 2N4R tau) in disease-associated secretomes compared to those from NDC (Table 4). Data show that tau species including the microtubule binding domain and neighbouring C-terminal regions are secreted at higher levels from disease-associated neurons compared to NDC and may therefore represent pathological or toxic forms of tau.

1.1 Cell culture of human iPSC-derived neurons: Differentiation of human pluripotent stem cells (iPSC) to projection neuron cultures was carried out as described by Shi et al., Nature Neurosci. 15 (3): 477-86 (2012). iPSC lines from different genetic backgrounds were used: NDC (Shi et al., Nature Neurosci. 15 (3): 477-86; Shi et al. Nature Protocols 7 (10): 1836-46 (2012)); trisomy 21 (TS21; Shi et al. Nature Protocols 7 (10): 1836-46 (2012)); PSEN1 Y115C mutation (PSEN; Moore et al. Cell Rep 11 (5): 689-96 (2015)); APP V7171 mutation (APP; Moore et al. Cell Rep 11 (5): 689-96 (2015); MAPT IVS10+16 (MAPT; Sposito et al. Hum Mol Genet 24 (18): 5260-5269 (2015)). Cells were plated out for individual experiments at day 40 in vitro and maintained to day 60+ (D60+), where days in vitro refers to days post-induction (as detailed later).

1.2 Immunoprecipitation and western blotting (FIG. 1): iPSC-derived neurons were cultured in 12 well plates (Corning, New York, USA) and matured to D60, after this, conditioned media was collected every 48 hours. Media was spun to remove cell debris and the supernatant stored at −20° C. Conditioned media was defrosted on ice and concentrated about 10-fold using Vivaspin 20, 10 kDa MWCO Polyethersulfone (Sigma, St Louise, MI, USA).

1.2.1 Antibody conjugation: Dynabeads (Thermo Fisher Scientific, Waltham, MA, USA) were washed prior to incubation with 5 μg specified antibody for 10 mins. IgG antibody bead mix were then added to concentrated conditioned media and incubated overnight on a roller. Dynabeads were removed from the conditioned media and replaced with Tau13 (Abcam, Cambridge, UK) antibody bead mix and incubated for ˜8 hours. Dynabeads were removed from the conditioned media and replaced with HT7 (Invitrogen, Carlsbad, CA, USA) antibody bead mix and incubated overnight. All beads were washed three times with 0.05% tween (PBS). 100 μl Laemlli lysis buffer were added to all beads and boiled for 10 mins. The supernatant was kept for running on SDS gel.

1.2.2 Western blotting: 20 μl of sample were loaded in 12% Mini-Protean TGX precast gel (Bio-Rad, Hercules, CA, USA) and transferred onto 0.2 μm PVDF membranes (GE Healthcare Life science, Chicago, IL, USA) at 200 mA for two hours at 4° C. Membranes were blocked in 5% dried skimmed milk (Marvel, Premier Foods, St Albans, UK), 0.1% Tween in PBS for 1 hour at room temperature (RT).

The protein-transferred membranes were probed overnight at RT with the primary antibody (at the concentration specified). Membranes were subsequently incubated with secondary antibody (goat anti-rabbit HRP) for 1 hour at RT.

1.3 Mass spectrometry: 20 μl of sample were loaded in 12% Mini-Protean TGX precast gel (Bio-Rad, Hercules, CA, USA). Gel were then incubated with EZBlue™ Gel Staining Reagent (Sigma, St Louis, MO, USA) for 4 hrs and then destained with ddH₂O overnight. Bands that corresponded to tau by western blot analysis were excised from the colloidal blue SDS-PAGE. Excised bands were subjected to 20° C., in 200 μl 100 mM ammonium bicarbonate/50% acetonitrile, followed by, reduction with 5 mM tris(2-carboxyethyl) phosphine. Then alkylation by addition of iodoacetamide (25 mM final concentration; each incubation for 30 min per step) then liquid was removed. Gel pieces were dried in vacuum for 10 min and 25 μl 100 mM ammonium bicarbonate containing 5 μg/mL modified trypsin (Promega, Madison, WI, USA) was added (digestion for 17 h at 37° C.). Peptides were recovered and desalted using μC18 ZipTip (Millipore, Burlington, MA, USA) and eluted to a maldi target plate using 1-2 μl alpha-cyano-4-hydroxycinnamic acid matrix (Sigma, St Louis, MO, USA) in 50% acetonitrile/0.1% trifluoroacetic acid. Peptide masses were determined using a Bruker ultraflextreme Maldi mass spectrometer in reflectron mode and ms/ms fragmentation performed in LIFT mode. Data analysis was with FlexAnalysis, BioTools and ProteinScape software (Bruker, Billerica, MA, USA). Database searches of the combined mass fingerprint-ms/ms data were performed using Mascot (http://www.matrixscience.com). (Table 4)

TABLE 4
Peptide fragments identified by mass spectrometry in samples prepared from excised
Bands 1-5 (FIG. 1) are summarised. The amino acid sequence and position within the full
length 2N4R tau sequence (SEQ ID NO: 2) are given, with the number of times each peptide
was identified.

| POSITION | SEQUENCE | BAND 1 | BAND 2 | BAND 3 | BAND 4 | BAND 5 |
|---|---|---|---|---|---|---|
| 6-23 | QEFEVMEDHAGTYGLGDR | 2 | 2 | 2 | 2 | 3 |
| | (SEQ ID NO: 3) | | | | | |
| 181-190 | TPPSSGEPPK | 0 | 5 | 0 | 4 | 0 |
| | (SEQ ID NO: 4) | | | | | |
| 195-209 | SGYSSPGSPGTPGSR | 4 | 4 | 2 | 4 | 4 |
| | (SEQ ID NO: 5) | | | | | |
| 210-224 | SRTPSLPTPPTREPK | 0 | 0 | 2 | 0 | 0 |
| | (SEQ ID NO: 6) | | | | | |
| 212-224 | TPSLPTPPTREPK | 4 | 4 | 6 | 4 | 4 |
| | (SEQ ID NO: 7) | | | | | |
| 243-254 | LQTAPVPMPDLK | 4 | 8 | 8 | 8 | 6 |
| | (SEQ ID NO: 8) | | | | | |
| 260-267 | IGSTENLK | 0 | 4 | 0 | 0 | 0 |
| | (SEQ ID NO: 9) | | | | | |
| 306-317 | VQIVYKPVDLSK | 2 | 4 | 4 | 3 | 2 |
| | (SEQ ID NO: 10) | | | | | |
| 354-369 | IGSLDNITHVPGGGNK | 0 | 2 | 0 | 0 | 0 |
| | (SEQ ID NO: 11) | | | | | |
| 396-406 | SPVVSGDTSPR | 0 | 4 | 4 | 4 | 4 |
| | (SEQ ID NO: 12) | | | | | |

### Example 2: Peptide Synthesis

To further investigate the importance of microtubule binding domain/C-terminal containing tau fragments in neurodegenerative disease, novel antibodies targeting this region were generated. Peptide sequence KKIETHKLTFREN (SEQ ID NO: 1), corresponding to amino acids 369-381 of 2N4R tau was selected as an immunogen to generate rabbit IgG, for a number of reasons. First, the sequence adjoins the microtubule binding region (MTBR) but, unlike the MTBR itself, shows low identity with other regions within the tau protein and with microtubule binding protein family members. This increases the probability that antibodies generated bind specifically and selectively to the target region in tau, with low risk of cross-reactivity with other regions/proteins. Second, the absence of putative phosphorylation sites simplifies the interpretation of data obtained, in an area where tau phosphorylation has been linked to pathological outcomes (Augustinack et al., Acta Neuropathol 103 (1): 26-35 (2002)). Antibodies targeting this peptide therefore enable the role of C-terminal containing tau species to be explored/targeted, without complication associated with binding to multiple sites and/or differential binding to phosphorylated/dephosphorylated tau. No similar antibodies are available in the public domain that meet these criteria.

Generation and characterisation of rabbit monoclonal IgG antibodies that bind specifically to an epitope formed by peptide sequence KKIETHKLTFREN (SEQ ID NO: 1), corresponding to amino acids 369-381 of 2N4R tau is described in International Patent Application No. PCT/EP2020/068314, filed on 29 Jun. 2020, published as WO/2020/260722 on 30 Dec. 2020, from which this application claims priority. The contents of International Patent Application No. PCT/EP2020/068314, published as WO/2020/260722, are incorporated herein in their entirety,

Antigenic peptide, [C]-KKIETHKLTFREN-amide (SEQ ID NO: 13) was synthesised by Cambridge Research Biochemicals (Billingham, UK) using standard techniques and shown to be >95% pure by HPLCPeptides for immunisation were conjugated to Keyhole Limpet Haemocyanin (KLH) through the free thiol on the N-terminal cysteine, via a maleimidobenzoyl-N-hydroxysuccinimide ester (MBS) linker. Peptides for use in the Single Plasma cell Interrogation (SPIN) protocol were conjugated to a biotinylated polymer using the free thiol on the N-terminal cysteine, using proprietary methods (Exonbio, San Diego, CA, USA).

### Example 3: Immunisation of Rabbits with Target Immunogen

3.1: Immunisation of rabbits with target immunogen: One New Zealand White rabbit was used to generate the rabbit monoclonal antibodies. The rabbit was immunised with 200 μg (prepared at a 1 mg/mL dilution) purified KLH-conjugated peptide ([C]-KKIETHKLTFREN (SEQ ID NO: 13), corresponding to amino acids 369-381 of 2N4R tau) at day 0 (in Freund's complete adjuvant), then every 19 days to day 76 (in Freund's complete adjuvant). Adjuvant and antigen boosts were given (i.p.) on day 94 and 97 respectively before final bleeds were taken on day 104 and antisera collected using standard methods (Hancock & O'Reilly Methods Mol Biol 295:27-40 (2005)).

Animal husbandry and the procedures used complied with the Animal Welfare Act, 1966 (US Animal and Plant Health Inspection Service).

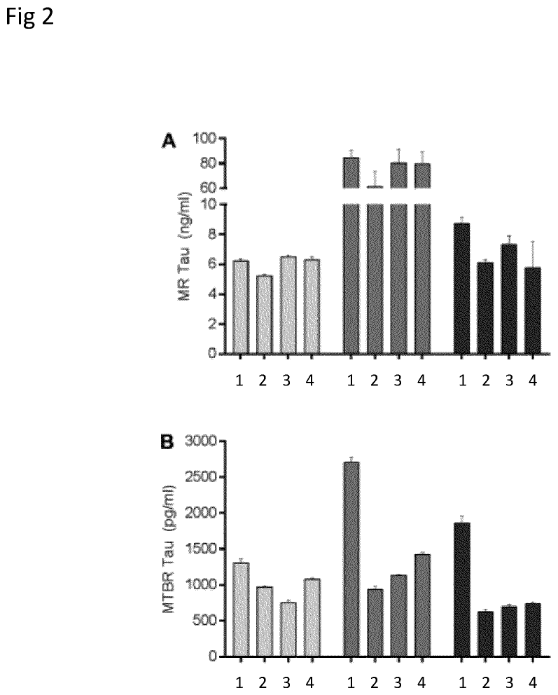

3.2: Peptide ELISA (FIG. 2): In order to confirm the generation of a robust immune response, serum was tested for immunoreactivity to the immobilised target antigen at various time points post-immunisation. Serum taken at day 76 post-immunisation showed immunoreactivity to the linear peptide target at dilutions of 1:1000-1:1000,000, indicative of IgG titres sufficient to proceed to monoclonal antibody isolation.

ELISA plates were coated with antigen (non-conjugated antigen peptide (Antigen peptide ([C]-KKIETHKLTFREN-amide (SEQ ID NO: 13); 2 μg/well in 1×PBS) overnight at 4° C. Antigen was removed from wells and the plates were blocked for 1 hour at RT with 5% dried milk in 1×PBS. Blocking solution was removed, 100 μL of diluted serum (diluted in 1% BSA/1×PBS) was added to relevant wells, and plates were incubated for 1 hour at RT with gentle shaking. Plates were then washed four times with PBS/0.1% Tween (PBST). Anti-rabbit IgG-HRP antibody (Sigma, St Louis, MO, USA), diluted 1:10,000 in 1% BSA in PBS, was added to each well and plates were incubated for 30 min at RT with gentle shaking before being washed four times with PBST. 50 μL 3,3′,5,5′-tetramethylbenzidine (TMB) ELISA solution was added to each well and plates were incubated for 15 mins at RT, an equal volume of 1 M sulfuric acid was added to each well and OD was measured at 450 nm.

### Example 4: Isolation of Monoclonal IgG Specific for the Target Epitope

96 individual antigen-specific plasma cells were identified and isolated using the target immunogen by Exonbio using proprietary methods (Exonbio, San Diego, CA, USA).

Splenocytes were isolated from the spleen of the immunised rabbit with Ficoll gradient (1.084) and were stained with plasma cell marker and biotin-conjugated antigen. Antigen-specific plasma cells were isolated and sorted into 96-well plates at one cell per well. Variable regions of antibody heavy and light chains were amplified individually by single cell polymerase chain reaction (PCR). Amplified heavy and light chains were then cloned into pRab293 plasmid and expressed in HEK293F suspension cells in serum-free medium using Invitrogen (Carlsbad, CA, USA) 293fectin transfection reagent, as per the manufacturer's instructions.

TABLE 5
Heavy and Light Chain amino acid CDR sequences for Clones -17

| | | | HC sequence | | | LC sequence | | |
|---|---|---|---|---|---|---|---|---|
| Clone | Code | Antibody | CDR1 | CDR2 | CDR3 | CDR1 | CDR2 | CDR3 |
| 1 | #66 | B9 | SNAMI | NIGTHGTTYYASWAKG | GDI | QASQSVDDNNNLA | EASTLAS | LGEFSCSSADCVV |
| | | | (SEQ ID NO: 14) | (SEQ ID NO: 15) | (SEQ ID NO: 16) | (SEQ ID NO: 17) | (SEQ ID NO: 18) | (SEQ ID NO: 19) |
| 2 | #44 | D6 | SYAMA | CIDRRGGTFYASWVKG | DSGAFDP | QASQSVYDNYLA | AASNLAS | LGEFSCTTTDCNV |
| | | | (SEQ ID NO: 20) | (SEQ ID NO: 21) | (SEQ ID NO: 22) | (SEQ ID NO: 23) | (SEQ ID NO: 24) | (SEQ ID NO: 25) |
| 3 | #12 | D2 | SYAVG | CIDSRDSKFYASWAKG | DSGAFNP | QASQSVYDNYLA | AVSNLAS | LGEFYCSSIDCNA |
| | | | (SEQ ID NO: 26) | (SEQ ID NO: 27) | (SEQ ID NO: 28) | (SEQ ID NO: 29) | (SEQ ID NO: 30) | (SEQ ID NO: 31) |
| 4 | #45 | E6 | SYAVG | CIDGRDSAFYASWAKG | DSGAFNP | QASQSVYDNYLS | AVSNLAS | LGEFYCSSIDCNA |
| | | | (SEQ ID NO: 32) | (SEQ ID NO: 33) | (SEQ ID NO: 34) | (SEQ ID NO: 35) | (SEQ ID NO: 36) | (SEQ ID NO: 37) |
| 5 | #61 | E8 | TYAMA | CIDRRGGTFYASWAKG | DSGAFDP | QASQSVYDNYLA | AASNLAS | LGEFSCTTTDCNV |
| | | | (SEQ ID NO: 38) | (SEQ ID NO: 39) | (SEQ ID NO: 40) | (SEQ ID NO: 41) | (SEQ ID NO: 42) | (SEQ ID NO: 43) |
| 6 | #34 | B5 | KNAMI | NIGTRGTTYYASWTKG | GDI | QSSQSVNNNDLA | EASTLAS | LGEFSCSSADCVA |
| | | | (SEQ ID NO: 44) | (SEQ ID NO: 45) | (SEQ ID NO: 46) | (SEQ ID NO: 47) | (SEQ ID NO: 48) | (SEQ ID NO: 49) |
| 7 | #43 | C6 | SYAMS | CIDSRGSVYYASWAKG | DSGAFDP | QASQSVYDNYLS | AASNLAS | LGEFYCSSMDCNA |
| | | | (SEQ ID NO: 50) | (SEQ ID NO: 51) | (SEQ ID NO: 52) | (SEQ ID NO: 53) | (SEQ ID NO: 54) | (SEQ ID NO: 55) |
| 8 | #41 | A6 | SNAMI | NIGTHGTTYYASWAKG | GDI | QASQSVDNNNNLA | EASTLAS | LGEFSCSSADCVA |
| | | | (SEQ ID NO: 56) | (SEQ ID NO: 57) | (SEQ ID NO: 58) | (SEQ ID NO: 59) | (SEQ ID NO: 60) | (SEQ ID NO: 61) |
| 9 | #87 | G11 | SYAVG | CIDSHDNTFYASWAKG | DSGAFNP | QASQSVYDNYLS | AVSNLAS | LGEFYCSSIDCNA |
| | | | (SEQ ID NO: 62) | (SEQ ID NO: 63) | (SEQ ID NO: 64) | (SEQ ID NO: 65) | (SEQ ID NO: 66) | (SEQ ID NO: 67) |
| 10 | #65 | A9 | SNAMI | NIGTHGTTYYASWSKG | GDI | QASQSVDNNNNLA | EASTLAS | LGEFSCSSADCVA |
| | | | (SEQ ID NO: 68) | (SEQ ID NO: 69) | (SEQ ID NO: 70) | (SEQ ID NO: 71) | (SEQ ID NO: 72) | (SEQ ID NO: 73) |
| 11 | #47 | G6 | NYAMA | CIDRRGGTFYASWAKG | DSGAFDP | QASQSVYDNYLA | AASNLAS | LGEFSCTTTDCNV |
| | | | (SEQ ID NO: 74) | (SEQ ID NO: 75) | (SEQ ID NO: 76) | (SEQ ID NO: 77) | (SEQ ID NO: 78) | (SEQ ID NO: 79) |
| 12 | #5 | E1 | SYAVG | CIDSHDNTFYASWAKS | DSGAFNP | QASQSVYDNYLS | AVSNLAS | LGEFYCSSIDCNA |
| | | | (SEQ ID NO: 80) | (SEQ ID NO: 81) | (SEQ ID NO: 82) | (SEQ ID NO: 83) | (SEQ ID NO: 84) | (SEQ ID NO: 85) |
| 13 | #37 | E5 | SYAMG | CIDRRGATFYASWAKG | DSGAFDP | QASQSVYDNYLS | AASNLAS | LGEFSCTTTDCNV |
| | | | (SEQ ID NO: 86) | (SEQ ID NO: 87) | (SEQ ID NO: 88) | (SEQ ID NO: 89) | (SEQ ID NO: 90) | (SEQ ID NO: 91) |
| 14 | #56 | H7 | SYAMG | CIDRRGGTFYASWAKG | DSGAFDP | QASQSVYDNYLA | AASNLAS | LGEFSCTTTDCNV |
| | | | (SEQ ID NO: 92) | (SEQ ID NO: 93) | (SEQ ID NO: 94) | (SEQ ID NO: 95) | (SEQ ID NO: 96) | (SEQ ID NO: 97) |
| 15 | #77 | E10 | SYAMT | CIDTGGSAYYASWAKG | DTGAFDP | QASQSVYDNNLA | AASNLPS | LGEFSCSSTDCNA |
| | | | (SEQ ID NO: 98) | (SEQ ID NO: 99) | (SEQ ID NO: 100) | (SEQ ID NO: 101) | (SEQ ID NO: 102) | (SEQ ID NO: 103) |
| 16 | #10 | B2 | SYAVG | CIDSRDSAFYASWAKG | DSGAFNP | QASQSVYDNYLS | AVSNLAS | LGEFYCSSIDCNA |
| | | | (SEQ ID NO: 104) | (SEQ ID NO: 105) | (SEQ ID NO: 106) | (SEQ ID NO: 107) | (SEQ ID NO: 108) | (SEQ ID NO: 109) |
| 17 | #69 | E9 | IYAMG | CIDRRGATFYATWAKG | DSGAFDP | QASQSVYDNNLA | AASNLAS | LGEFSCTTTDCNV |
| | | | (SEQ ID NO: 110) | (SEQ ID NO: 111) | (SEQ ID NO: 112) | (SEQ ID NO: 113) | (SEQ ID NO: 114) | (SEQ ID NO: 115) |

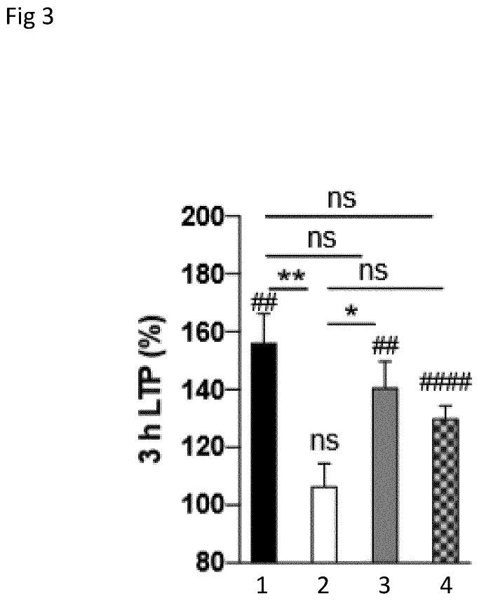

### Example 5: Transiently Expressed IgG Bind to the Isolated Peptide Immunogen (FIG. 3)

Individual rabbit IgG clones were transiently expressed in HEK293F cells in order to generate IgG samples for in vitro testing. Supernatants containing single IgG clones were tested for ability to bind to both the short peptide immunogen ([C]-KKIETHKLTFREN-amide; SEQ ID NO: 13) and full length 2N4R recombinant tau (SEQ ID NO: 2). 23 clones were found to bind full length tau with OD>0.3 and were prioritised for sequencing. Seventeen unique clones were identified and expressed (Table 5). Data demonstrate the utility of short peptide immunogen ([C]-KKIETHKLTFREN-amide) for the generation of IgG able to bind to full length recombinant 2N4R tau; and demonstrate the ability of the 17 prioritised clones to bind to both the immunogen and full length 2N4R tau.

### 5.1 Transient Expression of IgG in HEK Cells

Individual rabbit IgG clones were transiently expressed in HEK293F cells in order to generate IgG samples for in vitro testing. HEK293F cells cultured in suspension were transiently transfected with constructs in pRab293 plasmid using 293fectin transfection reagent (Invitrogen, Carlsbad, CA, USA) as per the manufacturer's instructions.

Supernatants were collected 7 days post-transfection. Antibodies were purified using a protein A column (25 mL resin) on an AKTA chromatography system (GE Healthcare, Chicago, IL, USA) and standard methods. Briefly, Protein A column was loaded with supernatant at 5 mL/min, then washed with PBS (5× total column volume). The protein peak was collected and dialysed in PBS overnight at 4° C.

For generation of mg quantities of IgG, 300 mL-1 litre HEK293F cells were transiently transfected and IgG was purified from culture media 7 days post-transfection using a protein A column, as above.

### 5.2 Peptide ELISA.

ELISA plates were coated with antigen (non-conjugated antigen peptide (Antigen peptide ([C]-KKIETHKLTFREN-amide (SEQ ID NO: 13)) or full length 2N4R tau (SEQ ID NO: 2), 100 ng/well; or 1% BSA in 1×TBS) in 1× carbonate-bicarbonate buffer for 1 hour at 37° C. Antigen was removed from wells and the plates were then blocked for 1 hour at RT with 5% dried milk in 1×TBS. Blocking solution was removed, HEK293F cell supernatant (10 μg/mL, to 0.0001 μg/mL in 1% BSA/1×TBS) was added to relevant wells, and plates were incubated for 1 hour at RT with gentle shaking. Plates were then washed four times with TBS/0.1% Tween (TBST). Anti-rabbit IgG-HRP antibody (Sigma, St Louis, MO, USA), diluted 1:5000 in 5% milk/TBS, was added to each well and plates were incubated for 1 hour at RT with gentle shaking before being washed four times with TBST. 3,3′,5,5′-tetramethylbenzidine (TMB) ELISA solution was added to each well and plates were incubated for 15 mins at RT. An equal volume of 1 M sulfuric acid was added to each well and OD was measured at 450 nm.

### Example 6: Monoclonal Antibodies Detect Full-Length Recombinant Tau by ELISA in a Concentration-Dependent Manner

Anti-tau rabbit IgG clones #66 (clone 1) and #44 (clone 2) bind to full length recombinant 2N4R tau (SEQ ID NO: 2), immobilised on an ELISA plate, in a concentration-dependent manner, with half maximal ELISA signal observed at 0.82 nM [0.7 to 0.95 nM] and 1.05 nM [0.96 to 1.15 nM] respectively (mean and 95% confidence intervals from n=2 wells in a single experiment are given). Data demonstrate high affinity binding of both clones to full length tau.

### Example 7: Monoclonal Anti-Tau Antibodies Detect Tau in Human iPSC-Derived Neuronal Cultures

Western blots demonstrated the ability of anti-tau IgG to detect recombinant and natively expressed tau. HEK293F cell-derived supernatants (generated as per Example 5.1) containing rabbit IgG clones #12 (Clone 3), #44 (Clone 2), #45 (clone 4) or #66 (Clone 1) detected full length recombinant 2N4R tau (SEQ ID NO: 2) (rPeptide, Watkinsville, GA, USA) as a dominant band at ˜60 kD. All clones tested were able to detect 22 ng tau loaded into a single lane. All 4 clones detected a dominant band at ˜50 kD in human iPSC-derived neuronal lysates. Treatment of neuronal lysates with lambda phosphatase to dephosphorylate proteins, decreased the apparent molecular weight of detected tau species detected in neuronal lysates (consistent with successful dephosphorylation), but did not impact the ability of antibodies to detect natively expressed tau. Data demonstrate that all 4 IgG clones tested bind equally well to phosphorylated and dephosphorylated tau samples and are able to detect both recombinant and natively expressed tau by western blot.

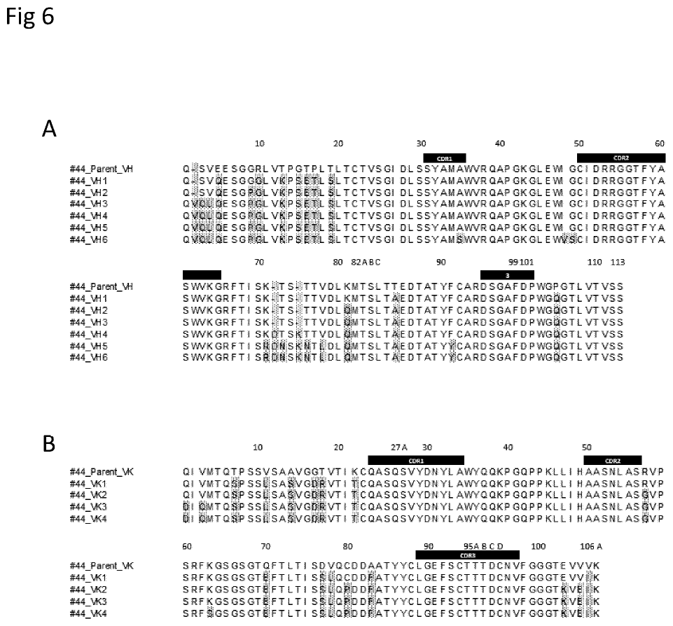

Purified clones #44 (Clone 2) and #66 (Clone 1) IgG detect tau in human iPSC-derived neurons from NDC, disease-AD-associated (PSEN Y115C, trisomy 21) and FTD-associated (MAPT IVS10+16) genetic backgrounds (FIG. 6). A dominant band is detected at ˜50 kD in each neuronal sample. Commercially available antibody, HT7 (raised against an epitope corresponding to amino acids 159-163 of 2N4R tau; Invitrogen, Carlsbad, CA, USA) also detected a dominant band at ˜50 kD when used to reprobe the blots, consistent with this representing full length tau. Data show that tau present intracellularly in human iPSC-derived neurons in culture can be detected by antibodies targeting the epitope corresponding to amino acids 369-381 (SEQ ID NO: 1) of 2N4R tau (SEQ ID NO: 2).

### Example 8: Monoclonal Anti-Tau Antibodies Detect Increased Levels of Tau in Familial Alzheimer's Disease (fAD) Compared to Non-Demented Control Post-Mortem Brain

Western blots were run using brain lysates from NDC and Alzheimer's disease patients. HEK293F cell-derived supernatants containing IgG clones #12 (Clone 3), #44 (Clone 2), #45 (Clone 4) or #66 (Clone 1) detect tau in human post-mortem brain samples. All 4 clones detect increased levels of tau in AD compared to NDC brain samples, including multiple high (>75 kD) and low (<40 kD) molecular weight species that are absent in the NDC samples tested. Data demonstrate that 4 distinct IgG clones targeting the sequence corresponding to amino acids 369-381 of 2N4R tau (SEQ ID NO: 1) all detect a similar pattern of disease-specific high and low molecular weight species of tau in AD brain lysates, in addition to bands at ˜50 kD that are present in NDC and disease samples. This suggests that observations made are generalisable to all antibodies binding to SEQ ID NO: 1.

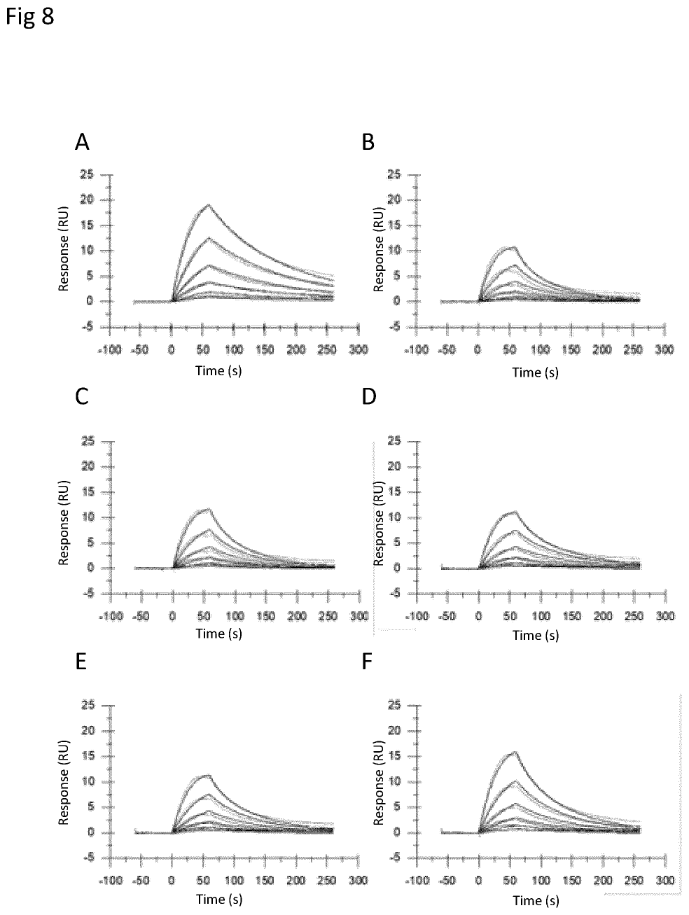

When a broader selection of post-mortem samples was assessed, purified clone #44 (Clone 2) and #66 (Clone 1) IgG again detected multiple species corresponding to different forms of tau, with increased detection of both high and low molecular weight species in Alzheimer's samples (FIG. 8). Actin and neuronal tubulin were included to control for loading and post-mortem protein degradation respectively, demonstrate that increased detection of tau in AD samples is not a result of increased protein loading or reduced degradation but rather reflects an increase in the abundance of C-terminal tau containing species in AD brain compared to NDC. Notably, antibodies targeting SEQ ID NO: 1 detected a distinct pattern of tau species in post-mortem brain samples that were not detected by commercially available anti-mid-region tau antibody, HT7. These include both low and high MW species that are clearly increased in abundance in AD compared to NDC brain samples. Low molecular weight species detected by the HT7 antibody are relatively unchanged or reduced in disease compared to non-disease brains. This suggests that disease-specific tau species are detected by the novel antibodies described here, that are not detected by commercially available mid-region antibodies. Data confirmed the presence and increased abundance of tau species containing the epitope of interest in Alzheimer's disease brain compared to controls.

8.1 Human brain samples: Human post-mortem brain samples were obtained from the Kings College London Neurodegenerative Diseases Brain Bank. All work was ethically approved and informed consent was obtained prior to brain donation. Alzheimer's disease brain samples were from the frontal cortex of individuals with familial Alzheimer's disease (PSEN1 mutations; summarised in Table 6). Non-demented control brain samples were from age-matched individuals who showed no clinical signs of dementia. Causes of death for the control individuals were: lung carcinoma (1), coronary artery occlusion (2), lung cancer (3), acute hepatic failure (4), metastatic prostate cancer (5); none of which would be predicted to impact tau levels/species detected post-mortem.

TABLE 6
A summary of the known mutations associated with familial
Alzheimer's disease present in the AD brain samples.

| Sample number | Disease-associated mutation |
|---|---|
| AD6 | PS1 (E280G) |
| AD7 | PS1 mutation |
| AD8 | PS1 mutation |
| AD9 | PS1 Delta4 truncation |
| AD10 | PS1 mutation |

### Example 9: Monoclonal Anti-Tau Antibodies Detect Increased Levels of Tau in Sporadic Alzheimer's Disease (AD), and Dementia with Lewy Bodies (DLB) Compared to Non-Demented Control Post-Mortem Brain

In order to extend the dataset beyond familial forms of AD, post-mortem brain samples from sporadic AD and DLB patients were assessed by western blot. Clone #66 (clone 1) IgG detected increased levels of both high and low molecular weight tau species in all disease associated samples when compared to NDC. As previously described (Example 8), commercially available mid-region tau antibodies, HT7 and BT2 (ThermoFisher, Waltham, MA), detected a range of predominantly lower molecular weight (<40 kD) tau species in all samples (in addition to full length tau at ˜50 kD), with no detectable disease-associated increases. Actin and neuronal tubulin controls confirm that changes in tau levels are not due to variations in protein and/or neuronal levels in samples shown. Data demonstrate the presence and increased abundance of tau species containing the epitope of interest (SEQ ID NO: 1) in sporadic AD and tauopathy brains, in addition to familial AD. This suggests that these species may be a general feature of AD and tauopathy and that therapeutics targeting this region may have broad utility in treating a range of tauopathies, in addition to both sporadic and familial forms of AD.

9.1 Human brain samples: See Example 8 for provenance of human post-mortem brain samples. All samples were from the frontal cortex of individuals with clinically and pathologically confirmed sporadic Alzheimer's disease (Braak stage 6) or DLB. Non-demented control brain samples were from age-matched individuals who showed no clinical signs of dementia or pathological signs of AD/tauopathy (Braak stage 0). Causes of death for the control individuals, where noted, would not be predicted to impact tau levels/species detected post-mortem.

### Example 10: Anti-Tau IgG Detect Natively Expressed Tau in Human iPSC-Derived Neuronal Cultures by Immunocytochemistry

Clone #66 (Clone 1) was used to visualise tau expression in NDC and FTD-associated (MAPT IVS10+16) iPSC-derived neurons (day 50+) by immunocytochemistry. Staining patterns are consistent with a predominantly axonal localisation of tau, with low levels of background. Data demonstrate that clone #66 (clone 1) is able to detect natively expressed tau, in situ in human neurons and is a useful tool for histological analyses.

### Example 11: Monoclonal Antibodies Detect Full Length Recombinant Tau in Sandwich Immunoassays

Anti-tau rabbit IgG clones #44 (Clone 2) and #66 (Clone 1) detect recombinant tau as part of an antibody pair in a MesoScale Discovery (MSD) assays. Standard curves were constructed using full length recombinant 2N4R tau (rPeptide, Watkinsville, GA, USA) and clone #44 (clone 2) or #66 (clone 1) as capture antibodies, in combination with either commercially available polyclonal antibody, K9JA (targeting amino acids 244-441; Agilent, Santa Clara, CA, USA), or commercially available monoclonal antibody, Tau5 (targeting amino acids 210-241; Thermo Fisher Scientific, Waltham, MA, USA). The limit of detection of each assay was approximately 80 pg/mL. Data demonstrate the utility of antibodies targeting the epitope of interest (SEQ ID NO: 1) in combination with commercially available monoclonal or polyclonal antibodies, for the detection of tau using sandwich immunoassays.

### Example 12: Anti-Tau IgGs Inhibit Uptake of Monomeric and Aggregated Tau into Human Neurons

Extracellular monomeric and aggregated tau is taken up by human neurons via a combination of endocytosis and macropinocytosis (Evans et al. (2018) Cell Rep 22 (13): 3612-3624). This process occurs physiologically, but is also proposed to play a role in the pathogenic spreading of toxic forms of tau observed in tauopathies, including Alzheimer's disease. Inhibiting uptake of toxic tau species is therefore predicted to be therapeutically beneficial in limiting the spread of tau pathology in the brain. Neuronal uptake of tau can be assessed and quantified by measuring fluorescence associated with tau labelled with the pH-sensitive dye, pHrodo. Increased fluorescence occurs following internalisation of labelled tau into the acidic endosome compartment, thereby providing a dynamic measure of tau uptake/internalisation. Anti-tau IgG clones, #44 (Clone 2) and #66 (Clone 1) inhibit the uptake of pHrodo-labelled monomeric tau by 65% and 71% inhibition respectively at 4 h and pHrodo-labelled aggregated tau by 56% and 47% inhibition respectively at 4 h into human iPSC-derived neurons. Isotype control antibody (monoclonal rabbit IgG; Thermo Fisher Scientific, Waltham, MA, USA) had no significant effect on tau uptake in this system.

Data demonstrate that antibodies targeting SEQ ID NO: 1 are able to reduce the uptake of tau species containing this epitope, by human neurons. Such antibodies would therefore be predicted to limit the neuron-to-neuron propagation of toxic tau species that include this epitope (SEQ ID NO: 1) in Alzheimer's disease and tauopathies and thereby reduce/slow the progression of clinical symptoms in patients.

12.1 Production of human iPSC-derived cerebral cortex neurons: As detailed in Example 1.1

12.2 Generation of aggregated (oligomeric) tau species: Tau P301S_10×his-tag_avi-tag was overexpressed in BL21 (DE3) bacteria. Cells were lysed using BugBuster (Millipore, Burlington, MA, USA) and clarified lysate was applied to a 5 mL HisTrapHP column (GE Healthcare, Chicago, IL, USA) in 2×PBS. Tau was eluted using a 0- to 500-mM imidazole gradient. Peak fractions were pooled and further purified in 2×PBS using a Superdex 200 16/60 gel filtration column (GE Healthcare, Chicago, IL, USA). Pooled fractions were then concentrated to approximately 8 mg/mL using a spin concentrator (Millipore, Burlington, MA, USA). Final protein concentration was determined by Nanodrop analysis.

1 mL tau P301S at 8 mg/mL was incubated with 4 mg/mL heparin (Sigma, St Louis, MO, USA) in PBS/30 mM 3-(N-morpholino) propanesulfonic acid (MOPS) (pH 7.2) at 37° C. for 72 h. Aggregated material was diluted in 9 mL PBS plus 1% (v/v) sarkosyl (Sigma, St Louis, MO, USA) and left rocking for 1 h at RT to completely solubilize any non-aggregated material. Insoluble tau was pelleted by ultracentrifugation for 1 h at 4° C. The pellet was resuspended in 1 mL PBS and sonicated at 100 W for 3×20 s (Hielscher UP200St ultrasonicator; Teltow, Germany) to disperse clumps or protein and break large filaments into smaller species.

12.3 Labelling of purified recombinant tau: Monomeric recombinant 2N4R tau was purchased from rPeptide (Watkinsville, GA, USA). Aggregated tau was prepared as described above. Recombinant monomeric tau (150 μM) or equivalent aggregated tau concentration (˜7 μg/mL) was incubated with 1.5 mM pHrodo Red Maleimide (dissolved in DMSO) and 1.5 mM tris(2-carboxyethyl) phosphine (1:10:10 molar ratio respectively) for 2 h in the dark at RT. Labelled samples were then subjected to size exclusion chromatography at 4° C. (Superdex 200 Increase 10/300 GL; GE Healthcare, Chicago, IL, USA) in 50 mM phosphate (pH 7.4) and 150 mM NaCl to remove unreacted dye. Oligomeric state of aggregates was assessed and found to be unaffected by labelling.

12.4 Quantification of tau uptake by human iPSC-derived cortical neurons: Monomeric tau (25 nM) and aggregated tau (50 nM) were prepared in N2B27 (Thermo Fisher Scientific, Waltham, MA, USA) and incubated with a 10-fold molar excess of antibody over tau (i.e. 250 and 50 nM IgG) for 90 min at 37° C. 100 μL antibody/tau mix was added to NDC neurons (day 60+) and fluorescence was imaged every 15 min for 4 h at 37° C./5% CO₂ from 18 fields per well using the Opera Phenix imaging system (Perkin Elmer, Waltham, MA, USA). Algorithms to identify ‘intense spots’ in the Alexa 568 channel were used to quantify the number of intense spots of fluorescence per well and these were plotted as mean+/−SEM from n=4 cells over time. One-way ANOVA with Dunnett's multiple comparison test was run vs no antibody control to determine significance.

### Example 13. Monoclonal Anti-Tau IgGs Immunodeplete Tau Species from Conditioned Media Obtained from Human iPSC-Derived Neurons (FIG. 2)

Secretomes were collected from human iPSC-derived neuronal cultures (generated as described in Example 1.1) at 48 hour intervals between days 70 and 80 post-neuronal induction. Secretomes were clarified by centrifugation before freezing at −20° C. Samples were thawed on ice and dialysed against artificial cerebrospinal fluid (aCSF). Immunodepletion of tau was achieved by 2 rounds of 12 hour incubations with monoclonal antibody and protein G agarose beads at 4° C. Preimmune serum from a rabbit was used as a control to mock deplete samples. Secretomes were collected from iPSC-derived neuronal cultures generated from two genetically distinct trisomy 21 lines, and from one NDC. Quantification of tau levels using a mid-region (BT2 (ThermoFisher, Waltham, MA)/Tau5 antibody pair) MSD assay and a microtubule binding region (MTBR; K9JA/K9JA antibody pair) assay confirmed the presence of elevated tau levels in trisomy 21 secretomes compared to NDC. In addition, MTBR tau levels were substantially (at least 4×) lower than mid-region tau, indicative of cleavage events leading to the generation of mid-region tau fragments that lack the MTBR and/or C-terminal domains, or the presence of tau species in which the MTBR and/or C-terminal epitopes are unavailable. Clones #44 (clone 2) and #66 (clone 1) deplete tau species from all three secretomes (FIG. 2). Clones #44 (Clone 2) and #66 (Clone 1) depleted MTBR containing tau to a greater extent in the trisomy 21 secretomes (line B, dark grey bars, 58% and 47% depletion by #44 (clone 2) and #66 (clone 1) respectively; and, line C, black bars, 62% and 60% depletion by #44 (clone 2) and #66 (clone 1) respectively) than in the NDC secretome (pale grey bars, 42% and 17% depletion by #44 (clone 2) and #66 (clone 1) respectively), indicating an increase in relative levels of tau species that include both the MTBR and the target epitope (SEQ ID NO: 1) in trisomy 21 compared to NDC secretomes. Clones #44 (clone 2) and #66 (clone 1) depleted mid-region tau only from TS21 secretomes (not NDC), most notably from one batch (line C; 16% and 34% respectively) indicating a disease-specific presence of tau species containing both mid-region and target epitopes (SEQ ID NO: 1). These data demonstrated a shift in the balance of different tau species (potentially due to disease-specific cleavage events, altered post-translational modification and/or conformational changes) present in disease compared to NDC, leading to an increase in both absolute tau levels and the proportion of MTBR and/or MR containing tau that can be detected by antibodies targeting SEQ ID NO: 1.

TABLE 8
Shows the immunodepletion efficiencey (in percent removed) of
antibodies tested, relative to pre-immune serum, based on tau
levels quantified using the mid-region and MTBR assays.

| | | | Clone #44 | Clone #66 |
|---|---|---|---|---|
| Line | Assay | K9JA | (clone 2) | (clone 1) |
| A (NDC) | Mid-region | 15.8 | N/A | N/A |
| | MTBR | 25.3 | 42.2 | 17.2 |
| B (TS21) | Mid-region | 27.4 | 5.0 | 6.1 |
| | MTBR | 65.1 | 57.9 | 47.2 |
| C (TS21) | Mid-region | 30.1 | 16.0 | 33.8 |
| | MTBR | 66.2 | 62.0 | 60.3 |

### Example 14. Tau-Mediated Blockade of In Vivo Long Term Potentiation (LTP) by Trisomy 21 Neuronal Secretomes is Prevented by Immunodepletion of Samples with IgG Clones #44 (Clone 2) or #66 (Clone 1) Prior to Dosing (FIG. 3)

Electrophysiology experiments were carried out on urethane-anesthetized (105-106 g/kg, intra-peritoneally) male Lister Hooded rats (250-350 g). Hippocampal LTP was measured by recording field excitatory postsynaptic potentials (EPSPs) from the stratum radiatum of CA1 in response to stimulation of the ipsilateral Schaffer collateral/commissural pathway before and after 200 Hz high frequency stimulation (HFS), as previously described (Hu et al. (2014) Nature Commun 5:3374). Secretomes were injected via cannula into the lateral ventricle of rats 30 min before the induction of synaptic plasticity.

All statistical analyses of LTP were conducted in v6.07 (GraphPad Software, La Jolla, CA, USA). The magnitude of LTP is expressed as the percentage of pre-HFS baseline EPSP amplitude (+SEM). The n refers to the number of animals per group. Control experiments were interleaved randomly throughout. For graphical representation, EPSP amplitudes were grouped into 5 min epochs; for statistical analyses, EPSP amplitudes were grouped into 10 min epochs. One way ANOVA with Sidak's multiple comparison test (one-way ANOVA-Sidak) was used for comparisons between groups of three or more. Two-way ANOVA with repeated measures with Sidak's multiple comparison test (two-way ANOVA RM-Sidak) was used when there were only two groups. Paired t tests were carried out to compare pre- and post-HFS values within groups. A value of p<0.05 was considered statistically significant.

The secretome isolated from trisomy 21 ‘line C’ (shown in FIG. 2) was tested. Mock-depleted samples significantly blocked the induction of LTP (p<0.01), compared to vehicle control treated animals, consistent with the presence of ‘toxic’ or inhibitory molecules in the secretome, as previously described (Hu et al., 2014, Nat Commun 5:3374). Trisomy 21 secretome that had been immune-depleted using either clone #44 (clone 2) or #66 (clone 1) to remove tau species including the target epitope (SEQ ID NO: 1) exhibited statistically significant LTP when compared to baseline (p<0.01 and p<0.001 respectively) (FIG. 3). Data demonstrate that the LTP block induced by Trisomy 21 secretomes can be prevented by removal of tau species that include the target epitope (SEQ ID NO: 1) and thereby show that C-terminal containing tau species are responsible for a component of the LTP block. Removal of these tau species in patients by administration of specific antibodies, would therefore be predicted to be therapeutically useful.

### Example 15: Monoclonal Anti-Tau Antibodies with Effector Function Increase Tau Uptake by Microglia

Microglia play an important role in clearing extracellular material in the central nervous system, to prevent accumulation of debris and enable repair processes to occur. In the context of neurodegenerative disease, phagocytosis of extracellular proteins, including aggregates, oligomers and monomeric forms, helps to reduce the extracellular concentrations of these species. Antibody clones #44 (Clone 2) and #66 (Clone 1) with effector function (i.e., rabbit IgG Fc) increase the uptake of both monomeric and aggregated tau by human iPSC-derived microglia compared to either tau alone or tau plus isotype control IgG conditions. Data suggest that therapeutic antibodies with effector function (e.g., formatted as hIgG1) would increase clearance of toxic forms of tau by microglia, and thereby reduce the extracellular concentration and deleterious effects of toxic forms of tau in the CNS. This activity would be predicted to be therapeutically beneficial.

### Example 16. Human AD CSF Contains C-Terminal Tau

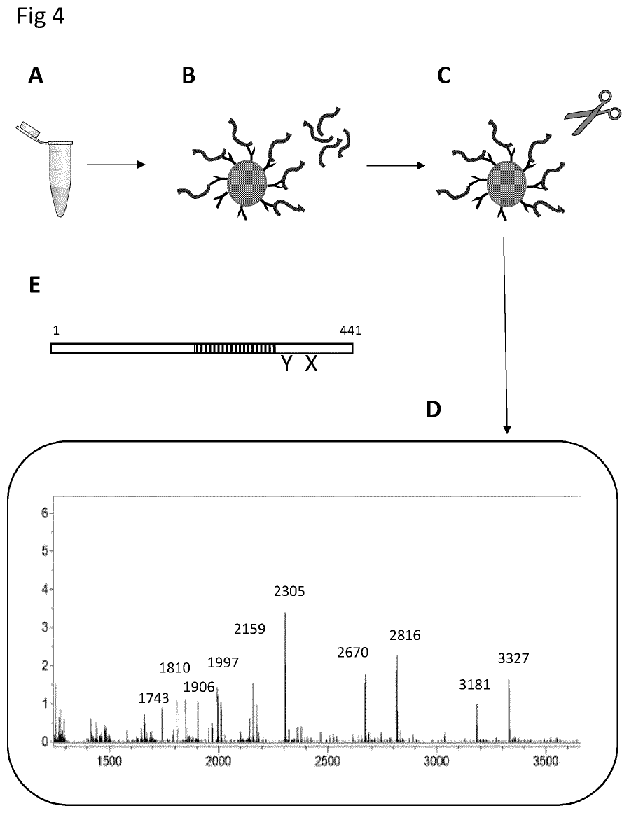

In order for an antibody to effectively target tau in vivo/in patients, relevant tau species must be present extracellularly. To demonstrate the presence of extracellular tau species containing the epitope of interest (SEQ ID NO: 1) we purified tau from pooled cerebrospinal fluid (CSF) samples obtained from AD patients using antibody clone #44 (clone 2). The bound proteins were then digested using trypsin and resolved by mass spectrometry (FIG. 4). Multiple tau peptides were identified, including a C-terminal peptide (SPVVSGDTSPR; corresponding to amino acids 396-406 of 2N4R tau; SEQ ID NO: 12) located adjacent to the antibody epitope. These data confirm the presence of tau species that include both the antibody epitope (SEQ ID NO: 1) and other C-terminal regions in AD CSF, and demonstrate the presence of such species extracellularly. These C-terminal tau-containing species are therefore targetable by therapeutic antibodies and present in fluids that could be exploited for biomarker detection.

### Example 17. Anti-Tau IgGs Inhibit Uptake of Monomeric and Aggregated Tau into Human Astrocytes

Limited information is available on the uptake of extracellular tau species by human astrocytes, although this is known to occur in rodents (Martini-Stoica et al. J Exp Med 215 (9): 2355-2377 (2018)). In addition, a recently described receptor for neuronal tau uptake, lipoprotein receptor-related protein 1 (LRP1), is reported to be expressed in astrocytes (Rauch et al. Nature 580(7803):381-385 (2020)), suggesting that the mechanisms of uptake may be shared. As a major cell type in the central nervous system, with putative roles in the propagation of tau pathology in Alzheimer's disease and tauopathy (reviewed in Sidoryk-Węgrzynowicz & Strużyńska Biochem J 476 (22): 3493-3504 (2019)) we explored whether antibodies targeting the sequence corresponding to amino acids 369 to 381 of 2N4R tau (SEQ ID NO: 1) have any impact on uptake of tau species by astrocytes.

Human iPSC-derived astrocytes readily take up both monomeric and aggregated tau species. Incubation of tau with anti-tau clone #44, significantly (P<0.001) inhibits uptake of monomeric 2N4R tau by 98.4±0.4% and of aggregated tau by 43.3±2.9% compared to uptake in the absence of antibody. Data provide evidence that therapeutic anti-tau antibodies targeting SEQ ID NO: 1, would reduce the uptake of toxic forms of tau by astrocytes, and thereby reduce the impact of astrocytes in the propagation of tau pathology. This activity is predicted to be therapeutically beneficial.

### Example 18. Monoclonal Anti-Tau Chimeric Human IgG1 Increase Uptake of Monomeric and Aggregated Tau by Human iPSC-Derived Microglia

As described in Example 15, monoclonal anti-tau rabbit IgG with effector function, increased uptake of both monomeric and aggregated tau by microglia. This increase in tau uptake is also observed with anti-tau human IgG1. Antibody clone #66 (Clone 1) expressed as a chimeric human IgG1 (i.e. with effector function) significantly increased the uptake of both monomeric (by 56±7%; P<0.001) and aggregated tau (by 59±9%; P<0.05) by human iPSC-derived microglia compared to uptake of tau alone. Isotype control human IgG1 had no significant effect on microglial uptake of monomeric (6.7±6% reduction) or aggregated tau (23±9% reduction), compared to baseline tau uptake in the absence of antibody. Data provided further evidence that therapeutic hIgG1 would increase clearance of extracellular tau by microglia, and thereby reduce the extracellular concentration and deleterious effects of extracellular forms of tau in the CNS. This activity is predicted to be beneficial therapeutically.

18.1 Production of chimeric hIgG1 antibodies: Chimeric hIgG1 were generated by Absolute Antibody (Oxford, UK) using the rabbit VH and VK sequences (SEQ ID NO: 116 and 117, Clone 1, #66) using proprietary methods (HEXpress™ service). Briefly, antibodies were produced following transient expression in HEK293 cells, affinity purified, buffer exchanged into phosphate buffered saline, sterile filtered and provided at a purity of >98% (based on SDS-PAGE) with <1 EU/mg endotoxin.

18.2 Production of human iPSC-derived microglia: Differentiation of human pluripotent stem cells (iPSC) to microglial cultures was carried out as described by Brownjohn et al. Stem Cell Rep 10 (4): 1294-1307 (2018). An iPSC line from an NDC background was used. Microglial progenitor cells were collected, plated in 96 well plates and maintained in complete microglia media (as described in Brownjohn et al., 2018) for approximately 14 days before use. On the day prior to use, cultures were switched into serum free media (RPMI 1640/Glutamax supplemented with 10 ng/ml GM-CSF and 100 ng/ml IL-34 (growth factors from Peprotech, NJ, US)) and phagocytosis experiments were completed in serum-free conditions.

18.3 Generation of aggregated (oligomeric) tau species: See Example 12.2

18.4 Labelling of purified recombinant tau: See Example 12.3.

P301S tau was used for both monomeric and aggregated tau preparations.

18.5 Quantification of tau uptake by human iPSC-derived microglia: Monomeric tau (25 nM) and aggregated tau (50 nM) were prepared in serum-free microglial media and incubated with a 1:10 ratio of antibody:tau (i.e. 2.5 and 5 nM IgG respectively) for 90 min at 37° C. Anti-tau hIgG1 was compared to an isotype control (anti-fluorescein [Apr. 4, 2020 (enhanced)], Absolute Antibody, Oxford, UK). 100 μL antibody/tau mix was added to iPSC-derived microglia and images were taken (bright field and orange channel) every 30 min for 16 h at 37° C./5% CO₂ from 9 fields per well using the Incucyte S3 imaging system (Sartorius, Göttingen, Germany). Algorithms to quantify (per well) the mean area of fluorescence in the orange channel (excitation: 513-568 nm), normalised to the mean area occupied by cells (phase area) and this was plotted as mean+/−SEM from n=4 cells over time. One-way ANOVA with Tukey's multiple comparison test was run vs no antibody control to determine significance.

### Example 19. Anti-Tau IgG Bind to Tau with High Affinity

Anti-tau IgGs bind to full length 2N4R tau (SEQ ID NO: 2) with high affinity. Clone #66 (Clone 1) and clone #44 (Clone 2) bind to full length recombinant 2N4R tau with Kos of 2.39 nM and 3.83 nM respectively (Table 9). It is predicted that the binding affinity of these antibodies would be equivalent to any tau species containing the epitope corresponding to amino acids 369-381 of 2N4R tau (SEQ ID NO: 1), if this sequence is accessible.

19.1 Production of chimeric hIgG1 antibodies: Clone #66 hIgG1 was generated as described in Example 18.1. Clone #44 hIgG1 was generated by Abzena (Cambridge, UK) using the rabbit VH and VK sequences (SEQ ID NO: 118 and 119, Clone 2, #44 as parental VH and VK) and using proprietary methods. Briefly, antibodies were produced following transient expression in CHO cells, affinity purified, buffer exchanged into phosphate buffered saline, sterile filtered and provided at a purity of >98% (following size exclusion chromatography).

19.2 Assessment of antibody binding to tau: Binding of anti-tau hIgG1 to full length recombinant 2N4R tau (Rpeptide; SEQ ID NO: 2) was assessed using the Biacore T200 (GE Healthcare, Chicago, IL, USA) running Biacore T200 Evaluation Software V2.0.1. hIgG1 were immobilised on a Protein A capture sensor chip in running buffer (HBS-EP+ buffer containing 1 mg/mL BSA) at 25° C., captured to ˜50 RU at 10 μL/min. For multi-cycle kinetics experiments (clone #66), recombinant 2N4R tau was flowed at concentrations ranging from 0.39 nM to 50 nM in running buffer at 40 μL/min, with an association time of 150 s and a dissociation time of 250 s. Optimised conditions for multiple-cycle kinetics experiments were applied to clone #44: recombinant 2N4R tau was flowed at concentrations ranging from 0.39 nM to 12.5 nM (2-fold dilutions) with an association time of 60 s and a dissociation time of 200 s (cropped to 65 s to improve analysis fit). Curves were compared to a reference cell that was mock immobilized (no antibody present).

Data were analysed using Langmuir (1:1) binding analysis, describing a 1:1 interaction at the surface:

$$A + B {{\vec{←}}_{{k}_{d}}}^{{k}_{a}} AB$$

$${K}_{D} = \frac{{k}_{d}}{{k}_{a}}$$

Where: kₐ is the association rate constant (M⁻¹s⁻¹) and k_{d} is the dissociation rate constant (s⁻¹)

Closeness of fit was judged in terms of the Chi square value, which describes the deviation between the experimental and fitted curves:

$$Chi square = \frac{\sum {( {r}_{f} - {r}_{x} )}^{2}}{n - p}$$

Where: rf is the fitted value at a given point, rx is the experimental value at the same point, n is the number of data points, p is the number of fitted parameters. The fitting algorithm sought to minimise Chi square.

TABLE 9
Summary of MCK binding analysis data for clones #66 (Clone 1) and
#44 (Clone 2) binding to full length recombinant 2N4R tau.

| Antibody | Kₐ (1/Ms) | K_{d} (1/s) | K_{D} (M) | R_{MAX} | Chi² (RU²) |
|---|---|---|---|---|---|
| #66 | 2.01E+06 | 0.004783 | 2.39E−09 | 25.98 | 0.194 |
| #44 | 2.07E+06 | 9.68E−03 | 4.68E−09 | 30.5 | 0.041 |

### Example 20: Anti-Tau Antibodies Bind to Distinct Epitopes within the Region ₃₇₃THKLTFR₃₇₉

Epitope fine mapping was carried out to identify critical residues within the amino acids 369-381 of 2N4R tau; SEQ ID NO: 1 that are required for antibody binding. In a replacement analysis, each residue is mutated to other amino acids to evaluate the importance of the residue for binding to the antibody.

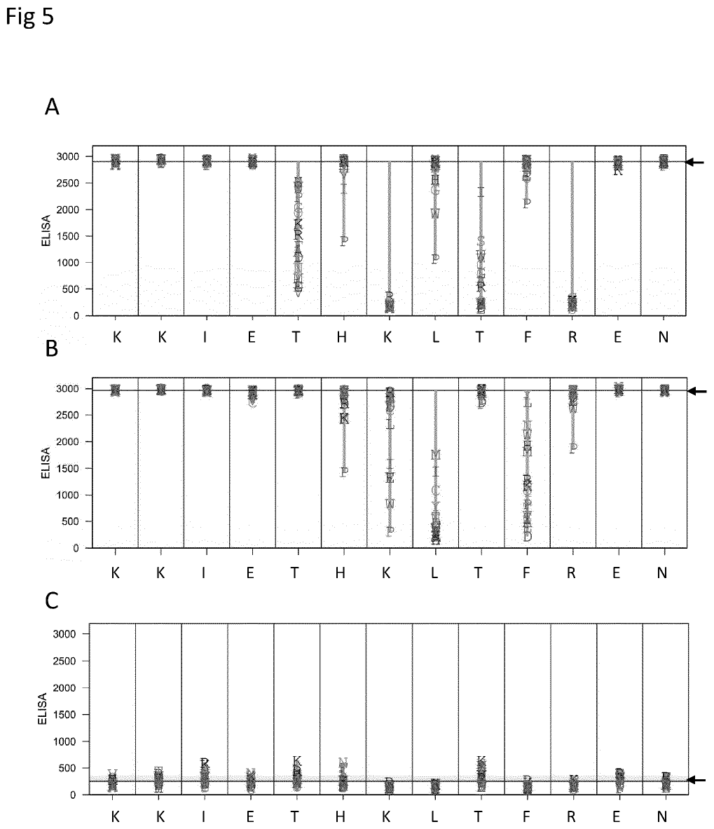

For antibody clone #44 (Clone 2), the replacement analysis shows that amino acid residues in the region, ₃₇₃THKLTFR₃₇₉ are important for binding (FIG. 5A). Specifically, residues, T₃₇₃, K₃₇₅, T₃₇₇ and R₃₇₉, as substitution of these residues results in a drop of intensity for all substitutes. Substitution of K₃₇₅, T₃₇₇ and R₃₇₉, drops signal intensities to background level, suggesting that they are critical for binding of this clone.

For antibody clone #66 (Clone 1), ₃₇₄HKL₃₇₆ and ₃₇₈FR₃₇₉ are important for binding (FIG. 5B). Specifically, substitution of L₃₇₆ or F₃₇₈ results in reduced antibody binding for all substitutes, suggesting a key role of these residues.

Little binding of the isotype control rabbit IgG is detected in this system (FIG. 5C), indicating that the ELISA signal obtained for anti-tau antibody clones #44 and #66 is CDR-specific.

Data demonstrate that the antibodies described here, exemplified by clones #44 (Clone 2) and #66 (Clone 1) bind to different specific epitopes within the peptide sequence (amino acids 369- 381 of 2N4R tau; SEQ ID NO: 1). The antibodies described share functional properties, indicating that it is the epitopes within and formed by this sequence, that dictate functional outcome.

20.1 Epitope substitution scan analysis—peptide synthesis: Replacement analysis was conducted by Pepscan Presto BV (Lelystad, The Netherlands) using proprietary methods. Briefly, a library of peptides was synthesised using Fmoc-based solid-phase peptide synthesis. An amino functionalized polypropylene support was obtained by grafting with a proprietary hydrophilic polymer formulation, followed by reaction with t-butyloxycarbonyl-hexamethylenediamine (BocHMDA) using dicyclohexylcarbodiimide (DCC) with N-hydroxybenzotriazole (HOBt) and subsequent cleavage of the Boc-groups using trifluoroacetic acid (TFA). Standard Fmoc-peptide synthesis was used to synthesize peptides on the amino-functionalized solid support by custom modified JANUS liquid handling stations (Perkin Elmer).

Peptides were designed based on the starting peptide (₃₆₉KKIETHKLTFREN₃₈₁; SEQ ID NO: 1) such that each amino acid was mutated one at a time, to every other natural amino acid. The order of peptides on the mini-cards was randomised and data were compared to that obtained with an isotype control antibody (rabbit IgG; Abcam, Cambridge, UK).

20.2 Epitope substitution scan analysis-ELISA screening: The binding of antibody to each of the synthesized peptides was tested in a Pepscan-based ELISA. The peptide arrays were incubated with primary antibody solution (5 μg/mL; overnight at 4° C.). After washing, the peptide arrays were incubated with a 1/1000 dilution of a swine anti-rabbit IgG peroxidase conjugate (DAKO, Jena, Germany) for 1 h at 25° C. After washing, the peroxidase substrate 2,2′-azino-di-3-ethylbenzthiazoline sulfonate (ABTS) and 20 L/mL of 3% H₂O₂ were added. After 1 h, the colour development was measured. The colour development was quantified with a charge coupled device (CCD)-camera and an image processing system. Values obtained from the CCD camera are quoted (range: 0 to 3000 mAU).

Data are presented as letter plots showing ELISA signal obtained for each peptide tested. Observed deviations from the maximum ELISA signal are indicative of mutations associated with altered (reduced) binding of the tested antibody to the target peptide.

### Example 21: Humanisation of Antibody Clone #44

Rabbit antibody clone #44 was humanised using Composite Human Antibody Technology™, developed by Antitope and commercialised by Abzena. The aim of the humanisation process is to reduce the potential for immunogenicity associated with using a non-human monoclonal antibody as a chronic therapeutic treatment, while retaining antigen binding affinity of the parental antibody.

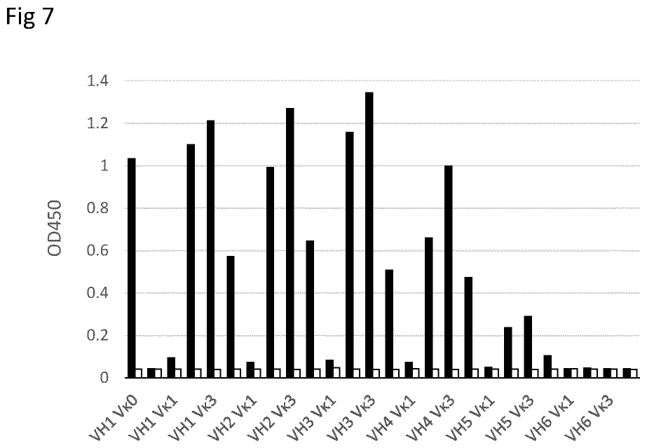

A total of six VH (SEQ ID NOS: 151-156) and four VK sequences (SEQ ID NOS: 157-160) were designed (summarised in FIG. 6) and expressed in all possible combinations to create 24 new humanised variants. When expressed in mammalian cells, variants containing VH1, VH2, VH3, VH4 or VH5 form antibodies that bind to full length recombinant 2N4R tau (FIG. 7). Background ELISA signal is low, demonstrating that antibody-related signal is due to a specific interaction with tau. Expression levels of the humanised variants are variable following transient transfection in HEK293 cells (see Table 10), so ELISA signal obtained from a single dilution of supernatant does not provide an indication of binding affinity.

Biacore single cycle kinetics (SCK) experiments enable calculation of antibody K_{D}. For the humanised variants, Kos range from 3.2 nM to 14.2 nM (for VH1VK1 and VH5VK3 respectively; summarised in Table 10). Variants containing VH6 do not bind to 2N4R tau (based on ELISA and Biacore data) indicating that residues mutated in this sequence may be required for binding of the parental antibody to tau. Specifically, VH6 includes a mutation within VH CDR1 (A35S; numbered according to Kabat) which may be necessary for binding to tau.

Data demonstrate that humanised variants of clone #44 that retain the original parental CDR sequences, retain high affinity binding to tau (<20 nM). It is predicted that the binding affinity of these antibodies would be equivalent to any tau species containing the epitope corresponding to amino acids 369-381 of 2N4R tau (SEQ ID NO: 1), if this sequence is accessible. This sequence (SEQ ID NO: 1) is 100% conserved within mammalian species so it is predicted that activity described for human tau, will be applicable to other mammalian species of tau. As the CDR sequences of the humanised variants are identical to the parental antibody, and binding to 2N4R tau is equivalent, it is predicted that the CDR-driven biological activity of the new variants will be equivalent to the parental clone #44.

21.1 Design of Composite Human Antibody variable regions: Structural models of the antibody V regions were produced using Swiss PDB (Guex & Peitsch, Electrophoresis 18, 2714-2722, 1997) and analysed in order to identify important “constraining” amino acids in the V regions that were likely to be essential for the binding properties of the antibody. Most residues contained within the CDRs (using both Kabat and Chothia definitions) together with a number of framework residues were considered to be important.

When compared to human antibodies, the first amino acid of the rabbit heavy chain is absent in clone #44 (in common with the majority of rabbit germline VH genes). It also contains a two amino acid deletion within VH FW3 that is found in a subset of rabbit germline genes (FIG. 6).

Based on this analysis, composite Human sequences were created with a wide latitude for alternative residues outside of the CDRs but with only a narrow menu of possible residues within the CDR sequences. Preliminary analysis indicated that corresponding sequence segments from several human antibodies could be combined to create CDRs similar or identical to those in the rabbit sequences. For regions outside of, and flanking the CDRs, a wide selection of human sequence segments was identified as possible components of the novel humanised V regions.

21.2 CD4+ T cell epitope avoidance (analysis by iTope™): Based upon the structural analysis, a large preliminary set of sequence segments were identified that could be used to create humanised variants. These segments were selected and analysed using iTope™ technology for in silico analysis of peptide binding to human MHC class II alleles (Perry et al, Drugs R D 9 (6): 385-96, 2008). The iTope™ software predicts favourable interactions between amino acid side chains of a peptide and specific binding pockets of 34 human MHC class II alleles. These alleles represent the most common HLA-DR alleles found world-wide with no weighting attributed to those found most prevalently in any particular ethnic population. The location of key binding residues is achieved by the in silico generation of 9mer peptides that overlap by eight amino acids spanning the test protein sequence.

Selected sequence segments identified as having a reduced risk of MHC class II binding were assembled into complete V region sequences with reduced T cell epitopes. Variant sequences are shown in FIG. 6.

21.3 Generation of humanised antibody variants: New humanised variants of hIgG1 were generated by Abzena (Cambridge, UK) using proprietary methods. Briefly, DNA encoding variable regions for Composite Human Antibodies were synthesized, cloned onto an expression vector with human constant regions (hIgG1) and transiently transfected into HEK293 cells. Supernatants containing hIgG1 were collected and analysed.

21.4 Assessment of antibody binding to tau (ELISA): ELISA plates were coated with full length 2N4R tau (SEQ ID NO: 2), 100 ng/well; or 1% BSA in 1×TBS) in 1× carbonate-bicarbonate buffer for 1 hour at 37° C. Antigen was removed from wells and the plates were then blocked for 1 hour at RT with 5% dried milk in 1×TBS. Blocking solution was removed, HEK293 cell supernatant (1:100 in 1% BSA/1×TBS) was added to relevant wells, and plates were incubated for 1 hour at RT with gentle shaking. Plates were then washed four times with TBS/0.1% Tween (TBST). Goat anti-human IgG-HRP antibody (Thermo Fisher Scientific, Waltham, MA, USA), diluted 1:2000 in 5% milk/TBS, was added to each well and plates were incubated for 1 hour at RT with gentle shaking before being washed four times with TBST. 3,3′,5,5′-tetramethylbenzidine (TMB) ELISA solution was added to each well and plates were incubated for 15 mins at RT. An equal volume of 1 M sulfuric acid was added to each well and OD was measured at 450 nm.

21.5 Assessment of antibody binding to tau (Biacore SCK analysis): Binding of anti-tau hIgG1 to full length recombinant 2N4R tau (Rpeptide; SEQ ID NO: 2) was assessed using the Biacore T200 (GE Healthcare, Chicago, IL, USA) running Biacore T200 Evaluation Software V2.0.1. hIgG1 were immobilised on a Protein A capture sensor chip in running buffer (HBS-EP+ buffer containing 1 mg/mL BSA) at 25° C., captured to ˜50 RU at 10 μL/min. For single-cycle kinetics experiments, recombinant 2N4R tau was flowed at concentrations ranging from 1.25 nM to 10 nM (2-fold dilutions) with an association time of 60 s and a dissociation time of 200 s. Curves were compared to a reference cell that was mock immobilized (no antibody present).

Data were analysed using Langmuir (1:1) binding analysis, as described in Example 19.2.

TABLE 10
Summary of binding analysis data for humanised variants of clone #44 (SCK) binding
to full length recombinant 2N4R tau. The parental clone #44 is shown as VH0VK0 (SEQ
ID NO: 118 and 119), and ‘relative K_{D}’ values are calculated relative to
this clone. HEK titres are based on Octet analysis at Day 7 post-transfection.

| | | | | Relative | | Chi² | HEK Titer |
|---|---|---|---|---|---|---|---|
| Antibody | kₐ (1/Ms) | k_{d} (1/s) | K_{D} (M) | K_{D} | R_{MAX} | (RU²) | (mg/mL) |
| VH0Vκ0 | 3.28E+06 | 1.26E−02 | 3.83E−09 | 1 | 24.7 | 0.0863 | 3.23 |
| VH1Vκ1 | 7.35E+06 | 2.32E−02 | 3.16E−09 | 0.83 | 8.9 | 0.0404 | <1.0 |
| VH1Vκ2 | 5.43E+06 | 2.32E−02 | 4.26E−09 | 1.11 | 32 | 0.143 | 3.94 |
| VH1Vκ3 | 2.58E+06 | 1.29E−02 | 5.00E−09 | 1.31 | 33.8 | 0.11 | 4.26 |
| VH1Vκ4 | 5.01E+06 | 2.08E−02 | 4.15E−09 | 1.08 | 34.8 | 0.253 | 1.87 |
| VH2Vκ1 | 2.64E+06 | 2.10E−02 | 7.94E−09 | 2.07 | 13.5 | 0.0771 | <1.0 |
| VH2Vκ2 | 3.63E+06 | 1.82E−02 | 5.00E−09 | 1.31 | 30 | 0.0945 | 4.04 |
| VH2Vκ3 | 4.14E+06 | 1.87E−02 | 4.52E−09 | 1.18 | 25.7 | 0.153 | 3.95 |
| VH2Vκ4 | 3.66E+06 | 1.95E−02 | 5.33E−09 | 1.39 | 39.8 | 0.17 | 1.5 |
| VH3Vκ1 | 2.79E+06 | 1.21E−02 | 4.35E−09 | 1.14 | 7.2 | 0.0288 | <1.0 |
| VH3Vκ2 | 4.66E+06 | 3.56E−02 | 7.64E−09 | 1.99 | 29.6 | 0.192 | 4.89 |
| VH3Vκ3 | 4.43E+06 | 3.41E−02 | 7.71E−09 | 2.01 | 30.9 | 0.156 | 5.52 |
| VH3Vκ4 | 6.12E+06 | 4.30E−02 | 7.02E−09 | 1.83 | 32.7 | 0.335 | 1.68 |
| VH4Vκ1 | 4.40E+06 | 3.00E−02 | 6.81E−09 | 1.78 | 6.2 | 0.0317 | <1.0 |
| VH4Vκ2 | 3.60E+06 | 2.47E−02 | 6.86E−09 | 1.79 | 25.2 | 0.0904 | 2.89 |
| VH4Vκ3 | 5.09E+06 | 3.40E−02 | 6.68E−09 | 1.74 | 30.7 | 0.243 | — |
| VH4Vκ4 | 3.61E+06 | 2.94E−02 | 8.12E−09 | 2.12 | 35.7 | 0.171 | 1.58 |
| VH5Vκ1 | 2.25E+07 | 1.87E−01 | 8.31E−09 | 2.17 | 7.4 | 0.0875 | <1.0 |
| VH5Vκ2 | 1.47E+09 | 1.85E+01 | 1.26E−08 | 3.29 | 19.7 | 0.244 | 4.52 |
| VH5Vκ3 | 2.86E+06 | 4.06E−02 | 1.42E−08 | 3.71 | 20.4 | 0.219 | 5.05 |
| VH5Vκ4 | 7.11E+06 | 6.84E−02 | 9.63E−09 | 2.51 | 14.9 | 0.257 | 1.81 |
| VH6Vκ1 | — | — | — | — | — | — | <1.0 |
| VH6Vκ2 | — | — | — | — | — | — | 0.97 |
| VH6Vκ3 | — | — | — | — | — | — | 1.25 |
| VH6Vκ4 | — | — | — | — | — | — | <1.0 |

### Example 22. Humanised Anti-Tau IgG Bind to Tau with High Affinity

The top five humanised variants were selected for larger scale expression and further characterisation (based on data summarised in Table 10): VH3VK3 (SEQ ID NOS: 153 and 159); VH3VK4 (SEQ ID NOS: 153 and 160); VH4VK2 (SEQ ID NOS: 154 and 158); VH4VK3 (SEQ ID NOS: 154 and 159); VH4VK4 (SEQ ID NOS: 154 and 160). Yields obtained from transfections in HEK293 cells were lower than expected, so CHO cells were used for this larger scale production.

Biacore multi-cycle kinetics (MCK) analyses for antibody binding to recombinant 2N4R tau are summarised in FIG. 8 and Table 11. All five variants demonstrate a K_{D} within 2-fold of the chimeric parent antibody (VH0VK0; K_{D}=3.25 nM). Slight variations in R_{MAX} are considered likely to be due to differences in ligand capture and not reflective of significant variation in antibody binding characteristics.

Data confirm that humanised variants retain the binding characteristics of the parental (rabbit) antibody (VH0VK0), clone #44, to full length 2N4R tau. As described in Example 21, it is predicted that activity described for human tau, will be applicable to other mammalian species of tau, if the target sequence (SEQ ID NO: 1) is present and accessible.

22.1 Production of Protein A purified hIgG1 antibodies: Clone #44 VH0VK0 and new humanised variants of hIgG1 were generated by Abzena (Cambridge, UK) using proprietary methods. Briefly, DNA encoding variable regions for Composite Human Antibodies were synthesized, cloned onto an expression vector with human constant regions (hIgG1) and transiently transfected into CHO cells. Supernatants containing hIgG1 were collected and hIgG1 were affinity purified, buffer exchanged into phosphate buffered saline, sterile filtered, then further purified by size exclusion chromatography (SEC) to achieve a final monomer purify of >99%.

22.2 Assessment of antibody binding to tau (Biacore MCK analysis): Binding of anti-tau hIgG1 to full length recombinant 2N4R tau (Rpeptide; SEQ ID NO: 2) was assessed using the Biacore T200 (GE Healthcare, Chicago, IL, USA) running Biacore T200 Evaluation Software V2.0.1. hIgG1 were immobilised on a Protein A capture sensor chip in running buffer (HBS-EP+ buffer containing 1 mg/mL BSA) at 25° C., captured to ˜50 RU at 10 L/min. For multi-cycle kinetics experiments, recombinant 2N4R tau was flowed at concentrations ranging from 0.39 nM to 12.5 nM in running buffer at 40 μL/min, with an association time of 60 s and a dissociation time of 200 s. Curves were compared to a reference cell that was mock immobilized (no antibody present).

Data were analysed using Langmuir (1:1) binding analysis, as described in Example 19.2.

TABLE 11
Summary of Biacore MCK analysis for purified humanised variants of clone #44
binding to full length recombinant 2N4R tau. The parental clone #44 is shown as VH0VK0,
and ‘relative K_{D}’ values are calculated relative to this clone. Humanised variants were tested
as SEC purified samples. The parental IgG was tested as a HEK293 supernatant.

| | | | | | | | Ligand |
|---|---|---|---|---|---|---|---|
| | | | | Relative | Rₘₐₓ | Chi² | Level |
| Ligand | kₐ (1/Ms) | k_{d} (1/s) | K_{D} (M) | K_{D} | (RU) | (RU²) | (RU) |
| #44 VH0Vκ0 sup | 3.30E+06 | 1.07E−02 | 3.25E−09 | 1 | 26.8 | 0.104 | 82.7 |
| #44 VH3Vκ3 SEC | 1.63E+07 | 9.27E−02 | 5.70E−09 | 1.75 | 16 | 0.19 | 71.1 |
| #44 VH3Vκ4 SEC | 9.40E+06 | 6.18E−02 | 6.57E−09 | 2.02 | 18.5 | 0.151 | 78.8 |
| #44 VH4Vκ2 SEC | 9.14E+06 | 4.01E−02 | 4.39E−09 | 1.35 | 15.5 | 0.12 | 69.1 |
| #44 VH4Vκ3 SEC | 7.83E+06 | 4.03E−02 | 5.14E−09 | 1.58 | 16.7 | 0.138 | 69.1 |
| #44 VH4Vκ4 SEC | 5.41E+06 | 3.16E−02 | 5.84E−09 | 1.8 | 24.9 | 0.165 | 81.5 |

### Example 23. Humanised IgG Variants are Thermodynamically Stable

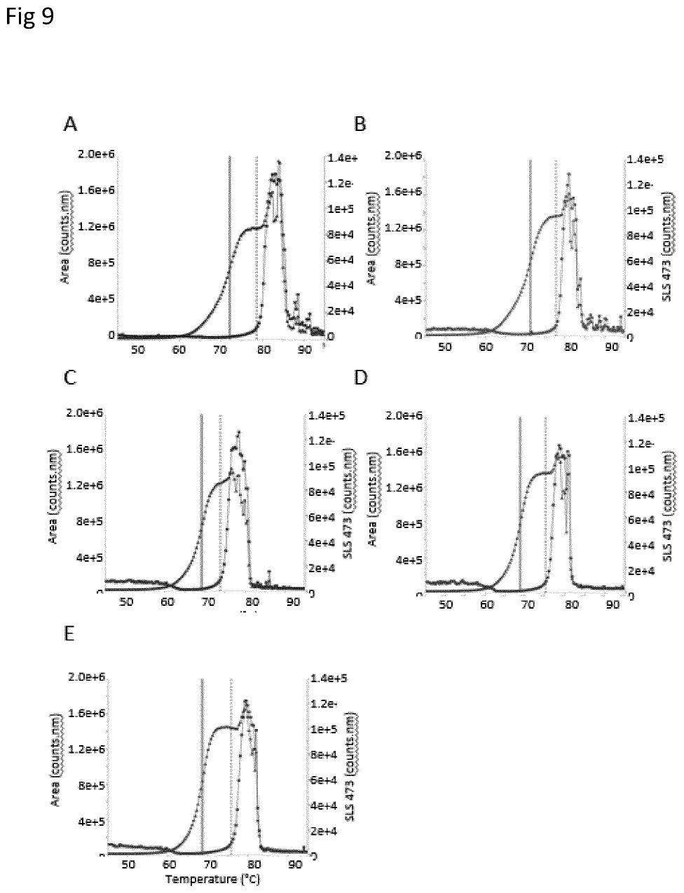

To assess the potential of humanised variants for development into therapeutic antibodies, the thermal stability of each was assessed. Data are summarised in Table 12 and FIG. 9. Average melting temperature (Tm) ranged from 68.7° C. for VH4VK4 to 71.9° C. for VH3VK3, which is considered acceptable for a therapeutic antibody.

23.1 Thermal stability analysis: Thermal stability was assessed using SYPRO orange (Lo et al, Analytical Biochem 332 (1): 153-9, 2004). Samples were prepared as indicated in Table 13. 9 μL of each sample mixture was loaded in duplicate into Uncle Uni microcuvettes and run with the ‘Tm using SYPRO’ application. Samples were subjected to a thermal ramp from 15-95° C., with a ramp rate of 0.3° C./min and excitation at 473 nm. Full spectra were collected from 250-720 nm and Uncle software used the area under the curve between 510-680 nm to calculate the infection points of the transition curves. Monitoring static light scattering (SLS) at 473 nm allows the detection of protein aggregation in the same experiment. Onset of aggregation (Tagg) was calculated from the resulting SLS profiles.

TABLE 12
Summary of thermal stability profiling data for humanised variants
of clone #44. Parental clone #44 is shown as VH0VK0.

| | | Average | | Average | Tagg | Average |
|---|---|---|---|---|---|---|
| | Tm1 | Tm1 | Tonset | Tonset | 473 | Tagg 473 |
| Sample | (° C.) | (° C.) | (° C.) | (° C.) | (° C.) | (° C.) |
| #44 VH3Vκ3 | 71.69 | 71.9 | 61.74 | 61.8 | 78.14 | 78.4 |
| | 72.1 | | 61.85 | | 78.62 | |
| #44 VH3Vκ4 | 71.39 | 71.4 | 61.56 | 61.4 | 77.8 | 77.8 |
| | 71.32 | | 61.22 | | 77.71 | |
| #44 VH4Vκ2 | 69.21 | 69.2 | 61.42 | 61.6 | 73.82 | 74.1 |
| | 69.1 | | 61.72 | | 74.36 | |
| #44 VH4Vκ3 | 68.75 | 68.8 | 61.62 | 61.5 | 75.19 | 75.2 |
| | 68.75 | | 61.46 | | 75.14 | |
| #44 VH4Vκ4 | 68.67 | 68.7 | 61.98 | 61.9 | 75.84 | 75.2 |
| | 68.73 | | 61.78 | | 74.58 | |

TABLE 13
Details of sample preparation for thermal
stability analysis (Example 23.1).

| | | | Ab | | | |
|---|---|---|---|---|---|---|
| | | | 0.75 | | Sypro | |
| Ab | Variants | mg/mL | mg/mL | PBS | @ 80X | Total |
| #44 | VH3Vκ3 | 2.11 | 21.3 | 23.7 | 15 | 60 |
| #44 | VH3Vκ4 | 2.22 | 20.3 | 24.7 | 15 | 60 |
| #44 | VH4Vκ2 | 1.78 | 25.3 | 19.7 | 15 | 60 |
| #44 | VH4Vκ3 | 1.97 | 22.9 | 22.1 | 15 | 60 |
| #44 | VH4Vκ4 | 1.66 | 27 | 18 | 15 | 60 |

| | | | | | |
|---|---|---|---|---|---|
| Blank PBS | 0 | 0 | 45 | 15 | 60 |

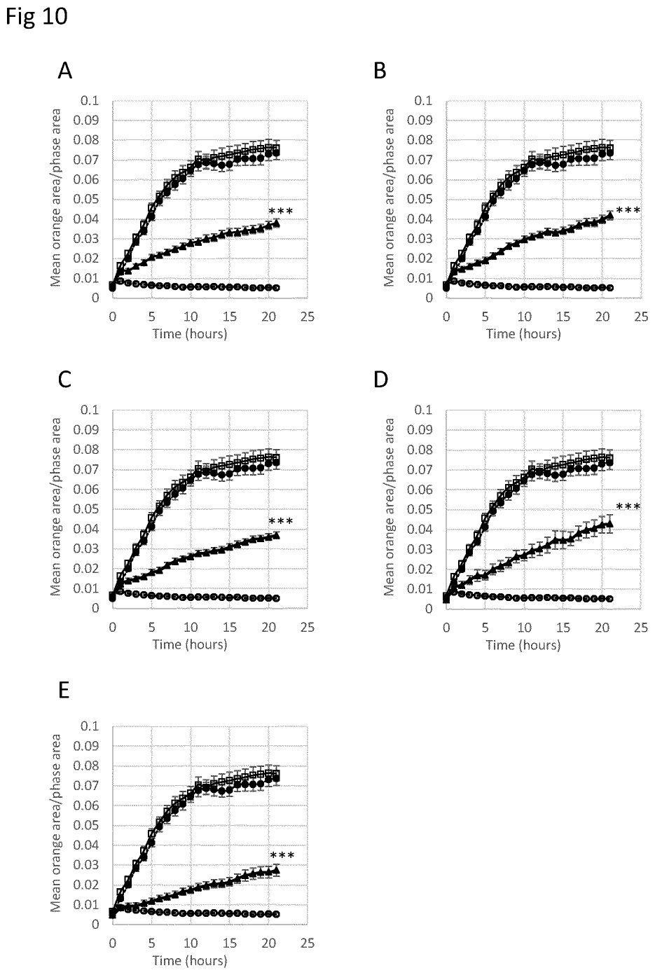

### Example 24. Humanised Variants of Anti-Tau Clone #44 Inhibit Uptake of Monomeric and Aggregated Tau into Human iPSC-Derived Neurons (FIG. 10, 11)

Neuronal uptake of ‘toxic’ forms of extracellular tau is proposed to play an important role in the pathogenic spreading of tau observed in tauopathies such as Alzheimer's disease. Extracellular monomeric and aggregated tau is taken up by human neurons via a combination of endocytosis and macropinocytosis (Evans et al. (2018) Cell Rep 22 (13): 3612-3624). This process occurs physiologically, but is also proposed to play a role in the pathogenic spreading of toxic forms of tau observed in tauopathies, including Alzheimer's disease. Inhibiting uptake of toxic tau species is therefore predicted to be therapeutically beneficial in limiting the spread of tau pathology in the brain. Neuronal uptake of tau can be assessed and quantified by measuring fluorescence associated with tau labelled with the pH-sensitive dye, pHrodo. Increased fluorescence occurs following internalisation of labelled tau into the acidic endosome compartment, thereby providing a dynamic measure of tau uptake/internalisation. Anti-tau rabbit IgG targeting SEQ ID NO: 1, including antibody clone #44 (Clone 2) are able to reduce uptake of tau species containing this epitope by human neurons (Example 12). Antibodies exhibiting this activity would be predicted to limit the neuron-neuron propagation of extracellular tau species in vivo and therefore to be therapeutically useful.

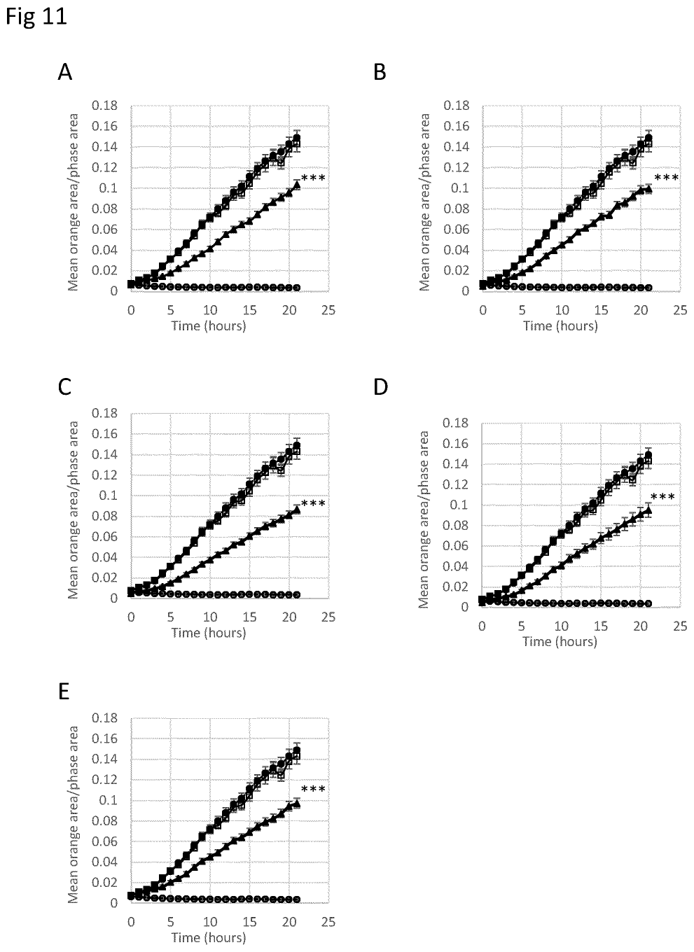

All humanised variants of clone #44 tested, significantly (P<0.001) inhibit uptake of monomeric tau into human iPSC-derived neurons (FIG. 10) to a similar extent to the rabbit clone: by 48.4±2.9% (VH3VK3), 43.1±3% (VH3VK4), 50±2.2% (VH4VK2), 41.8±6.3% (VH4VK3), 62.8±4.1% (VH4VK4). All humanised variants tested, also significantly (P<0.001) inhibit uptake of aggregated tau into human iPSC-derived neurons (FIG. 11) to a similar extent to the rabbit clone: by 30.6±3.2% (VH3VK3), 33.3±2.9% (VH3VK4), 41.9±2.8% (VH4VK2), 36.3±4.7% (VH4VK3), 34.9±3.2% (VH4VK4). Isotype control human IgG1 antibody had no significant effect on tau uptake in this system (inhibition of −3.4±5.2% and −1.2±5% for monomeric and aggregated tau respectively).

Data demonstrated that, like the parental rabbit antibody (#44, Clone 2), humanised antibodies targeting the amino acid sequence of SEQ ID NO: 1 were able to reduce the uptake of tau species containing this epitope, by human neurons. Such antibodies would therefore be predicted to limit the neuron-to-neuron propagation of extracellular tau species (both monomeric and multimeric) that include this epitope formed by the amino acid sequence of (SEQ ID NO: 1) in Alzheimer's disease and tauopathies, and thereby reduce/slow the progression of clinical symptoms in patients.

24.1 Production of human iPSC-derived cerebral cortex neurons: See Example 1.1.

24.2 Generation of aggregated (oligomeric) tau species: Tau P301S_10×his-tag_avi-tag was overexpressed in BL21 (DE3) bacteria. Cells were lysed using BugBuster (Millipore, Burlington, MA, USA) and clarified lysate was applied to a 5 mL HisTrapHP column (GE Healthcare, Chicago, IL, USA) in 2×PBS. Tau was eluted using a 0- to 500-mM imidazole gradient. Peak fractions were pooled and further purified in 2×PBS using a Superdex 200 16/60 gel filtration column (GE Healthcare, Chicago, IL, USA). Pooled fractions were then concentrated to approximately 8 mg/mL using a spin concentrator (Millipore, Burlington, MA, USA). Final protein concentration was determined by Nanodrop analysis.

1 mL tau P301S at 8 mg/mL was incubated with 4 mg/mL heparin (Sigma, St Louis, MO, USA) in PBS/30 mM 3-(N-morpholino) propanesulfonic acid (MOPS) (pH 7.2) at 37° C. for 72 h. Aggregated material was diluted in 9 mL PBS plus 1% (v/v) sarkosyl (Sigma, St Louis, MO, USA) and left rocking for 1 h at RT to completely solubilize any non-aggregated material. Insoluble tau was pelleted by ultracentrifugation for 1 h at 4° C. The pellet was resuspended in 1 mL PBS and sonicated at 100 W for 3×20 s (Hielscher UP200St ultrasonicator; Teltow, Germany) to disperse clumps or protein and break large filaments into smaller species.

24.3 Labelling of purified recombinant tau: Monomeric recombinant 2N4R tau was purchased from rPeptide (Watkinsville, GA, USA). Aggregated tau was prepared as described above. Recombinant monomeric tau (150 μM) or equivalent aggregated tau concentration (˜7 μg/mL) was incubated with 1.5 mM pHrodo Red Maleimide (dissolved in DMSO) and 1.5 mM tris(2-carboxyethyl) phosphine (1:10:10 molar ratio respectively) for 2 h in the dark at RT. Labelled samples were then subjected to size exclusion chromatography at 4° C. (Superdex 200 Increase 10/300 GL; GE Healthcare, Chicago, IL, USA) in 50 mM phosphate (pH 7.4) and 150 mM NaCl to remove unreacted dye. Oligomeric state of aggregates was assessed and found to be unaffected by labelling.

P301S tau was used for both monomeric and aggregated tau preparations.

24.4 Quantification of tau uptake by human iPSC-derived cortical neurons: Monomeric tau (25 nM) and aggregated tau (50 nM) were prepared in N2B27 (Thermo Fisher Scientific, Waltham, MA, USA) and incubated with a 10-fold molar excess of antibody over tau (i.e. 250 and 500 nM IgG) for 90 min at 37° C. Humanised variants of clone #44 were compared to an isotype control human IgG1 (anti-fluorescein [Apr. 4, 2020 (enhanced)], Absolute Antibody, Oxford, UK). 200 μL antibody/tau mix was added to NDC neurons (day 60+) and images were taken (fluorescence and bright field) every hour for 21 h at 37° C./5% CO₂ from 9 fields per well using the Incucyte S3 imaging system (Sartorius, Göttingen, Germany). Algorithms to quantify (per well) the mean area of fluorescence in the orange channel (excitation: 513-568 nm) were normalised to the mean area occupied by cells (phase area), and this was plotted as mean+/−SEM from 4 wells over time. One-way ANOVA with Tukey's multiple comparison test was run vs no antibody control to determine significance.

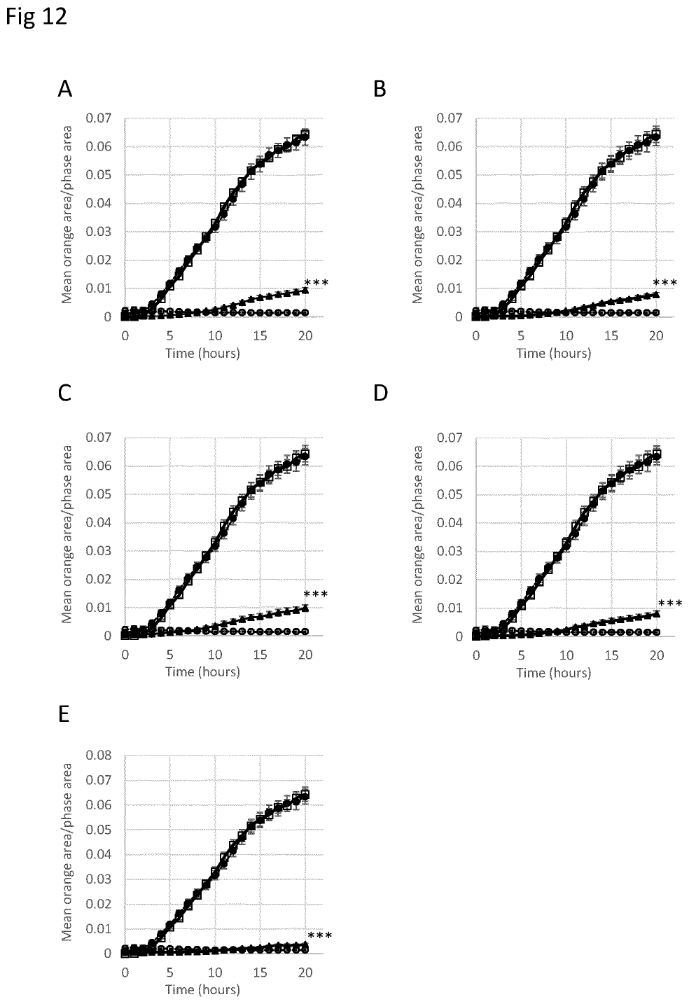

### Example 25. Humanised Variants of Anti-Tau IgG Clone #44 Inhibit Uptake of Monomeric and Aggregated Tau into Human Astrocytes (FIG. 12, 13)

Astrocytic uptake of extracellular tau is proposed to play a role in the pathogenic spreading of tau observed in tauopathies such as Alzheimer's disease.

Limited information is available on the uptake of extracellular tau species by human astrocytes, although this is known to occur in rodents (Martini-Stoica et al. J Exp Med 215 (9): 2355-2377 (2018)). In addition, a recently described receptor for neuronal tau uptake, lipoprotein receptor-related protein 1 (LRP1), is reported to be expressed in astrocytes (Rauch et al. Nature 580(7803):381-385 (2020)), suggesting that the mechanisms of uptake may be shared. As a major cell type in the central nervous system, with putative roles in the propagation of tau pathology in Alzheimer's disease and tauopathy (reviewed in Sidoryk-Węgrzynowicz & Strużyńska Biochem J 476 (22): 3493-3504 (2019)) we explored whether antibodies targeting the sequence corresponding to amino acids 369 to 381 of 2N4R tau (SEQ ID NO: 1) have any impact on uptake of tau species by astrocytes.

Human iPSC-derived astrocytes readily take up both monomeric and aggregated tau species. Anti-tau rabbit IgG targeting SEQ ID NO: 1, including antibody clone #44 (Clone 2) are able to reduce uptake of tau species containing this epitope by human astrocytes. Incubation of tau with anti-tau rabbit clone #44, significantly (P<0.001) inhibited uptake of monomeric 2N4R tau by 98.4±0.4% and of aggregated tau by 43.3±2.9% compared to uptake in the absence of antibody. This data provided evidence that therapeutic anti-tau antibodies targeting SEQ ID NO: 1, would reduce the uptake of toxic forms of tau by astrocytes, and thereby reduce the impact of astrocytes in the propagation of tau pathology. This activity is predicted to be therapeutically beneficial.

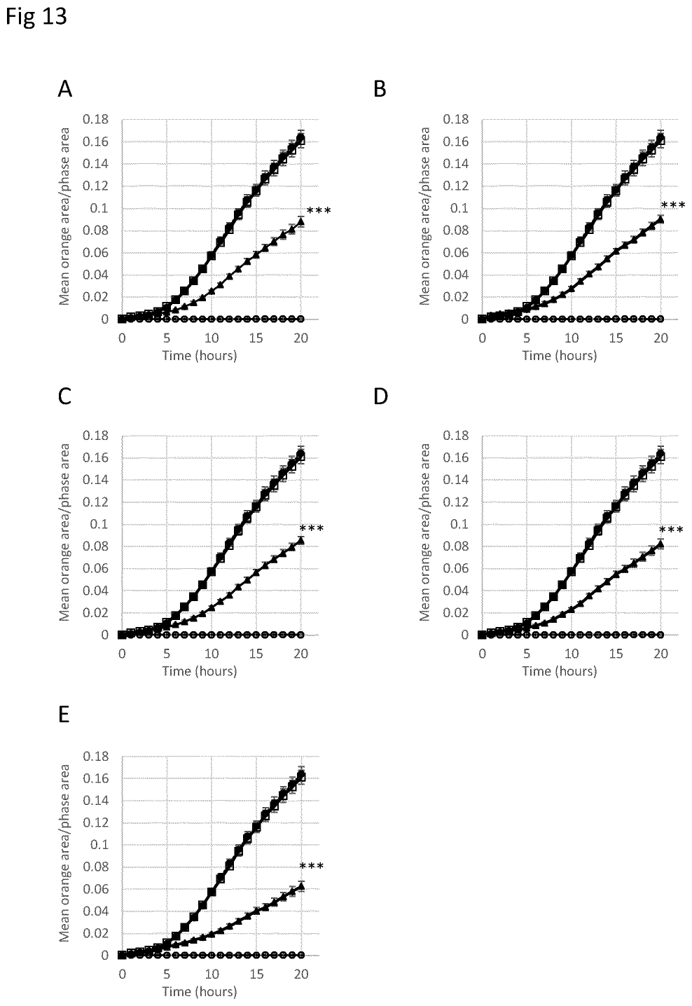

All humanised variants of clone #44 tested, significantly (P<0.001) inhibited uptake of monomeric tau into human iPSC-derived astrocytes (FIG. 12) to a similar extent to the rabbit clone: by 85.2±1.4% (VH3VK3), 87.6±1.2% (VH3VK4), 84.6±1.9% (VH4VK2), 87.5±1.7% (VH4VK3), 94.1±0.6% (VH4VK4). All humanised variants tested, also significantly (P<0.001) inhibited uptake of aggregated tau into human iPSC-derived astrocytes (FIG. 13) to a similar extent to the rabbit clone: by 46.4±3.9% (VH3VK3), 45.0±2.8% (VH3VK4), 48.0±2.1% (VH4VK2), 49.9±2.2% (VH4VK3), 61.9±2.8% (VH4VK4). Isotype control human IgG1 (anti-fluorescein [Apr. 4, 2020 (enhanced)], Absolute Antibody, Oxford, UK) had no significant effect on tau uptake in this system (inhibition of −1.6±4.5% and 1.8±3.9% for monomeric and aggregated tau respectively).

Data demonstrated that, like the parental rabbit antibody (#44, Clone 2), humanised antibodies targeting epitopes formed by SEQ ID NO: 1 were able to reduce the uptake of tau species containing this epitope, by human astrocytes. This activity is predicted to be therapeutically beneficial.

25.1 Production of human iPSC-derived astrocytes: Differentiation of human iPSC to astrocytes was carried out using iPSC lines from an NDC background. Neuroepithelial sheets were generated as described for cortical neurons (Shi et al., Nature Protocols 7 (10): 1836-46, 2012; protocol followed to step 31). From day 16, cells were passaged with Accutase into new Matrigel-coated plates (1.5×10⁶ cells/well of a 6 well plate) and transferred into ‘Astrocyte differentiation media 1’ (neural maintenance media described in Shi et al., Nature Protocols 7(10): 1836-46, 2012; supplemented with 20 ng/ml FGF2, 20 ng/ml EGF) for 7 days, with media changes every other day. Cells were then passaged with Accutase into new Matrigel-coated plates (as before) and transferred into ‘Astrocyte differentiation media 2’ (Neural maintenance media supplemented with 10 ng/ml BDNF, 10 ng/ml CNTF, 1 UM purmorphamine) for 7 days, with media changes every other day. Astrocytes were then maintained in ‘maturation media’ (Neurobasal media, 1×B27 supplement, 1% FBS, 50 U/mL penicillin and 50 mg/mL streptomycin, 1× GlutaMAX) until use (at ˜day 130+). 25.2 Generation of aggregated (oligomeric) tau species: See Example 24.2

25.3 Labelling of purified recombinant tau: See Example 24.3.

P301S tau was used for both monomeric and aggregated tau preparations.

25.4 Quantification of tau uptake by human iPSC-derived astrocytes: Monomeric tau (25 nM) and aggregated tau (50 nM) were prepared in serum-free Optimem (ThermoFisher) media and incubated with tested antibodies at a 10-fold molar excess concentration (i.e. 250 and 500 nM IgG respectively) for 90 min at 37° C. 200 μL antibody/tau mix was added to iPSC-derived astrocytes and images were taken (bright field and orange channel) every hour for 20 h at 37° C./5% CO₂ from 9 fields per well using the Incucyte S3 imaging system (Sartorius, Göttingen, Germany). Algorithms to quantify (per well) the mean area of fluorescence in the orange channel (excitation: 513-568 nm) were normalised to the mean area occupied by cells (phase area), and this was plotted as mean+/−SEM from 4 wells over time. One-way ANOVA with Tukey's multiple comparison test was run vs no antibody control to determine significance.

In this experiment, an anti-human IgG1 isotype control was used (anti-fluorescein [Apr. 4, 2020 (enhanced)], Absolute Antibody, Oxford, UK).

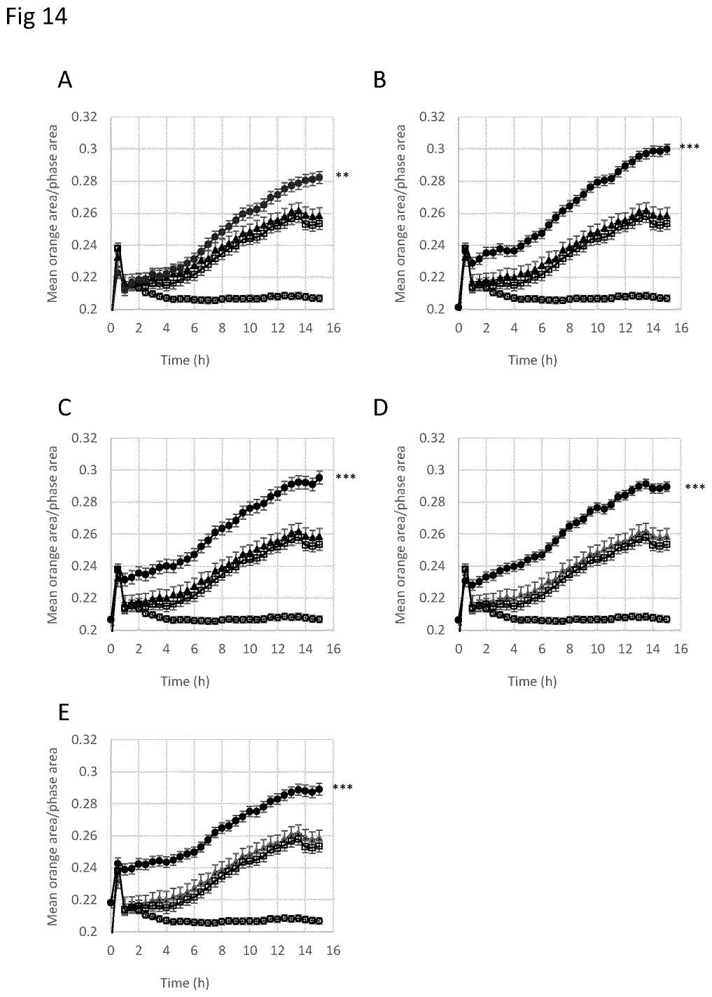

### Example 26. Humanised Variants of Monoclonal Anti-Tau Clone #44 Human IgG1 Increase Uptake of Monomeric and Aggregated Tau by Human iPSC-Derived Microglia (FIG. 14, 15)

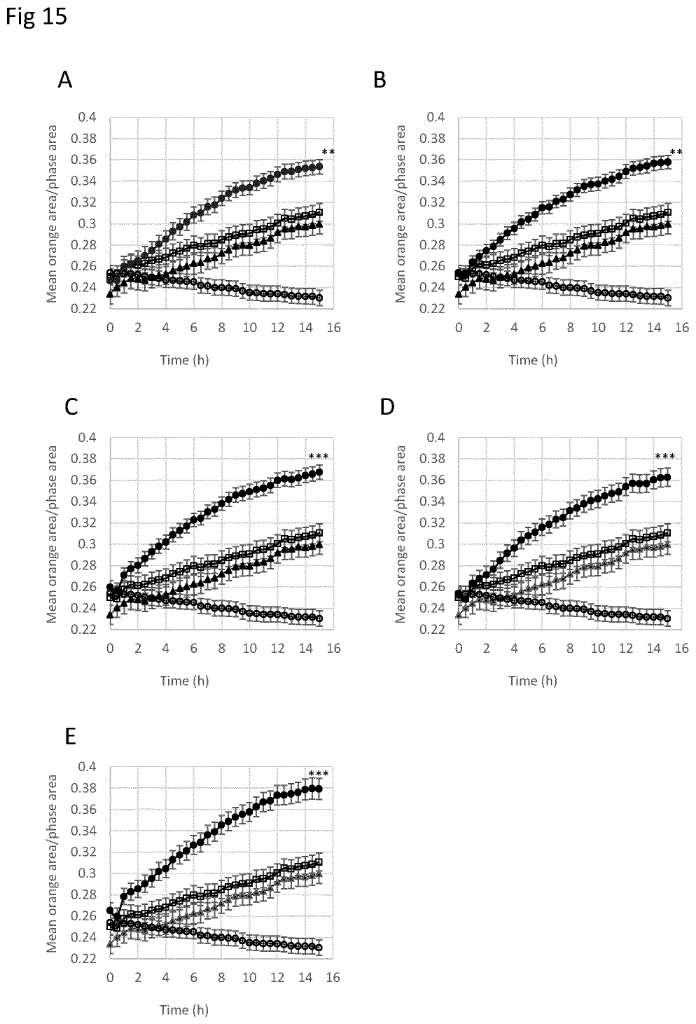

Microglia play an important role in clearing extracellular material in the central nervous system, to prevent accumulation of debris and enable repair processes to occur. In the context of neurodegenerative disease, phagocytosis of extracellular proteins, including aggregates, oligomers and monomeric forms, helps to reduce the extracellular concentrations of these species. All humanised variants of clone #44 tested as hIgG1, significantly (P<0.005) increased uptake of monomeric tau into human iPSC-derived microglia (FIG. 14): by 16.1±1% (VH3VK3), 80.3±0.8% (VH3VK4), 71.4±1.6% (VH4VK2), 60.2±1.1% (VH4VK3), 59.4±1.4% (VH4VK4). All humanised variants tested as hIgG1, also significantly (P<0.001) increased uptake of aggregated tau into human iPSC-derived microglia (FIG. 15): by 77.7±2.1% (VH3VK3), 83.9±2.4% (VH3VK4), 97.6±2.2% (VH4VK2), 90.5±2.8% (VH4VK3), 114.6±3.3% (VH4VK4). Isotype control antibody had no significant effect on tau uptake in this system (decrease of 9.4±1.5% and increase of 15.7±2.2% for monomeric and aggregated tau respectively).

Data demonstrated that, like the parental antibody (#44, Clone 2), therapeutic antibodies targeting SEQ ID NO: 1, with effector function (e.g., formatted as hIgG1) would increase clearance of extracellular tau containing the targeted sequence (SEQ ID NO: 1), by microglia, and thereby reduce the extracellular concentration and deleterious effects of extracellular tau in the CNS. This activity is predicted to be therapeutically beneficial.

26.1 Production of hIgG1 antibodies: See Example 22.1.

26.2 Production of human iPSC-derived microglia: Differentiation of human pluripotent stem cells (iPSC) to microglial cultures was carried out as described by Brownjohn et al. (2018) Stem Cell Rep 10 (4): 1294-1307. An iPSC line from an NDC background was used. Microglial progenitor cells were collected, plated in 96 well plates and maintained in complete microglia media (as described in Brownjohn et al., 2018) for approximately 14 days before use. On the day prior to use, cultures were switched into serum free media (RPMI 1640/Glutamax supplemented with 10 ng/mL GM-CSF and 100 ng/ml IL-34 (growth factors from Peprotech, NJ, US)) and phagocytosis experiments were completed in serum-free conditions.

26.3 Generation of aggregated (oligomeric) tau species: See Example 24.2

26.4 Labelling of purified recombinant tau: See Example 24.3. Note that P301S tau was used for both monomeric and aggregated tau preparations.

26.5 Quantification of tau uptake by human iPSC-derived microglia: Monomeric tau (25 nM) and aggregated tau (50 nM) were prepared in serum-free microglial media and incubated with a 1:10 ratio of tau:antibody (i.e. 2.5 and 5 nM IgG respectively) for 90 min at 37° C. Anti-tau hIgG1 was compared to an isotype control (anti-fluorescein [Apr. 4, 2020 (enhanced)], Absolute Antibody, Oxford, UK). 100 μL antibody/tau mix was added to iPSC-derived microglia and images were taken (bright field and orange channel) every 30 min for 15 h at 37° C./5% CO₂ from 9 fields per well using the Incucyte S3 imaging system (Sartorius, Göttingen, Germany). Algorithms to quantify (per well) the mean area of fluorescence in the orange channel (excitation: 513-568 nm), normalised to the mean area occupied by cells (phase area) and this was plotted as mean+/−SEM from n=4 cells over time. One-way ANOVA with Tukey's multiple comparison test was run vs no antibody control to determine significance. Increases in tau uptake quoted are calculated relative to the ‘no tau’ control.

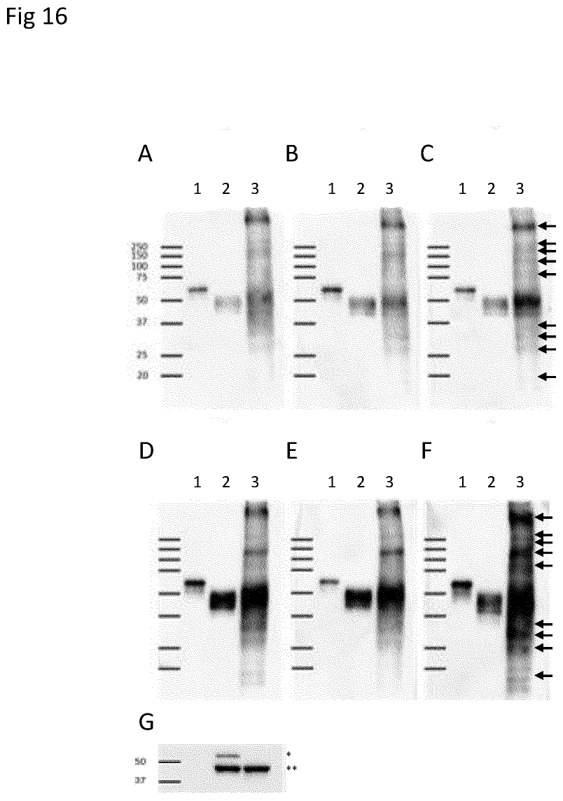

### Example 27. Humanised Variants of Monoclonal Anti-Tau Clone #44 Detect Increased Levels of High and Low MW Tau Species in Familial Alzheimer's Disease but not Non-Demented Control Postmortem Brain (FIG. 16)

Humanised variants of anti-tau antibody clone #44 detect disease-relevant forms of tau in postmortem familial Alzheimer's Disease (fAD; Presenilin 1 mutation) cerebral cortex samples but not in non-demented control (NDC) samples. Western blots demonstrated that five variants: VH3VK3, VH3VK4, VH4VK2, VH4VK3 and VH4VK4, detected increased levels of tau in a representative fAD compared to an NDC sample, including multiple high (>75 kD) and low (<40 kD) molecular weight species that wereabsent in the NDC samples tested (FIG. 16). Actin controls confirmed equal loading of brain lysate samples. Tau species detected show a similar pattern to the parental antibody (#44 VH0VK0) demonstrating that the binding characteristics of the parental rabbit clone #44 have been retained by the humanised variants.

27.1 Human brain samples: See Example 8.1 for details.

27.2. Protein extraction: iPSC-derived neuronal cultures were lysed using RIPA buffer (Sigma, St Louis, MO, USA) supplemented with protease inhibitors (complete Mini, EDTA free, Roche Diagnostics, Rotkreux, Switzerland). Protein concentration was measured with Pierce BCA Protein Assay Kit (Thermo Fisher Scientific, Waltham, MA, USA), and where specified, brain lysates were treated with lambda protein phosphatase (I-PP); (New England Biolabs, Ipswich, MA, USA), according to manufacturer's instructions.

27.3 Western blotting: 40 μg protein in 20 μL total volume (unless otherwise stated) were loaded on a 12% Mini-Protean TGX precast gel (Bio-Rad, Hercules, CA, USA) and transferred onto 0.2 μm PVDF membranes (GE Healthcare Life science, Chicago, IL, USA) at 200 mA for two hours at 4° C. Membranes were incubated in blocking solution (5% dried skimmed milk, 0.1% Tween in PBS) for 1 hour at RT.

27.4 Antibody incubation: The protein-transferred membranes were probed overnight at RT with the primary antibody (at the concentration specified). Membranes were subsequently incubated with Tidyblot (Bio-Rad, Hercules, CA, USA; 1:200) for 1 hour at RT. Tidyblot was used in place of a standard secondary antibody to minimise the influence of contaminating human IgG present in the postmortem brain samples

27.5 Membrane visualization: Each membrane was detected using enhanced chemiluminescence (ECL) western blotting detection reagent (GE Healthcare Life Science, Chicago, IL, USA) and visualized using ImageQuant LAS 4000 (GE Healthcare Life Science, Chicago, IL, USA).

27.6 Beta-Actin normalization: Beta-actin was included as a loading control. After imaging the first antibody complex was removed from PVDF membranes using Restore PLUS Western Blot Stripping Buffer (Thermo Fisher Scientific, Waltham, MA, USA) for 25 minutes at RT. The membranes were incubated with blocking solution for 1 hour at RT. Each membrane was probed with mouse monoclonal anti-beta-Actin (Sigma, St Louis MO, USA; 1:1000), or TuJ1 primary antibody (R&D Systems, Minneapolis, MN, USA; 1:1000) and then incubated with goat anti-mouse IgG-peroxidase secondary antibody (Sigma, St Louis, MO, USA; 1:2000). Both antibodies were incubated for 1 hour at RT consecutively.

### Example 28. Humanised Variant #44 VH4VK4 Detects Increased Levels of High and Low MW Tau Species in Familial Alzheimer's Disease, Sporadic Alzheimer's Disease and Dementia with Lewy Bodies Brain Compared to Non-Demented Control Brain

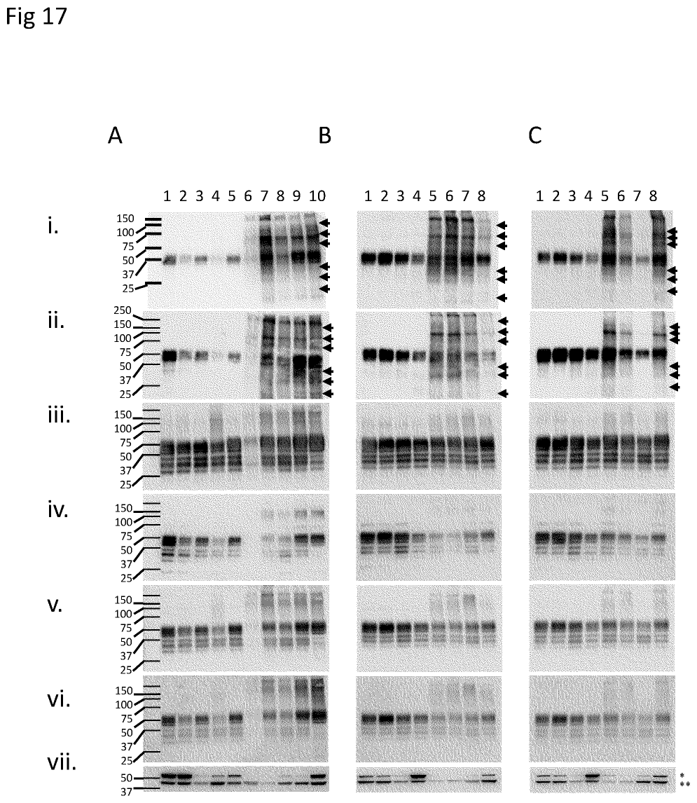

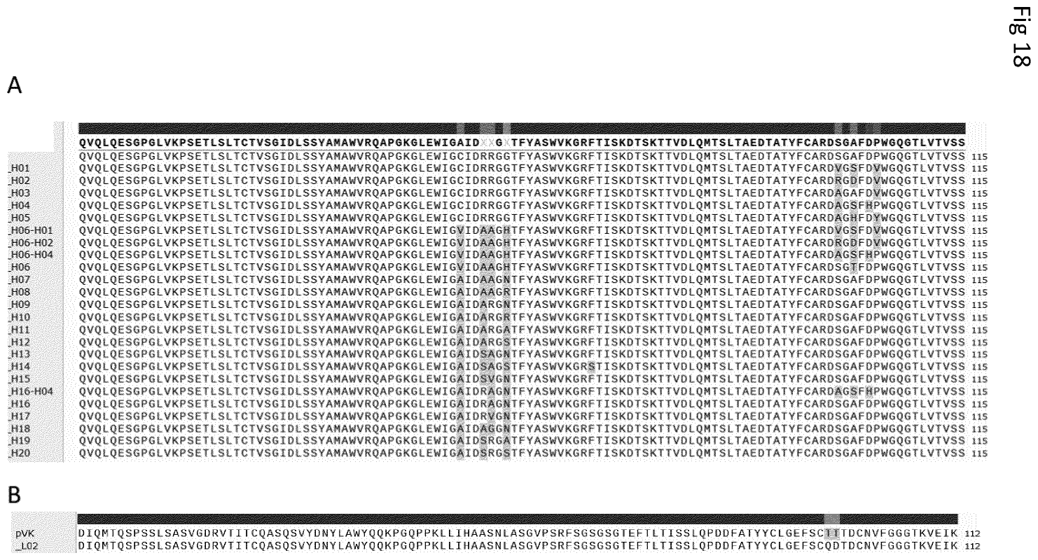

In order to confirm that humanised variants of #44 retained the ability of the parental rabbit IgG to detect disease-relevant tau species across a range of tauopathies and across a panel of patient samples, the parental clone #44 (rabbit IgG) and humanised variant #44 VH4VK4 (hIgG1) were profiled in more detail. As expected, humanised variant #44 VH4VK4 performed similarly to the parental clone #44 and detected increased levels of both high and low MW species across a panel of patient samples representing familial Alzheimer's disease (fAD), sporadic Alzheimer's disease (SAD) and Dementia with Lewy bodies (DLB) (FIG. 17). Actin and neuronal tubulin controls confirmed that changes in tau levels were not due to variations in protein and/or neuronal levels in the samples tested. Data confirmed that both the parental clone #44 and humanised antibody variant VH4VK4 detected disease-relevant tau species in a similar manner to that described for antibody clone #66 (clone 1), and suggested that the panel of humanised variants described in Example 21, as well as humanised variants of any other antibody binding to SEQ ID NO: 1, were likely to behave similarly. Binding of #44 VH4VK4, or alternative humanised variants, to disease-specific tau species therefore have the potential to be therapeutically useful in the treatment of AD and tauopathy.

In addition, detection of tau species by commercially available N-terminal (Tau13), mid-region (HT7 and Tau5) and far C-terminal tau (Tau46) antibodies showed limited detection of disease-specific tau species across fAD, SAD and DLB brain samples (FIG. 17), in both the higher and lower molecular weight range. Data demonstrated that antibodies targeting the sequence corresponding to amino acids 369-381 of 2N4R tau (SEQ ID NO: 1) bind to disease-specific tau species that are not detected by a range of antibodies targeting N-terminal, mid-region or far C-terminal regions of tau, and therefore show unique and beneficial properties related to the targeted sequence.

28.1 Human brain samples: See Example 8.1 for provenance of human post-mortem brain samples. All samples were from the frontal cortex of individuals with clinically and pathologically confirmed sporadic Alzheimer's disease (Braak stage 6) or DLB. Non-demented control brain samples were from age-matched individuals who showed no clinical signs of dementia or pathological signs of AD/tauopathy (Braak stage 0). Causes of death for the control individuals, where noted, would not be predicted to impact tau levels/species detected post-mortem.

28.2 Western blot: See Example 27 for details.

Commercial antibodies used were: HT7 (targeting amino acids 159-163; Invitrogen, Carlsbad, CA, USA), Tau5 (targeting amino acids 210-241; Thermo Fisher Scientific, Waltham, MA, USA), Tau13 (targeting amino acids 2-18; Abcam, Cambridge, UK), or Tau46 (targeting amino acids 404-441; New England Biolabs, Ipswich, MA, USA).

### Example 29. Affinity Maturation of Humanised Antibody Clone #44 VH4VK4

Humanised antibody clone #44_VH4VK4 was affinity matured using a conventional mutagenesis approach with degenerate oligos (NNK) to mutate residues in VH CDR3 (Library 1), VL CDR3 (Libraries 2A and 2B) and VH CDR2 (Library 3). Libraries were expressed as scFv and multiple rounds of both soluble and passive selections completed to identify the highest affinity scFv. Library 2A did not yield clones worthy of further study. None of the passive selections showed any binding in polyclonal phage ELISA. A total of 2486 individual clones were screened in phage ELISAs: 945 from Library 1, 222 from Library 2B, and 1319 from Library 3. 88 clones were then cherry picked for final binding ELISA: 64 from Library 3, 1 from Library 2B and 23 from Library 1. This plate comprised 20 unique VH sequences (15 from library 3, 5 from Library 1).

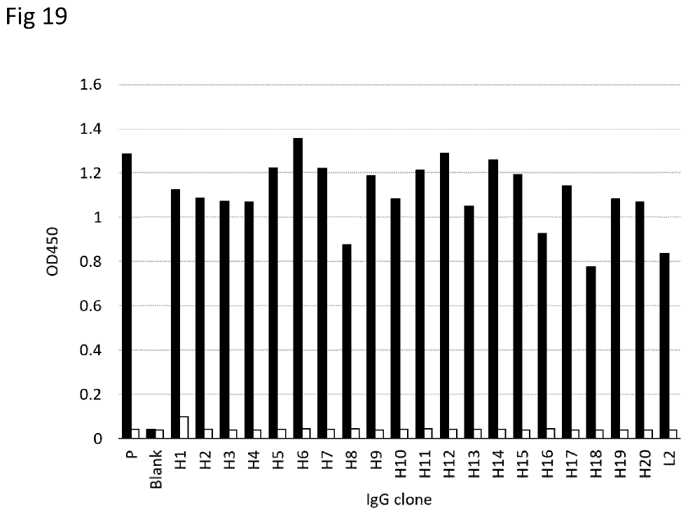

The twenty new VH (SEQ ID NOS: 183 to 206) and one new VK sequence (SEQ ID NO: 207) were prioritised (sequence alignments are provided in FIG. 18) and expressed in combination with the parental VH (pVH) or VK (pVL) as appropriate to create 21 new variants. When expressed in mammalian cells, all new IgG variants form antibodies that bind to full length recombinant 2N4R tau, as demonstrated by ELISA (FIG. 19). Background ELISA signal is low, demonstrating that antibody-related signal is due to a specific interaction with tau. All supernatants were concentration matched and tested at 30 ng/ml.

Biacore single cycle kinetics (SCK) experiments enable calculation of antibody K_{D}. For new variants, K_{D}s range from 593 pM to 34.2 nM (for H04/pVL and H18/pVL respectively; summarised in Table 15). Additional variants combining H06, H07, H09, H13 or H14 with novel VL variant, L2 were tested. None performed better than the same VH combined with pVL, suggesting that VL CDR3 is not a major determinant of antibody binding to tau.

Affinity maturation resulted in 4 novel antibodies with higher affinity to tau than the parental humanised clone #44 VH4VK4 (SEQ ID NO: 154/SEQ ID NO: 160): H01/pVL (SEQ ID NO: 183/SEQ ID NO: 160), H02/pVL (SEQ ID NO: 184/SEQ ID NO: 160), H04/pVL (SEQ ID NO: 186/SEQ ID NO: 160), H06/pVL (SEQ ID NO: 188/SEQ ID NO: 160). These clones all contain mutations in VH CDR3, with H06 also including VH CDR2 mutations, implicating VH CDR2 and VH CDR3 as key regions for #44_VH4VK4-related antibody binding to tau. Mutation of VL CDR3 (Libraries 2A and 2B) did not yield improvements in antibody affinity.

Recombination of H06 (VH CDR2) with H01, H02 or H04 (VH CDR3) to form H06-01/pVL, H06-02/pVL, H06-04/pVL did not result in increases in affinity compared to the original H06/pVL clone (see Table 16). Additional recombination of H16 CDR2 with H04 CDR3 also yielded no increases in affinity compared to the original clones.

Data demonstrated the importance of VH CDR3 and VH CDR2 in determining the affinity of #44_VH4VK4-related antibody interactions with tau. Specifically, as shown in Table 14:

TABLE 14

| | Parental antibody amino | Variant amino acid |
|---|---|---|
| | acid residue pVH | residue in optimized clones |
| VH CDR2 amino | | |
| acid residue | | |
| 51 | C | V or A |
| 54 | R | A or S |
| 55 | R | A or V |
| 57 | G | H, N, R, A or S |
| VH CDR3 amino | | |
| acid residue | | |
| 96 | S | V, R or A |
| 98 | A | S, D, H or T |
| 102 | P | V or Y |

Abzena's proprietary iTope analysis was run to assess the presence of predicted MHC class II binding peptides, with a view to minimising the presence of high affinity binding sites in prioritised leads. A summary of this analysis is provided in Table 16. Novel VH clones contain 1-4 predicted high affinity sites (compared to 1 for the parental #44_VH4) and 2-5 moderate affinity sites (compared to 4 for the parental #44_VH4). The single novel VK clone shares the 2 moderate affinity and 2 high affinity sites of the parental #44_VK4 clone.

29.1 Affinity maturation by phage display-Library design and construction: Libraries were designed as outlined above to mutate regions in VH CDR3 (region from D95 to P102, Library 1); VH CDR2 (residues from C50 to F58, excluding 151, G55 and T57 that were kept constant, Library 3), VL CDR3 (L89 to S93, N96 and V97, Library 2A; the four residues between the two Cys in positions C94 and C95D, keeping both Cys constant; Library 2B). Libraries were constructed using standard methods. Oligos were designed to introduce randomisations in the targeted sequences with NcoI or NotI restriction sites. Purified, amplified DNA for all three libraries was digested using NcoI and NotI and ligated into the similarly cut phagemid vector (pANT65). Ligated DNA was cleaned and eluted in nuclease-free water using a Roche HP PCR Product Purification Kit (Roche Life Sciences, Burgess Hill, UK) and transformed by electroporation into freshly prepared electrocompetent TG1 cells. The following day, colonies were counted, plates scraped, and glycerol stocks prepared. Libraries were electroporated multiple times in order to sufficiently cover the theoretical library diversity. For each of the libraries a coverage of ten-fold or greater was obtained. This gives a statistical probability of covering 99% of the whole library.

29.2 Affinity maturation—Library phage rescue: Bacteria from the four libraries were inoculated into 150 mL 2TYCG (2%) cultures using inocula at least 100× the observed library diversity. Cultures were grown to mid-log phase (OD₆₀₀ₙₘ>0.4) and the total number of cells estimated (based on an OD600 nm of 1=5×10⁸ cells/mL). Helper phage (M13K07) (New England BioLabs, Hitchin, UK) were added at a multiplicity of infection of 10 and incubated for 1 hour at 150 rpm, then centrifuged, resuspended in 2TYCK media and grown overnight at 30° C. The following day, phage were harvested by recovering the culture supernatant by centrifugation followed by precipitation using 3/10th volume of chilled 20% PEG/2.5 M NaCl. After 1-hour incubation on ice, precipitated phage were recovered by centrifugation and the pellet resuspended in 1×PBS pH 7.4. The supernatant was re-centrifuged to remove any cellular debris, following which the supernatant was re-precipitated as described above. The precipitated phage were resuspended in 1×PBS pH 7.4.

29.3 Affinity improved phage selections: For the in solution selections, based on affinity improvement, each of the libraries were pre-blocked with MPBS (containing 3% milk) then phage were incubated with decreasing concentrations of soluble biotinylated full-length Tau (50 nM) for 1 hour at room temperature. Pre-blocked streptavidin paramagnetic beads were added to each selection and rotated for 5 min. Streptavidin-antigen-phage complexes were washed with 8 washing steps with PBST followed by a PBS wash, using a KingFisher 1 mL (ThermoFisher, Waltham, USA). Phage were eluted from the beads by the addition of 50 mM HCl then neutralised by the addition of 1 M Tris-HCl pH 9.0. Phage were then infected into logarithmic phase TG1 cells and plated out on LB Agar plates containing carbenicillin. For all selections, a “no antigen” control selection was performed following the protocol as described above but omitting the antigen. Comparison of the numbers of colonies on output plates from selections either with, or without, antigen provides information as to the success of any particular selection.

For the passive selections each of the libraries were pre-blocked with MPBS (containing 5% milk) then phage were incubated with immune tube coated with decreasing concentrations of non-biotinylated full-length human Tau (22.2 nM) and blocked with MPBS for 1 hour at room temperature. Antigen-phage complexes were washed with ×3 with PBST followed by a PBS wash. Phage were eluted from the beads by the addition of 50 mM HCl then neutralised by the addition of 1 M Tris-HCl pH 9.0. Phage were then infected into logarithmic phase TG1 cells as described for in solution selections.

29.4 Expression and testing of scFv: Soluble scFv were initially expressed and tested for binding to Tau, as protein secreted into the media after IPTG induction, in a single point binding ELISA format. Individual colonies from selected selection outputs were picked into 1 mL 2TYCG (0.1%) media and grown by shaking at 37° C. for 5 h. Cultures were induced by adding IPTG to a final concentration of 1 mM and then grown overnight, with shaking, at 30° C. The following day, cultures were centrifuged and the supernatant tested by ELISA (1:20 dilution). Colonies from scFv, expressed into the media, for all the libraries were screened with the parental scFv and an irrelevant scFv included on each assay plate as controls.

Nunc Immuno MaxiSorp 96 well flat bottom microtitre plates were coated with 50 UL Tau at 1.0 μg/mL overnight at 4° C. Plates were washed and blocked for 1 h at room temperature with 5% MPBS. Induced media containing expressed scFvs was diluted 1:20, added to the blocked plate (100 μL per well), and incubated on the plate for 1 h at room temperature. Plates were then washed, the binding of the scFvs to the target was detected with an anti-HIS antibody-HRP (Sigma-Aldrich, Dorset, UK) and developed with TMB substrate (Invitrogen, Loughborough, UK). The reaction was stopped with 1 M HCl, absorbance read at 450 nm on a Dynex Technologies MRX TC II plate reader and the binding data plotted.

29.5 CD4+ T cell epitope avoidance (analysis by iTope™): See Example 21.2.

29.6 Generation of novel antibody variants: See Example 21.3

29.7 Assessment of antibody binding to tau (ELISA): See Example 21.4. Here supernatants were concentration matched to 30 ng/ml (using Octet-based estimations of titre, calculated by Abzena using standard methods).

29.8 Assessment of antibody binding to tau (Biacore SCK analysis): See Example 21.5.

TABLE 15
Sequences of CDRs from the affinity matured antibody panel

| Rank | | HC sequence | | | LC sequence | | |
|---|---|---|---|---|---|---|---|
| order | Code | CDR1 | CDR2 | CDR3 | CDR1 | CDR2 | CDR3 |
| | #44 (parental | SYAMA | CIDRRGGTFYASWVKG | DSGAFDP | QASQSVYDNYLA | AASNLAS | LGEFSCTTTDCNV |
| | humanised) | (SEQ ID NO: 20) | (SEQ ID NO: 21) | (SEQ ID NO: 22) | (SEQ ID NO: 23) | (SEQ ID NO: 24) | (SEQ ID NO: 25) |
| 4 | #44_VH4_H01 | SYAMA | CIDRRGGTFYASWVKG | DVGSFDV | QASQSVYDNYLA | AASNLAS | LGEFSCTTTDCNV |
| | | (SEQ ID NO: 20) | (SEQ ID NO: 21) | (SEQ ID NO: 161) | (SEQ ID NO: 23) | (SEQ ID NO: 24) | (SEQ ID NO: 25) |
| 2 | #44_VH4_H02 | SYAMA | CIDRRGGTFYASWVKG | DRGDFDV | QASQSVYDNYLA | AASNLAS | LGEFSCTTTDCNV |
| | | (SEQ ID NO: 20) | (SEQ ID NO: 21) | (SEQ ID NO: 162) | (SEQ ID NO: 23) | (SEQ ID NO: 24) | (SEQ ID NO: 25) |
| | #44_VH4_H03 | SYAMA | CIDRRGGTFYASWVKG | DAGAFDV | QASQSVYDNYLA | AASNLAS | LGEFSCTTTDCNV |
| | | (SEQ ID NO: 20) | (SEQ ID NO: 21) | (SEQ ID NO: 163) | (SEQ ID NO: 23) | (SEQ ID NO: 24) | (SEQ ID NO: 25) |
| 1 | #44_VH4_H04 | SYAMA | CIDRRGGTFYASWVKG | DAGSFHP | QASQSVYDNYLA | AASNLAS | LGEFSCTTTDCNV |
| | | (SEQ ID NO: 20) | (SEQ ID NO: 21) | (SEQ ID NO: 164) | (SEQ ID NO: 23) | (SEQ ID NO: 24) | (SEQ ID NO: 25) |
| | #44_VH4_H05 | SYAMA | CIDRRGGTFYASWVKG | DAGHFDY | QASQSVYDNYLA | AASNLAS | LGEFSCTTTDCNV |
| | | (SEQ ID NO: 20) | (SEQ ID NO: 21) | (SEQ ID NO: 165) | (SEQ ID NO: 23) | (SEQ ID NO: 24) | (SEQ ID NO: 25) |
| 3 | #44_VH4_H06 | SYAMA | VIDAAGHTFYASWVKG | DSGTFDP | QASQSVYDNYLA | AASNLAS | LGEFSCTTTDCNV |
| | | (SEQ ID NO: 20) | (SEQ ID NO: 167) | (SEQ ID NO: 166) | (SEQ ID NO: 23) | (SEQ ID NO: 24) | (SEQ ID NO: 25) |
| | #44_VH4_H07 | SYAMA | AIDAAGNTFYASWVKG | DSGAFDP | QASQSVYDNYLA | AASNLAS | LGEFSCTTTDCNV |
| | | (SEQ ID NO: 20) | (SEQ ID NO: 168) | (SEQ ID NO: 22) | (SEQ ID NO: 23) | (SEQ ID NO: 24) | (SEQ ID NO: 25) |
| | #44_VH4_H08 | SYAMA | AIDAAGRTFYASWVKG | DSGAFDP | QASQSVYDNYLA | AASNLAS | LGEFSCTTTDCNV |
| | | (SEQ ID NO: 20) | (SEQ ID NO: 169) | (SEQ ID NO: 22) | (SEQ ID NO: 23) | (SEQ ID NO: 24) | (SEQ ID NO: 25) |
| | #44_VH4_H09 | SYAMA | AIDARGNTFYASWVKG | DSGAFDP | QASQSVYDNYLA | AASNLAS | LGEFSCTTTDCNV |
| | | (SEQ ID NO: 20) | (SEQ ID NO: 170) | (SEQ ID NO: 22) | (SEQ ID NO: 23) | (SEQ ID NO: 24) | (SEQ ID NO: 25) |
| | #44_VH4_H10 | SYAMA | AIDARGRTFYASWVKG | DSGAFDP | QASQSVYDNYLA | AASNLAS | LGEFSCTTTDCNV |
| | | (SEQ ID NO: 20) | (SEQ ID NO: 171) | (SEQ ID NO: 22) | (SEQ ID NO: 23) | (SEQ ID NO: 24) | (SEQ ID NO: 25) |
| | #44_VH4_H11 | SYAMA | AIDARGATFYASWVKG | DSGAFDP | QASQSVYDNYLA | AASNLAS | LGEFSCTTTDCNV |
| | | (SEQ ID NO: 20) | (SEQ ID NO: 172) | (SEQ ID NO: 22) | (SEQ ID NO: 23) | (SEQ ID NO: 24) | (SEQ ID NO: 25) |
| | #44_VH4_H12 | SYAMA | AIDARGSTFYASWVKG | DSGAFDP | QASQSVYDNYLA | AASNLAS | LGEFSCTTTDCNV |
| | | (SEQ ID NO: 20) | (SEQ ID NO: 173) | (SEQ ID NO: 22) | (SEQ ID NO: 23) | (SEQ ID NO: 24) | (SEQ ID NO: 25) |
| | #44_VH4_H13 | SYAMA | AIDSAGNTFYASWVKG | DSGAFDP | QASQSVYDNYLA | AASNLAS | LGEFSCTTTDCNV |
| | | (SEQ ID NO: 20) | (SEQ ID NO: 174) | (SEQ ID NO: 22) | (SEQ ID NO: 23) | (SEQ ID NO: 24) | (SEQ ID NO: 25) |
| | #44_VH4_H14 | SYAMA | AIDSAGSTFYASWVKG | DSGAFDP | QASQSVYDNYLA | AASNLAS | LGEFSCTTTDCNV |
| | | (SEQ ID NO: 20) | (SEQ ID NO: 175) | (SEQ ID NO: 22) | (SEQ ID NO: 23) | (SEQ ID NO: 24) | (SEQ ID NO: 25) |
| | #44_VH4_H15 | SYAMA | AIDSVGNTFYASWVKG | DSGAFDP | QASQSVYDNYLA | AASNLAS | LGEFSCTTTDCNV |
| | | (SEQ ID NO: 20) | (SEQ ID NO: 176) | (SEQ ID NO: 22) | (SEQ ID NO: 23) | (SEQ ID NO: 24) | (SEQ ID NO: 25) |
| | #44_VH4_H16 | SYAMA | AIDRAGNTFYASWVKG | DSGAFDP | QASQSVYDNYLA | AASNLAS | LGEFSCTTTDCNV |
| | | (SEQ ID NO: 20) | (SEQ ID NO: 177) | (SEQ ID NO: 22) | (SEQ ID NO: 23) | (SEQ ID NO: 24) | (SEQ ID NO: 25) |
| | #44_VH4_H17 | SYAMA | AIDRVGNTFYASWVKG | DSGAFDP | QASQSVYDNYLA | AASNLAS | LGEFSCTTTDCNV |
| | | (SEQ ID NO: 20) | (SEQ ID NO: 178) | (SEQ ID NO: 22) | (SEQ ID NO: 23) | (SEQ ID NO: 24) | (SEQ ID NO: 25) |
| | #44_VH4_H18 | SYAMA | AIDAGGNTFYASWVKG | DSGAFDP | QASQSVYDNYLA | AASNLAS | LGEFSCTTTDCNV |
| | | (SEQ ID NO: 20) | (SEQ ID NO: 179) | (SEQ ID NO: 22) | (SEQ ID NO: 23) | (SEQ ID NO: 24) | (SEQ ID NO: 25) |
| | #44_VH4_H19 | SYAMA | AIDSRGATFYASWVKG | DSGAFDP | QASQSVYDNYLA | AASNLAS | LGEFSCTTTDCNV |
| | | (SEQ ID NO: 20) | (SEQ ID NO: 180) | (SEQ ID NO: 22) | (SEQ ID NO: 23) | (SEQ ID NO: 24) | (SEQ ID NO: 25) |
| | #44_VH4_H20 | SYAMA | AIDSRGSTFYASWVKG | DSGAFDP | QASQSVYDNYLA | AASNLAS | LGEFSCTTTDCNV |
| | | (SEQ ID NO: 20) | (SEQ ID NO: 181) | (SEQ ID NO: 22) | (SEQ ID NO: 23) | (SEQ ID NO: 24) | (SEQ ID NO: 25) |
| | #44_VH4_H06-H01 | SYAMA | VIDAAGHTFYASWVKG | DVGSFDV | QASQSVYDNYLA | AASNLAS | LGEFSCTTTDCNV |
| | | (SEQ ID NO: 20) | (SEQ ID NO: 167) | (SEQ ID NO: 161) | (SEQ ID NO: 23) | (SEQ ID NO: 24) | (SEQ ID NO: 25) |
| | #44_VH4_H06-H02 | SYAMA | VIDAAGHTFYASWVKG | DRGDFDV | QASQSVYDNYLA | AASNLAS | LGEFSCTTTDCNV |
| | | (SEQ ID NO: 20) | (SEQ ID NO: 167) | (SEQ ID NO: 162) | (SEQ ID NO: 23) | (SEQ ID NO: 24) | (SEQ ID NO: 25) |
| 5 | #44_VH4_H06-H04 | SYAMA | VIDAAGHTFYASWVKG | DAGSFHP | QASQSVYDNYLA | AASNLAS | LGEFSCTTTDCNV |
| | | (SEQ ID NO: 20) | (SEQ ID NO: 167) | (SEQ ID NO: 164) | (SEQ ID NO: 23) | (SEQ ID NO: 24) | (SEQ ID NO: 25) |
| | #44_VH4_H16-H04 | SYAMA | AIDRAGNTFYASWVKG | DAGSFHP | QASQSVYDNYLA | AASNLAS | LGEFSCTTTDCNV |
| | | (SEQ ID NO: 20) | (SEQ ID NO: 177) | (SEQ ID NO: 164) | (SEQ ID NO: 23) | (SEQ ID NO: 24) | (SEQ ID NO: 25) |
| | #44_VK4_L2 | SYAMA | CIDRRGGTFYASWVKG | DSGAFDP | QASQSVYDNYLA | AASNLAS | LGEFSCQDTDCNV |
| | | (SEQ ID NO: 20) | (SEQ ID NO: 21) | (SEQ ID NO: 22) | (SEQ ID NO: 23) | (SEQ ID NO: 24) | (SEQ ID NO: 182) |
| | #44_VH4_H06/L2 | SYAMA | VIDAAGHTFYASWVKG | DSGTFDP | QASQSVYDNYLA | AASNLAS | LGEFSCQDTDCNV |
| | | (SEQ ID NO: 20) | (SEQ ID NO: 167) | (SEQ ID NO: 166) | (SEQ ID NO: 23) | (SEQ ID NO: 24) | (SEQ ID NO: 182) |
| | #44_VH4_H07/L2 | SYAMA | AIDAAGNTFYASWVKG | DSGAFDP | QASQSVYDNYLA | AASNLAS | LGEFSCQDTDCNV |
| | | (SEQ ID NO: 20) | (SEQ ID NO: 168) | (SEQ ID NO: 22) | (SEQ ID NO: 23) | (SEQ ID NO: 24) | (SEQ ID NO: 182) |
| | #44_VH4_H09/L2 | SYAMA | AIDARGNTFYASWVKG | DSGAFDP | QASQSVYDNYLA | AASNLAS | LGEFSCQDTDCNV |
| | | (SEQ ID NO: 20) | (SEQ ID NO: 170) | (SEQ ID NO: 22) | (SEQ ID NO: 23) | (SEQ ID NO: 24) | (SEQ ID NO: 182) |
| | #44_VH4_H13/L2 | SYAMA | AIDSAGNTFYASWVKG | DSGAFDP | QASQSVYDNYLA | AASNLAS | LGEFSCQDTDCNV |
| | | (SEQ ID NO: 20) | (SEQ ID NO: 174) | (SEQ ID NO: 22) | (SEQ ID NO: 23) | (SEQ ID NO: 24) | (SEQ ID NO: 182) |
| | #44_VH4_H14/L2 | SYAMA | AIDSAGSTFYASWVKG | DSGAFDP | QASQSVYDNYLA | AASNLAS | LGEFSCQDTDCNV |
| | | (SEQ ID NO: 20) | (SEQ ID NO: 175) | (SEQ ID NO: 22) | (SEQ ID NO: 23) | (SEQ ID NO: 24) | (SEQ ID NO: 182) |

TABLE 16
Summary of iTope analysis output showing the number of predicted
moderate and high affinity MHC class II binding peptides present
in the parental clone #44_VH4VK4 (pVH, pVL) and affinity
matured variants of VH (H01-H20) and VK (L02).

MHCII binding peptides

| | Moderate | High |
|---|---|---|
| Antibody clone | affinity | affinity |
| pVH | 4 | 1 |
| pVL | 2 | 2 |
| H01 | 5 | 2 |
| H02 | 5 | 1 |
| H03 | 5 | 1 |
| H04 | 4 | 1 |
| H05 | 4 | 1 |
| H06 | 2 | 3 |
| H07 | 3 | 1 |
| H08 | 3 | 1 |
| H09 | 3 | 1 |
| H10 | 3 | 1 |
| H11 | 4 | 1 |
| H12 | 3 | 2 |
| H13 | 3 | 2 |
| H14 | 3 | 4 |
| H15 | 2 | 2 |
| H16 | 2 | 1 |
| H17 | 2 | 1 |
| H18 | 3 | 1 |
| H19 | 3 | 2 |
| H20 | 2 | 3 |
| L02 | 2 | 2 |

TABLE 17
Summary of binding analysis (SCK) data for affinity matured variants
of clone #44_VH4VK4 binding to full length recombinant 2N4R tau.
The parental clone #44_VH4VK4 is shown as pVH/pVL (VH SEQ
ID NO: 154 and VL SEQ ID NO: 160), and ‘Fold improvement’ values
are calculated relative to this clone. All clones are expressed as
hIgG1. HEK titres are based on Octet analysis at Day 7 post-transfection.

| | ka | kd | K_{D} | Rmax | Chi² | Fold | Conc. |
|---|---|---|---|---|---|---|---|
| Ligand | (1/Ms) | (1/s) | (M) | (RU) | (RU²) | change | (ug/ml) |
| pVH/pVL | 1.82E+06 | 1.20E−02 | 6.58E−09 | 24.4 | 0.0683 | 1.00 | 3.4 |
| H01/pVL | 2.97E+06 | 8.05E−03 | 2.71E−09 | 19.8 | 0.078 | 2.43 | 2.61 |
| H02/pVL | 2.78E+06 | 3.24E−03 | 1.17E−09 | 21.4 | 0.0421 | 5.62 | 2.45 |
| H03/pVL | 2.36E+06 | 1.51E−02 | 6.40E−09 | 24 | 0.131 | 1.03 | 3.27 |
| H04/pVL | 2.86E+06 | 1.70E−03 | 5.93E−10 | 22.9 | 0.0964 | 11.10 | 1.5 |
| H05/pVL | 4.18E+06 | 2.03E−02 | 4.84E−09 | 25.4 | 0.118 | 1.36 | 8.26 |
| H06/pVL | 3.90E+06 | 4.44E−03 | 1.14E−09 | 25.9 | 0.0312 | 5.77 | 2.91 |
| H07/pVL | 3.91E+09 | 2.87E+01 | 7.35E−09 | 23.4 | 0.605 | 0.90 | 6.87 |
| H08/pVL | 4.26E+05 | 1.10E−02 | 2.57E−08 | 41 | 0.301 | 0.26 | 3.57 |
| H09/pVL | 3.25E+06 | 3.20E−02 | 9.85E−09 | 26.6 | 0.515 | 0.67 | 6.47 |
| H10/pVL | 1.98E+09 | 1.97E+01 | 9.95E−09 | 26.6 | 0.168 | 0.66 | 4.29 |
| H11/pVL | 3.86E+09 | 2.18E+01 | 5.65E−09 | 27.5 | 0.328 | 1.16 | 3.94 |
| H12/pVL | 4.16E+07 | 2.51E−01 | 6.04E−09 | 29.9 | 0.354 | 1.09 | 4.73 |
| H13/pVL | 8.55E+05 | 1.48E−02 | 1.73E−08 | 34.8 | 0.371 | 0.38 | 2.98 |
| H14/pVL | 4.51E+09 | 2.87E+01 | 6.37E−09 | 25.4 | 0.443 | 1.03 | 3.77 |
| H15/pVL | 4.12E+06 | 3.61E−02 | 8.76E−09 | 26.8 | 0.518 | 0.75 | 4.88 |
| H16/pVL | 2.38E+09 | 2.27E+01 | 9.51E−09 | 18.4 | 0.307 | 0.69 | 4.95 |
| H17/pVL | 4.81E+07 | 3.06E−01 | 6.36E−09 | 28 | 0.27 | 1.03 | 3.3 |
| H18/pVL | 2.87E+05 | 9.83E−03 | 3.42E−08 | 50.2 | 0.308 | 0.19 | 2.77 |
| H19/pVL | 1.26E+06 | 1.22E−02 | 9.72E−09 | 30.6 | 0.427 | 0.68 | 2.54 |
| H20/pVL | 4.60E+06 | 4.04E−02 | 8.78E−09 | 28.7 | 0.434 | 0.75 | 2.16 |
| pVH/L2 | 3.03E+09 | 2.23E+01 | 7.38E−09 | 16.9 | 0.154 | 0.89 | 4.91 |
| H06/L2 | 4.52E+06 | 8.41E−03 | 1.86E−09 | 28 | 0.102 | 3.54 | — |
| H07/L2 | 4.97E+05 | 1.24E−02 | 2.49E−08 | 41.6 | 0.377 | 0.26 | — |
| H09/L2 | 7.28E+05 | 1.06E−02 | 1.46E−08 | 32.6 | 0.394 | 0.45 | — |
| H13/L2 | 3.58E+05 | 8.44E−03 | 2.35E−08 | 42 | 0.383 | 0.28 | — |
| H14/L2 | 1.28E+06 | 1.61E−02 | 1.26E−08 | 27.3 | 0.32 | 0.52 | — |
| H06-H01/pVL | 3.15E+06 | 1.21E−02 | 3.85E−09 | 54 | 0.719 | 1.71 | 7.59 |
| H06-H02/pVL | 5.75E+06 | 4.89E−03 | 8.51E−10 | 47.5 | 0.142 | 7.73 | 6.59 |
| H06-H04/pVL | 6.14E+06 | 4.43E−03 | 7.22E−10 | 51.1 | 0.0826 | 9.11 | 7.81 |
| H16-H04/pVL | 4.99E+06 | 4.17E−03 | 8.35E−10 | 54.8 | 0.235 | 7.88 | 7.43 |

### Example 30. Affinity Matured IgG Bind to Tau with High Affinity

The top four affinity matured humanised variants were selected for larger scale expression as hIgG1 and further characterisation (based on data summarised in Table 17): #44_VH4VK4_H01/pVL (SEQ ID NO: 183 and SEQ ID NO: 160); #44_VH4VK4_H02/pVL (SEQ ID NO: 184 and SEQ ID NO: 160); #44_VH4VK4_H04/pVL (SEQ ID NO: 186 and SEQ ID NO: 160); and #44_VH4VK4_H06/pVL (SEQ ID NO: 188 and SEQ ID NO: 160). CHO cells were used for this larger scale production to maximise yields. When referenced as _H01, _H02 etc, it should be understood that these clones derive from #44_VH4VK4 and include the parental VL (pVL). All clones were expressed as human IgG1 (hIgG1). It is expected that binding characteristics would be unchanged if clones were to be expressed as different isotypes/formats.

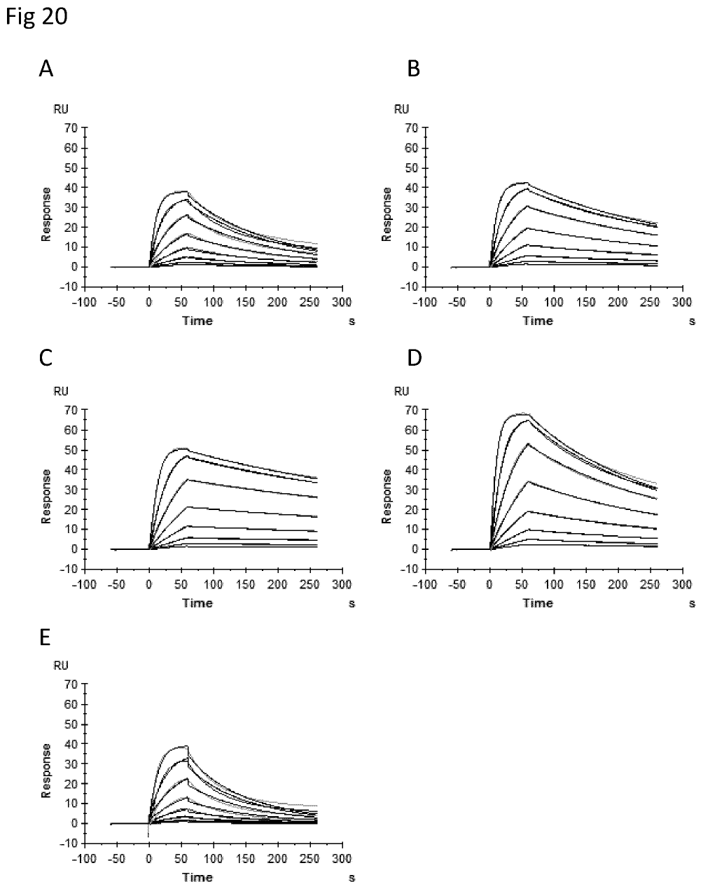

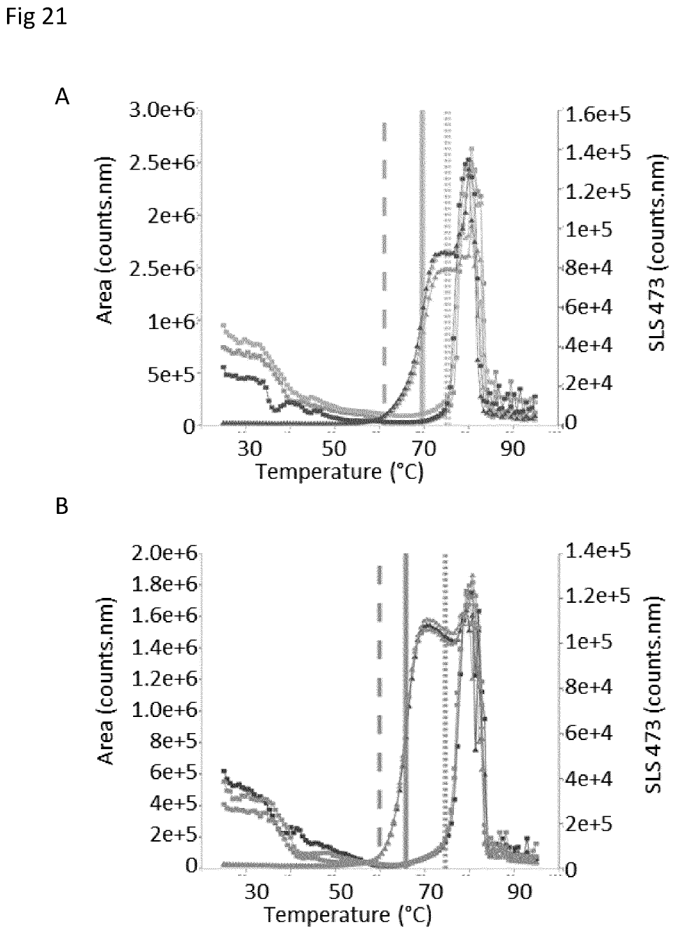

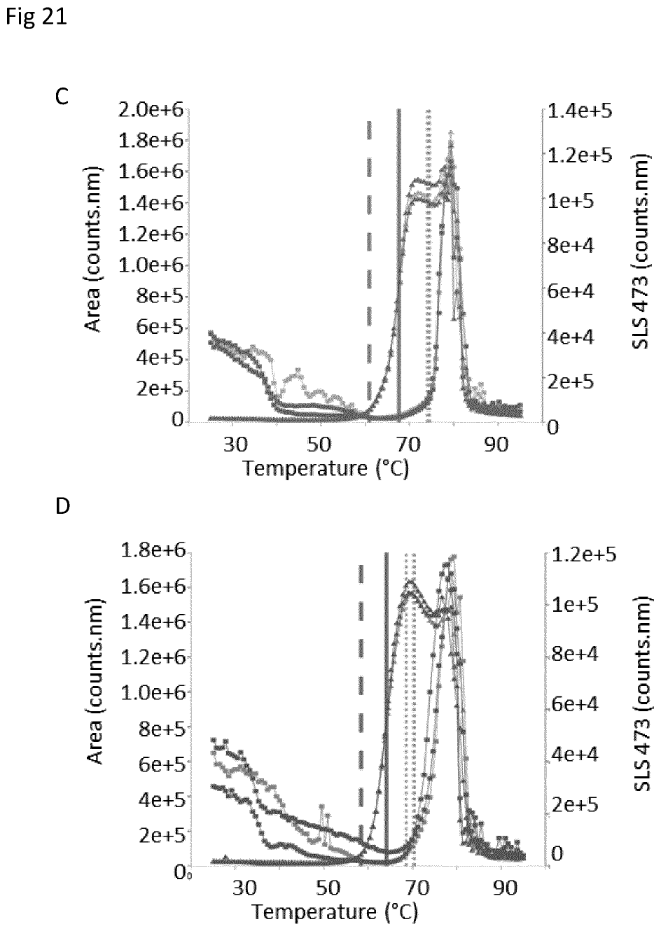

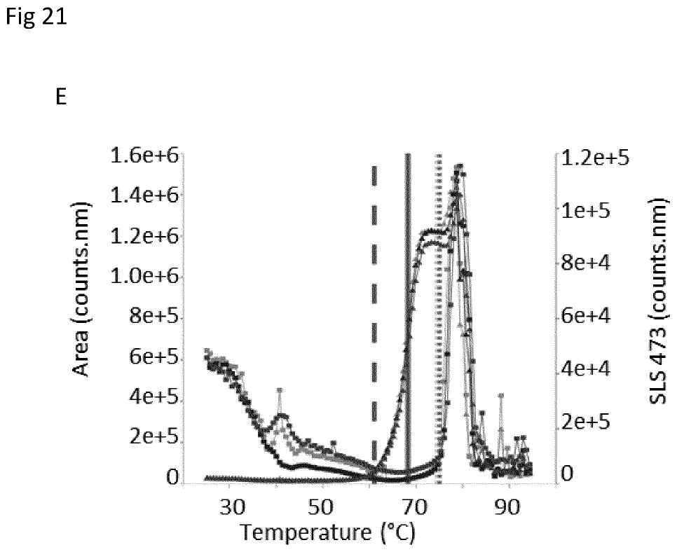

Biacore multi-cycle kinetics (MCK) analyses for antibody binding to recombinant 2N4R tau are summarised in FIG. 20 and Table 18. All four variants demonstrate higher affinity for tau than the parental #44_VH4VK4: 0.736 nM (_H04), 1.52 nM (_H02), 1.53 nM (_H06), and 4.01 nM (_H01), compared to 7.76 nM for the parental IgG tested in parallel. Slight variations in R_{MAX} are considered likely to be due to differences in ligand capture and not reflective of significant variation in antibody binding characteristics.

As described in Example 21, it is predicted that activity described for human tau, will be applicable to other mammalian species of tau, if the target sequence (SEQ ID NO: 1) is present and accessible.

30.1 Production of Protein A purified hIgG1 antibodies: See Example 22.1.

30.2 Assessment of antibody binding to tau (Biacore MCK analysis): See Example 22.2. Note that in this experiment, flow rate was increased to 100 μL/min to minimise the impact of mass transport limitations.

TABLE 18
Summary of Biacore MCK analysis for purified affinity matured variants of clone
#44_VH4VK4 (H01-H06; hIgG1) binding to full length recombinant 2N4R tau. The parental
clone #44_VH4VK4 is shown as pVH/pVL and ‘relative KD’ values are
calculated relative to this clone. New variants were tested as SEC purified samples.

| | | | | Fold | Rₘₐₓ | Chi² |
|---|---|---|---|---|---|---|
| Ligand | kₐ (1/Ms) | k_{d} (1/s) | K_{D} (M) | improvement | (RU) | (RU²) |
| pVH/pVL | 1.89E+06 | 1.47E−02 | 7.76E−09 | 1.0 | 17.5 | 0.0511 |
| (SEQ ID NO: 154 and SEQ | | | | | | |
| ID NO: 160) | | | | | | |
| H01 | 2.39E+06 | 9.57E−03 | 4.01E−09 | 1.9 | 20.1 | 0.0456 |
| (SEQ ID NO: 183 and SEQ | | | | | | |
| ID NO: 160) | | | | | | |
| H02 | 2.43E+06 | 3.70E−03 | 1.52E−09 | 5.1 | 21 | 0.0318 |
| (SEQ ID NO: 184 and SEQ | | | | | | |
| ID NO: 160) | | | | | | |
| H04 | 2.51E+06 | 1.85E−03 | 7.36E−10* | 10.5 | 25.2 | 0.0311 |
| (SEQ ID NO: 186 and SEQ | | | | | | |
| ID NO: 160) | | | | | | |
| H06 | 3.43E+06 | 5.24E−03 | 1.53E−09* | 5.0 | 34.8 | 0.0857 |
| (SEQ ID NO: 188 and SEQ | | | | | | |
| ID NO: 160) | | | | | | |

*On the border of mass transport limitation.

### Example 31. Affinity Matured IgG are Thermodynamically Stable

To assess the potential of affinity matured humanised variants for development into therapeutic antibodies, the thermal stability of each was assessed. Data are summarised in Table 19 and FIG. 36. Average melting temperature (Tm) ranged from 64.2° C. for #44_VH4VK4_H06/pVL to 69.6° C. for #44_VH4VK4_H01/pVL, which is considered acceptable for a therapeutic antibody.

TABLE 19
Summary of thermal stability profiling data for
novel variants of clone #44_VH4VK4 (H01-
H06). Parental clone #44 VH4VK4 is shown as
pVH/pVL. All antibodies were expressed as hIgG1.

| | Unfolding | | Aggregation |
|---|---|---|---|
| | Average Tₒₙₛₑₜ | Average Tₘ₁ | Average T_{agg} |
| Variant | (° C.) | (° C.) | 473nm (° C.) |
| pVL/pVL | 61.1 | 68.2 | 74.9 |
| H01 | 61.1 | 69.6 | 75.2 |
| H02 | 59.9 | 65.7 | 74.5 |
| H04 | 60.9 | 67.6 | 74.3 |
| H06 | 58.4 | 64.2 | 69.8 |

31.1 Thermal stability analysis: See Example 23.1.

### Example 32. Affinity Matured Variants of Antibody #44_VH4VK4 Inhibit Uptake of Monomeric and Aggregated Tau into Human Neurons

As described in Example 12 and 24, neuronal uptake of ‘toxic’ forms of extracellular tau is proposed to play an important role in the pathogenic spreading of tau observed in tauopathies such as Alzheimer's disease. Anti-tau rabbit IgG targeting SEQ ID NO: 1, including antibody clone #44 (Clone 2) are able to reduce uptake of tau species containing this epitope by human neurons (Example 12). Antibodies exhibiting this activity would be predicted to limit the neuron-neuron propagation of extracellular tau species that include the target epitope in vivo and therefore to be therapeutically useful.

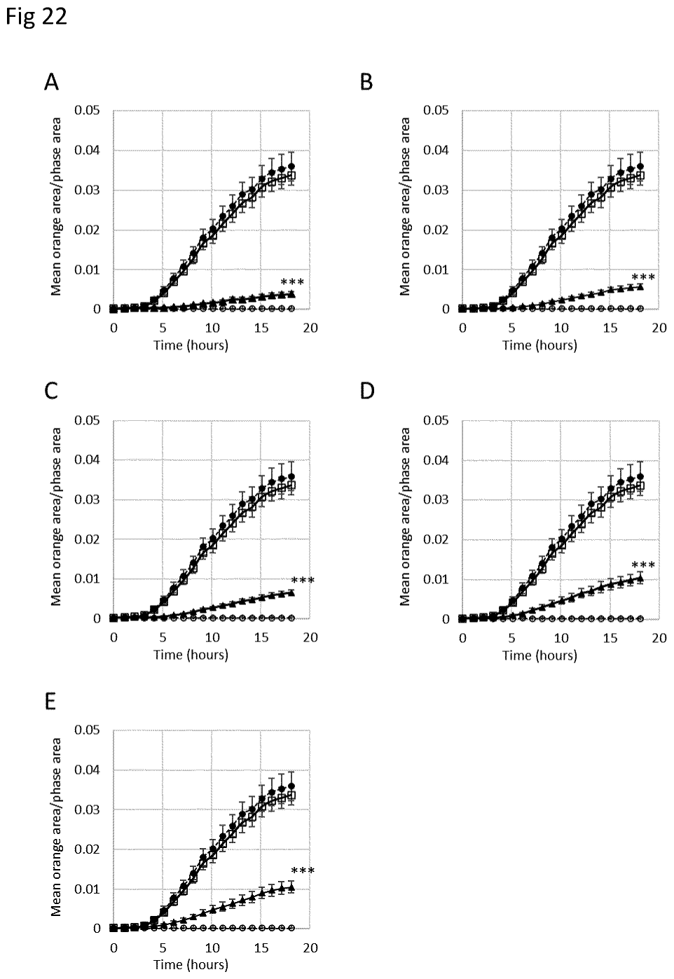

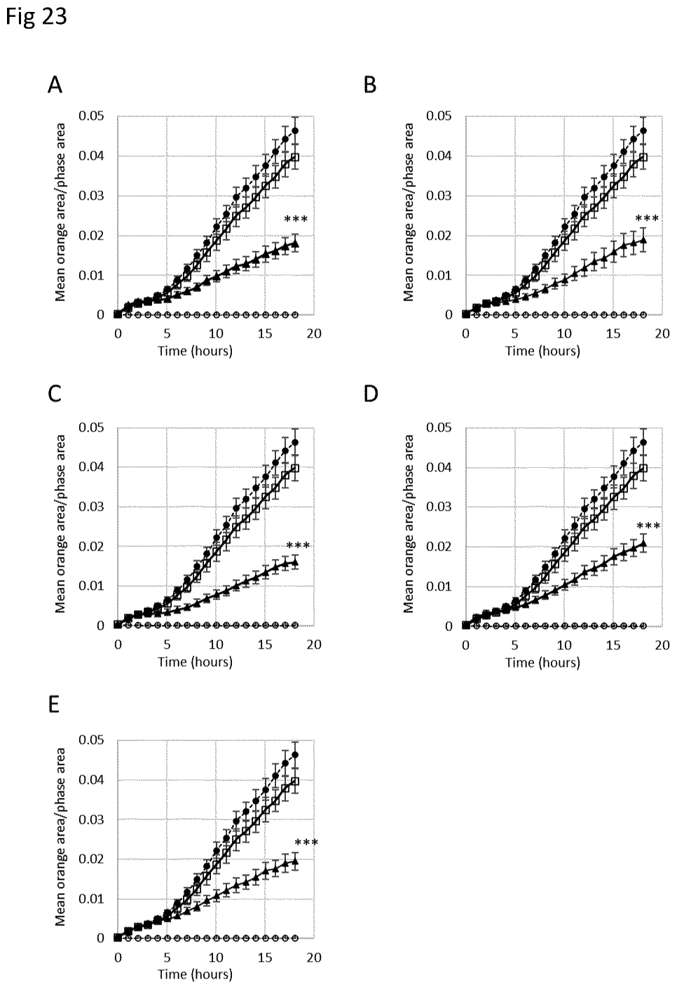

All affinity matured humanised variants of clone #44_VH4VK4 tested (as hIgG1), significantly (P<0.001) inhibited uptake of monomeric tau into human iPSC-derived neurons (FIG. 22) to a similar or greater extent to the parental humanised #44_VH4VK4 clone: by 89.2±1.5% (H01/pVL), 84.1±1.9% (H02/pVL), 81.8±1.7% (VH04/pVL), 71.0±4.1% (H06/pVL), compared to 70.6±4.3% (#44_VH4VK4). All humanised variants tested, also significantly (P<0.001) inhibited uptake of aggregated tau into human iPSC-derived neurons (FIG. 23) to a similar extent to the parental humanised clone, #44_VH4VK4: by 61.1±4.8% (H01/pVL), 59.2±6.4% (H02/pVL), 65.5±3.8% (H04/pVL), 55.0±4.9% (H06/pVL), compared to 58.0±4.7% (#44_VH4VK4). Isotype control human IgG1 antibody had no significant effect on tau uptake in this system (inhibition of 6.2±7.0% and 14.3±6.8% for monomeric and aggregated tau respectively). It should be noted that some variability in absolute levels of tau uptake and absolute level of antibody-mediated inhibition is expected in these complex cell-based models. This variability is likely due to variability in expression of proteins required for tau uptake, in addition to the usual experimental variability. Data shown in this Example are representative of data observed in multiple experimental runs.

Data demonstrated that, like the parental humanised antibody (#44_VH4VK4), affinity matured antibodies targeting the amino acid sequence of SEQ ID NO: 1 were able to reduce the uptake of tau species containing this epitope, by human neurons. Such antibodies would therefore be predicted to limit the neuron-to-neuron propagation of extracellular tau species (monomeric and multimeric, including full length and truncated forms) that include the epitope formed by the amino acid sequence of (SEQ ID NO: 1) in Alzheimer's disease and tauopathies, and thereby reduce/slow the progression of clinical symptoms in patients. Data shown were generated using hIgG1. This activity is CDR-dependent (rather than isotype-dependent) and would therefore be predicted to be unchanged if these clones were expressed as different isotypes/formats.

32.1 Production of human iPSC-derived cerebral cortex neurons: See Example 1.1.

32.2 Generation and labelling of tau species: See Examples 24.2, 24.3. Note that P301S tau was used for both monomeric and aggregated tau preparations.

32.3 Quantification of tau uptake by human iPSC-derived cortical neurons: See Example 24.4.

### Example 33. Affinity Matured Variants of Antibody #44_VH4VK4 Inhibit Uptake of Monomeric and Aggregated Tau into Human Astrocytes

As described in Examples 17 and 25, astrocytic uptake of extracellular tau is proposed to play a role in the pathogenic spreading of tau observed in tauopathies such as Alzheimer's disease. Anti-tau rabbit IgG targeting SEQ ID NO: 1, including antibody clone #44 (Clone 2) and humanised variants of this clone (including antibody #44_VH4VK4) were able to reduce uptake of tau species containing this epitope by human astrocytes (Example 17). Antibodies exhibiting this activity are predicted to be therapeutically useful.

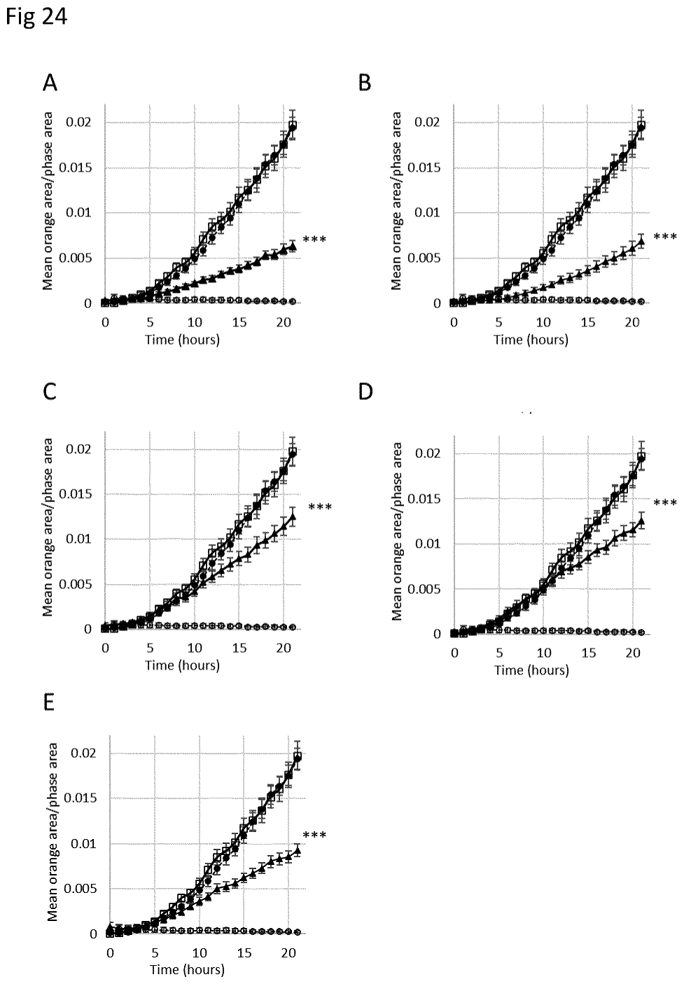

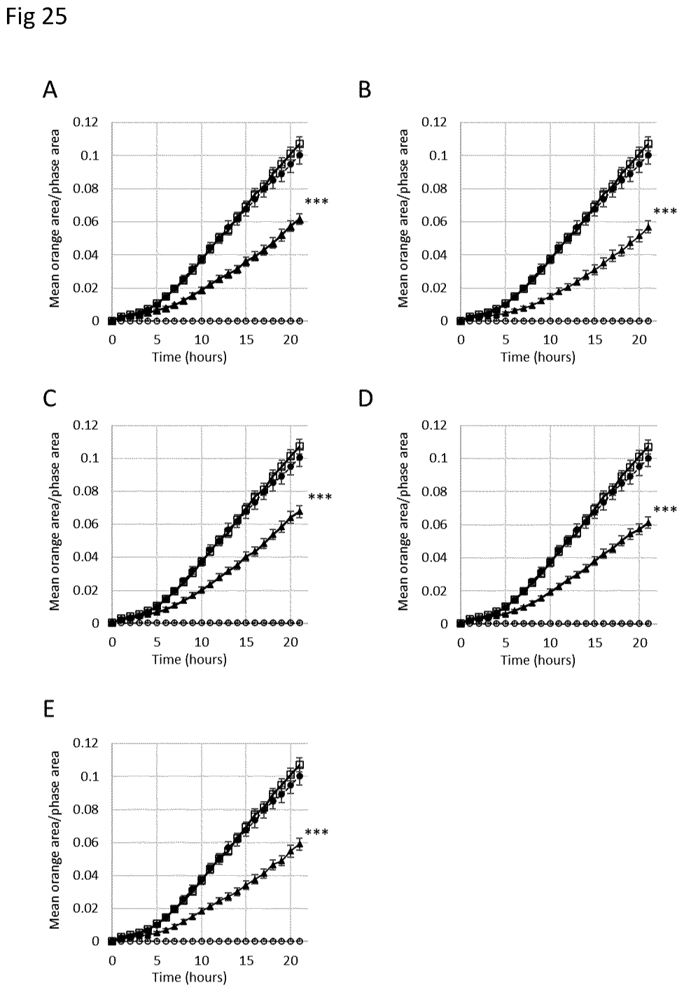

All affinity matured humanised variants of clone #44_VH4VK4 tested, significantly (P<0.001) inhibited uptake of monomeric tau into human astrocytes (FIG. 24): by 67.5±2.5% (H01/pVL), 64.7±3.5% (H02/pVL), 35.6±4.4% (H04/pVL), 35.1±4.3% (H06/pVL), compared to 52.3±3.4% (#44_VH4VK4). All humanised variants tested, also significantly (P<0.001) inhibit uptake of aggregated tau into human astrocytes (FIG. 25) to a similar extent to the rabbit clone: by 38.6±3.2% (H01/pVL), 43.3±3.1% (H02/pVL), 32.6±3.0% (H04/pVL), 39.1±2.9% (H06/pVL), compared to 41.3±2.9% (#44_VH4VK4). Isotype control human IgG1 (anti-fluorescein [Apr. 4, 2020 (enhanced)], Absolute Antibody, Oxford, UK) had no significant effect on tau uptake in this system (inhibition of −1.7±6.3% and −6.7±3.5% for monomeric and aggregated tau respectively). The magnitude of inhibition achieved by affinity matured antibodies was similar to the parental #44_VH4VK4 clone, when compared in a given experiment. It should be noted that some variability in absolute levels of tau uptake and absolute level of antibody-mediated inhibition is expected in these complex cell-based models. This variability is likely due to variability in expression of proteins required for tau uptake, in addition to the usual experimental variability. Data shown in this Example are representative of data observed in multiple experimental runs.

Data demonstrated that, like the parental rabbit antibody (#44, Clone 2), and humanised variants of clone #44, affinity-matured clones based on #44_VH4VK4, targeting epitopes formed by SEQ ID NO: 1 were able to reduce the uptake of tau species containing this epitope (monomeric and multimeric, including full length and truncated forms), by human astrocytes. This activity is predicted to be therapeutically beneficial. Data shown were generated using hIgG1. This activity is CDR-dependent (rather than isotype-dependent) and would therefore be expected to be unchanged if these clones were expressed as different isotypes/formats.

33.1 Production of human iPSC-derived astrocytes: See Example 17.1.

33.2 Generation and labelling of tau species: See Examples 12.2, 12.3. Note that P301S tau was used for both monomeric and aggregated tau preparations.

33.3 Quantification of tau uptake by human iPSC-derived astrocytes: See Example 25.4.

### Example 34. Affinity Matured Variants of Antibody #44_VH4VK4 Detect Increased Levels of High and Low MW Tau Species in Familial Alzheimer's Disease but not Non-Demented Control Postmortem Brain

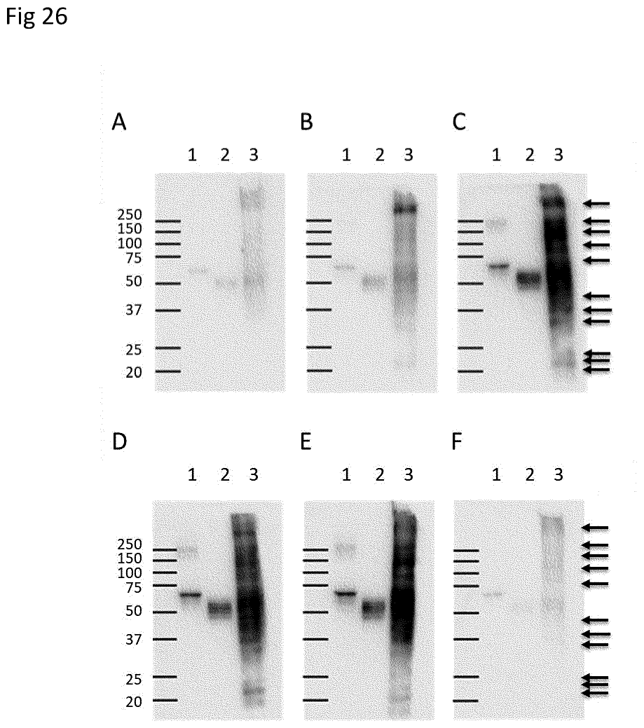

Affinity matured, humanised variants of anti-tau antibody clone #44_VH4VK4 detected disease-relevant forms of tau in postmortem familial Alzheimer's Disease (fAD; Presenilin 1 mutation) cerebral cortex samples but not in non-demented control (NDC) samples. Western blots demonstrate that five variants (all expressed as hIgG1; tested as supernatants from transient transfections): H01/pVL, H02/pVL, H04/pVL, H06/pVL and H16/pVL, detected increased levels of tau in a representative fAD compared to an NDC sample, including multiple high (>75 kD) and low (<40 kD) molecular weight species that are absent in the NDC samples tested (FIG. 26). Blots shown were processed in parallel with identical conditions. Different levels of detection are consistent with the different affinities of these clones for tau, with _H02/pVL, _H04/pVL and_H06/pVL exhibiting the highest affinity for tau and the greatest detection of tau by western blot. Equal quantities of protein were loaded for the NDC and disease brain lysate samples so differences in detection were due to differences in tau species present in disease vs NDC rather than to differences in protein loading. Tau species detected show a similar pattern to the parental humanised antibody (#44_VH4VK4), which in turn, shows similar patterns of detection to the original parental rabbit clone #44 (Example 27, 28), demonstrating that the binding characteristics of the parental rabbit clone #44 have been retained by the humanised affinity matured variants.

34.1 Human brain samples: See Example 8.1.

34.2 Western blot: See Example 27.

### Example 35. Affinity Matured Variant Antibodies, #44_VH4VK4_H04/pVL and #44_VH4VK4_H06/pVL Detect Increased Levels of High and Low MW Tau Species in Familial Alzheimer's Disease, Sporadic Alzheimer's Disease and Dementia with Lewy Bodies, but not Non-Demented Control Postmortem Brain

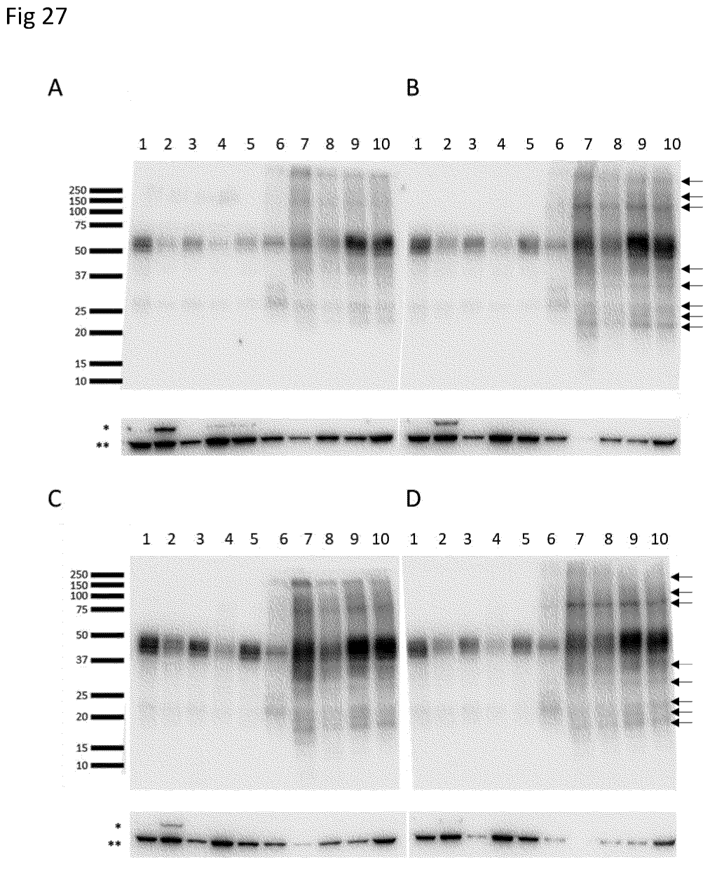

In order to confirm that affinity matured humanised variants of #44_VH4VK4 retained the ability of the parental humanised clone, #44_VH4VK4, to detect disease-relevant tau species across a range of tauopathies and across a panel of patient samples (see Examples 27, 28), four novel affinity matured, humanised antibodies, #44_VH4VK4_H01/pVL, #44_VH4VK4_H02/pVL, #44_VH4VK4_H04/pVL and #44_VH4VK4_H06/pVL (hIgG1) were profiled in more detail. As expected, all affinity matured clones performed similarly to the parental clone #44_VH4VK4 and detected increased levels of both high and low MW species across a panel of familial Alzheimer's disease (fAD) patient samples (FIG. 27). Actin controls demonstrated that the enhanced tau detection in fAD samples is not due to increased protein loading of these samples compared to the non-demented controls. It is noted that actin detection is low in a number of the disease-associated brain lysates. This is likely due to the degeneration of neurons in disease samples, as well as some degree of sample degradation of post-mortem harvested brain samples. Nonetheless, this sample degradation would be predicted to reduce rather than increase the availability of tau (and other proteins) in these samples, so this does not detract from the finding of increased tau detection in disease vs non-disease brain samples.

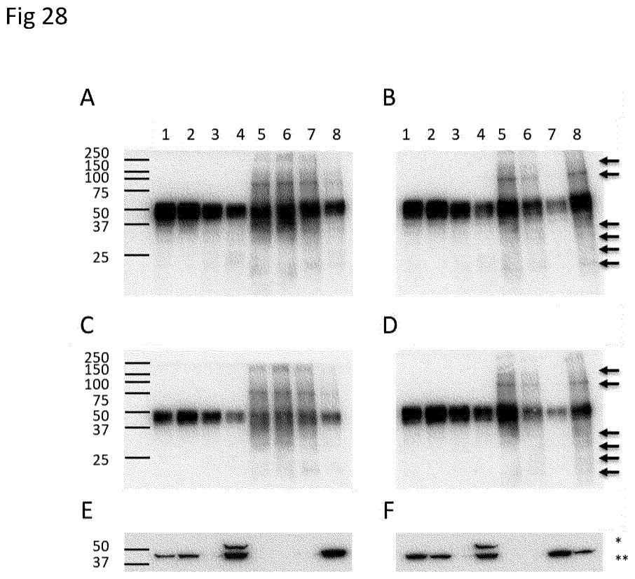

Two clones representing mutations in VH CDR3 (#44_VH4VK4_H04/pVL) and VH CDR2 combined with VH CDR3 (#44_VH4VK4_H06/pVL) were explored further and detected increased levels of high and low MW tau species in sporadic Alzheimer's disease (SAD) and Dementia with Lewy bodies (DLB) brain samples (FIG. 28). Actin and neuronal tubulin controls confirmed that changes in tau levels were not due to variations in protein and/or neuronal levels in the samples tested. As discussed above, actin detection was low in a number of disease-associated samples, but this does not detract from the finding of increased tau detection in disease vs non-disease brain. Data confirmed that affinity matured humanised antibodies based on #44_VH4VK4 detect disease-relevant tau species in a similar manner to that described for the parental #44_VH4VK4 antibody and rabbit IgG clones #44 and #66 (Examples 8, 9, 27, 28). Data support the prediction that the panel of affinity matured humanised variants described in Example 29, as well as affinity matured humanised variants of any other antibody binding to SEQ ID NO: 1, are likely to behave similarly. Binding of #44_VH4VK4_H04 (#44_VH4VK4_H04/pVL (SEQ ID NO: 186 and SEQ ID NO: 160)), #44_VH4VK4_VH06 (#44_VH4VK4_H06/pVL (SEQ ID NO: 188 and SEQ ID NO: 160)), or alternative affinity matured humanised variants, to disease-specific tau species therefore have the potential to be therapeutically useful in the treatment of AD and tauopathy.

35.1 Human brain samples: See Example 8.1 and 9.1.

35.2 Western blot: See Example 27.

### Example 36: Humanised Anti-Tau Antibodies Bind to an Epitope Formed by ₃₇₃THKLTFR₃₇₉ Within Amino Acids 369-381 of 2N4R Tau

Epitope fine mapping was carried out to identify critical residues within the amino acids 369-381 of 2N4R tau; SEQ ID NO: 1 that are required for antibody binding. In a replacement analysis, each residue is mutated to other amino acids to evaluate the importance of the residue for binding to the antibody.

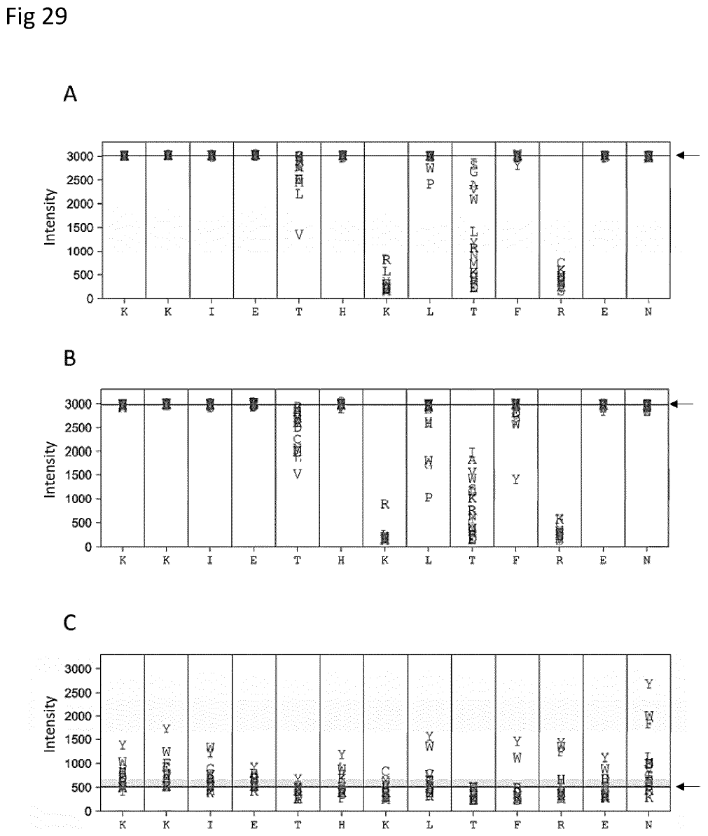

In agreement with data generated with the parental rabbit IgG clone #44 (Clone 2; see Example 20), the replacement analysis shows that amino acid residues in the region, ₃₇₃THKLTFR₃₇₉ are important for binding of affinity matured clones, #44_VH4VK4_H04/pVL (“_H04”) and #44_VH4VK4_H06/pVL (“_H06”) (FIG. 29). In particular, residues, K₃₇₅, T₃₇₇ and R₃₇₉, as substitution of these residues results in a drop of intensity for most substitutes, for both _H04 and _H06. Substitution of K₃₇₅, T₃₇₇ and R₃₇₉, drops signal intensities to background level, suggesting that they are critical for binding of these clones. Substitution of T₃₇₃, L₃₇₆ and F₃₇₈ resulted in reduced intensity for some substitutions suggesting a minor role in antibody binding. Clones _H04 and _H06 are provided as exemplars that reflect affinity matured clones generated based on mutations to the parental VH CDR3 (_H04) and both VH CDR2 and CDR3 (_H06).

Low level binding of the isotype control human IgG1, clearly distinct from the test samples, is detected in this system (FIG. 29), indicating that the ELISA signal obtained for anti-tau antibody clones _H04 and _H06 is CDR-specific.

Data demonstrate that the antibodies described here, exemplified by affinity matured clone _H04 (#44_VH4VK4_H04/pVL (SEQ ID NO: 186 and SEQ ID NO: 160)) and _H06 (#44_VH4VK4_H06/pVL (SEQ ID NO: 188 and SEQ ID NO: 160)) share a common, specific epitope with the parental rabbit IgG, Clone #44 (Clone X) within the peptide sequence (amino acids 369-381 of 2N4R tau; SEQ ID NO: 1); residues K₃₇₅, T₃₇₇ and R₃₇₉ are important for binding of humanised, affinity-matured antibodies generated based on the parental rabbit clone #44 and humanised clone #44_VH4VK4. Slight differences in the effect of substitutions at positions T₃₇₃, H₃₇₄, L₃₇₆ and F₃₇₈ between clones are likely to reflect differences in the affinity of the antibodies tested, as minor interactions are more easily evidenced at lower concentrations (or with lower affinity antibodies).

36.1 Epitope substitution scan analysis-peptide synthesis: see Example 20.1

36.2 Epitope substitution scan analysis-ELISA screening: see Example 20.2

Data are presented as letter plots showing ELISA signal obtained for each peptide tested. Observed deviations from the maximum ELISA signal are indicative of mutations associated with altered (reduced) binding of the tested antibody to the target peptide.

### REFERENCES

Altschul S F, Gish W, Miller W, Myers E W & Lipman D J (1990) Basic local alignment search tool. J. Mol. Biol. 215:405-410
Augustinack J C, Schneider A, Mandelkow E M, Hyman B T (2002) Specific tau phosphorylation sites correlate with severity of neuronal cytopathology in Alzheimer's disease. Acta Neuropathol 103 (1): 26-35
Brownjohn P W, Smith J, Solanki R, Lohmann E, Houlden H, Hardy J, Dietmann S, Livesey F J (2018) Functional studies of missense TREM2 mutations in human stem cell-derived microglia. Stem Cell Rep 10 (4): 1294-1307
Edgar R C. 2004a. MUSCLE: multiple sequence alignment with high accuracy and high throughput. Nucleic Acids Res 32:1792-7.
Edgar R C. 2004b. MUSCLE: a multiple sequence alignment method with reduced time and space complexity. BMC Bioinformatics 5:113.
Evans L D, Wassmer T, Fraser G, Smith J, Perkinton M, Billinton A & Livesey F J (2018) Extracellular monomeric and aggregated tau efficiently enter human neurons through overlapping but distinct pathways. Cell Rep 22 (13): 3612-3624
Guex N & Peitsch M C (1997) SWISS-MODEL and the Swiss-PdbViewer: An environment for comparative protein modelling. Electrophoresis 18, 2714-2723
Hancock D C, O'Reilly N J (2005) Production of polyclonal antibodies in rabbits. Methods Mol Biol 295:27-40
Hu S, Shively L, Raubitschek A, Sherman M, Williams L E, Wong J Y, Shively J E & Wu A M (1996) Minibody: A novel engineered anti-carcinoembryonic antigen antibody fragment (single-chain Fv-CH3) which exhibits rapid, high level targeting of xenografts. Cancer Res 56 (13): 3055-61
Hu N W, Nicoll A J, Zhang D, Mably A J, O'Malley T, Purro S A, Terry C, Collinge J, Walsh D M, Rowan M J (2014) mGlu5 receptors and cellular prion protein mediate amyloid b-facilitated synaptic long-term depression in vivo. Nat Commun 5:3374
Kabat E A & Wu T T (1991) Identical V region amino acid sequences and segments of sequences in antibodies of different specificities. Relative contributions of VH and VL genes, minigenes, and complementarity-determining regions to binding of antibody-combining sites. J Immunol 147 (5): 1709-19
Kontermann R E (2012) Dual targeting strategies with bispecific antibodies. Mabs 4 (2): 182-97
Lefranc M P, Pommié C, Kaas Q, Duprat E, Bosc N, Guiraudou D, Jean C, Ruiz M, Da Piédade I, Rouard M, Foulquier E, Thouvenin V, Lefranc G (2005) IMGT unique numbering for immunoglobulin and T cell receptor constant domains and Ig superfamily C-like domains. Dev Comp Immunol 29 (3): 185-203
Lo M C, Aulabaugh A, Jin G, Cowling R, Bard J, Malamas M, Ellestad G (2004) Evaluation of fluorescence-based thermal shift assays for hit identification in drug discovery. Analytical Biochem 332 (1): 153-9
Martini-Stoica H, Cole A L, Swartzlander D B, Chen F, Wan Y-W, Bajaj L, Bader D A, Lee V M Y, Trojanowski J Q, Liu Z, Sardiello M, Zheng H (2018) TFEB enhances astroglial uptake of extracellular species and reduces tau spreading. J Exp Med 215 (9): 2355-2377
Moore S, Evans L D B, Andersson T, Portelius E, Smith J, Dias T B, Saurat N, McGlade A, Kirwan P, Blennow K, Hardy J, Zetterberg H, Livesey F J (2015) APP metabolism regulates tau proteostasis in human cerebral cortex neurons. Cell Reports 11 (5): 689-96
Morris M, Knudsen Gm, Maeda S, Trinidad J C, Ioanoviciu A, Burlingame A L & Mucke L (2015) Tau post-translational modifications in wild-type and human amyloid precursor protein transgenic mice. Nature Neurosci 18:1183-1189
Pearson W R & Lipman D J (1988) Improved tools for biological sequence comparison. Proc Natl Acad Sci USA 85:2444-2448
Pedersen J T & Sigurdsson E M (2015) Tau immunotherapy for Alzheimer's disease. Trends Mol Med 21 (6): 394-402
Perry L C, Jones T D, Baker M P (2008) New approaches to prediction of immune responses to therapeutic proteins during preclinical development. Drugs R D 9 (6): 385-96
Rauch J N, Luna G, Guzman E, Audouard M, Challis C, Sibih Y E, Leshuk C, Hernandez I, Wegmann S, Hyman B T, Gradinaru V, Kampmann M, Kosik K S (2020) LRP1 is a master regulator of tau uptake and spread. Nature 580(7803):381-385
Roberts M, Sevastou I, Imaizumi Y, Mistry K, Talma S, Dey M, Gartlon J, Ochiai H, Zhou Z, Akasofu S, Tokuhara N, Ogo M, Aoyama M, Aoyagi H, Strand K, Sajedi E, Agarwala K L, Spidel J, Albone E, Horie K, Staddon J M, deSilva R (2020) Pre-clinical characterisation of E2814, a high-affinity antibody targeting the microtubule-binding repeat domain of tau for passive immunotherapy in Alzheimer's disease. Acta Neuropathologica Comms 8:13
Sandusky-Beltran L A, Sigurdsson E M (2020) Tau Immunotherapies: lessons Learned, Current Status and Future Considerations. Neuropharmacol. 175:108104
Shi Y, Kirwan P, Smith J, Robinson H P C, Livesey F J (2012a) Human cerebral cortex development from pluripotent stem cells to functional excitatory synapses. Nature Neurosci 15(3): 477-86
Shi Y, Kirwan P, Livesey F J (2012b) Directed differentiation of human pluripotent stem cells to cerebral cortex neurons and neural networks. Nature Protocols 7 (10): 1836-46
Shi Y, Kirwan P, Smith J, MacLean G, Orkin S H, Livesey F J (2012c) A human stem cell model of early Alzheimer's disease pathology in Down Syndrome. Science Trans Med 4 (124): 124ra29
Sidoryk-Węgrzynowicz M & Strużyńska L (2019) Astroglial contribution to tau-dependent neurodegeneration. Biochem J 476 (22): 3493-3504
Smith T F & Waterman M S (1981) Identification of common molecular subsequences. J. Mol Biol. 147:195-197
Spiess C, Zhai Q & Carter P J (2015) Alternative molecular formats and therapeutic applications for bispecific antibodies. Mol Immunol 67 (2 Pt A): 95-106
Sposito T, Preza E, Mahoney C J, Seto-Salvia N, Ryan N S, Morris H R, Arber C, Devine M J, Houlden H, Warner T T, Bushell T J, Zagnoni M, Kunath T, Livesey F J, Fox N C, Rossor M N, Hardy J, Wray S (2015) Developmental regulation of tau splicing is disrupted in stem cell-derived neurons from frontotemporal dementia patients with the 10+16 splice-site mutation in MAPT. Hum Mol Genet 24 (18): 5260-5269

### SEQUENCE LISTING

The sequence listing submitted herewith forms part of the specification as filed.

1. A humanised antibody or antigen-binding fragment thereof, comprising a sequence that binds specifically to an epitope formed by residues of the amino acid sequence 369-381 (SEQ ID NO: 1) of human 2N4R (amino acids 1-441) tau (SEQ ID NO: 2).

2. The humanised antibody or antigen-binding fragment thereof, according to claim 1, wherein the epitope is formed by residues of the amino acid sequence 373 to 379 (THKLTFR, SEQ ID NO: 150) of human 2N4R (amino acids 1-441) tau (SEQ ID NO: 2).

3. The humanised antibody or antigen-binding fragment thereof, of claim 1, wherein the antigen-binding site comprises human framework sequences (FW1 to FW4) and complementarity-determining region (CDR) sequences selected from SEQ ID NO: 20; SEQ ID NO: 21 or a variant wherein: amino acid 51 is selected from C, V and A; amino acid 54 is selected from R, A and S; amino acid 55 is selected from R, A and V; and amino acid 57 is selected from G, H, N, R, A and S; SEQ ID NO: 22 or a variant wherein: amino acid 96 is selected from S, V, R and A; amino acid 98 is selected from A, S, D, H and T; amino acid 102 is selected from P, V and Y; SEQ ID NO: 23, SEQ ID NO: 24 and SEQ ID NO: 25 or SEQ ID NO 207.

4. The humanised antibody or antigen-binding fragment thereof, of claim 1, wherein the antigen-binding site comprises human framework sequences (FW1 to FW4) and CDRs (HCDR1, HCRD2, HCDR3, LCDR1, LCDR2 and LCDR3, respectively) selected from:
(a) SEQ ID NO: 20, SEQ ID NO: 21, SEQ ID NO: 164, SEQ ID NO: 23, SEQ ID NO: 24 and SEQ ID NO: 25 (VH4_H04);
(b) SEQ ID NO: 20, SEQ ID NO: 21, SEQ ID NO: 162, SEQ ID NO: 23, SEQ ID NO: 24 and SEQ ID NO: 25 (VH4_H02);
(c) SEQ ID NO: 20, SEQ ID NO: 167, SEQ ID NO: 166, SEQ ID NO: 23, SEQ ID NO: 24 and SEQ ID NO: 25 (VH4_H06);
(d) SEQ ID NO: 20, SEQ ID NO: 21, SEQ ID NO: 161, SEQ ID NO: 23, SEQ ID NO: 24 and SEQ ID NO: 25 (VH4_H01);
(e) SEQ ID NO: 20, SEQ ID NO: 167, SEQ ID NO: 164, SEQ ID NO: 23, SEQ ID NO: 24 and SEQ ID NO: 25 (VH4_H06_H04);
(f) SEQ ID NO: 20, SEQ ID NO: 21, SEQ ID NO: 22, SEQ ID NO: 23, SEQ ID NO: 24 and SEQ ID NO: 25 (VH4VK4);
(g) SEQ ID NO: 20, SEQ ID NO: 21, SEQ ID NO: 163, SEQ ID NO: 23, SEQ ID NO: 24 and SEQ ID NO: 25 (VH4_H03);
(h) SEQ ID NO: 20, SEQ ID NO: 21, SEQ ID NO: 165, SEQ ID NO: 23, SEQ ID NO: 24 and SEQ ID NO: 25 (VH4_H05);
(i) SEQ ID NO: 20, SEQ ID NO: 168, SEQ ID NO: 22, SEQ ID NO: 23, SEQ ID NO: 24 and SEQ ID NO: 25 (VH4_H07);
(j) SEQ ID NO: 20, SEQ ID NO: 169, SEQ ID NO: 22, SEQ ID NO: 23, SEQ ID NO: 24 and SEQ ID NO: 25 (VH4_H08);
(k) SEQ ID NO: 20, SEQ ID NO: 170, SEQ ID NO: 22, SEQ ID NO: 23, SEQ ID NO: 24 and SEQ ID NO: 25 (VH4_H09);
(l) SEQ ID NO: 20, SEQ ID NO: 171, SEQ ID NO: 22, SEQ ID NO: 23, SEQ ID NO: 24 and SEQ ID NO: 25 (VH4_H10);
(m) SEQ ID NO: 20, SEQ ID NO: 172, SEQ ID NO: 22, SEQ ID NO: 23, SEQ ID NO: 24 and SEQ ID NO: 25 (VH4_H11);
(n) SEQ ID NO: 20, SEQ ID NO: 173, SEQ ID NO: 22, SEQ ID NO: 23, SEQ ID NO: 24 and SEQ ID NO: 25 (VH4_H12);
(o) SEQ ID NO: 20, SEQ ID NO: 174, SEQ ID NO: 22, SEQ ID NO: 23, SEQ ID NO: 24 and SEQ ID NO: 25 (VH4_H13);
(p) SEQ ID NO: 20, SEQ ID NO: 175, SEQ ID NO: 22, SEQ ID NO: 23, SEQ ID NO: 24 and SEQ ID NO: 25 (VH4_H14);
(q) SEQ ID NO: 20, SEQ ID NO: 176, SEQ ID NO: 22, SEQ ID NO: 23, SEQ ID NO: 24 and SEQ ID NO: 25 (VH4_H15);
(r) SEQ ID NO: 20, SEQ ID NO: 177, SEQ ID NO: 22, SEQ ID NO: 23, SEQ ID NO: 24 and SEQ ID NO: 25 (VH4_H16);
(s) SEQ ID NO: 20, SEQ ID NO: 178, SEQ ID NO: 22, SEQ ID NO: 23, SEQ ID NO: 24 and SEQ ID NO: 25 (VH4_H17);
(t) SEQ ID NO: 20, SEQ ID NO: 179, SEQ ID NO: 22, SEQ ID NO: 23, SEQ ID NO: 24 and SEQ ID NO: 25 (VH4_H18);
(u) SEQ ID NO: 20, SEQ ID NO: 180, SEQ ID NO: 22, SEQ ID NO: 23, SEQ ID NO: 24 and SEQ ID NO: 25 (VH4_H19);
(v) SEQ ID NO: 20, SEQ ID NO: 181, SEQ ID NO: 22, SEQ ID NO: 23, SEQ ID NO: 24 and SEQ ID NO: 25 (VH4_H20);
(w) SEQ ID NO: 20, SEQ ID NO: 167, SEQ ID NO: 161, SEQ ID NO: 23, SEQ ID NO: 24 and SEQ ID NO: 25 (VH4H_06_H01);
(x) SEQ ID NO: 20, SEQ ID NO: 167, SEQ ID NO: 162, SEQ ID NO: 23, SEQ ID NO: 24 and SEQ ID NO: 25 (VH4_H06_H02);
(y) SEQ ID NO: 20, SEQ ID NO: 177, SEQ ID NO: 164, SEQ ID NO: 23, SEQ ID NO: 24 and SEQ ID NO: 25 (VH4_H16_H04);
(z) SEQ ID NO: 20, SEQ ID NO: 21, SEQ ID NO: 22, SEQ ID NO: 23, SEQ ID NO: 24 and SEQ ID NO: 182 (VK4_L2);
(aa) SEQ ID NO: 20, SEQ ID NO: 167, SEQ ID NO: 166, SEQ ID NO: 23, SEQ ID NO: 24 and SEQ ID NO: 182 (VH4_H06/L2);
(bb) SEQ ID NO: 20, SEQ ID NO: 168, SEQ ID NO: 22, SEQ ID NO: 23, SEQ ID NO: 24 and SEQ ID NO: 182 (VH4_H07/L2);
(cc) SEQ ID NO: 20, SEQ ID NO: 170, SEQ ID NO: 22, SEQ ID NO: 23, SEQ ID NO: 24 and SEQ ID NO: 182 (VH4_H09/L2);
(dd) SEQ ID NO: 20, SEQ ID NO: 174, SEQ ID NO: 22, SEQ ID NO: 23, SEQ ID NO: 24 and SEQ ID NO: 182 (VH4_H13/L2); and
(ee) SEQ ID NO: 20, SEQ ID NO: 15, SEQ ID NO: 22, SEQ ID NO: 23, SEQ ID NO: 24 and SEQ ID NO: 182 (VH4_H14/L2);
wherein the sequences are defined according to Kabat nomenclature.

5. The humanised antibody or antigen-binding fragment thereof, of claim 1, wherein the antigen-binding site comprises the VH and/or VL domain sequence of, or a VH and/or VL domain sequence with at least 90, 91, 92, 93, 94, 95, 96, 97, 98 or 99% identity to, a clone selected from:
(a) Clone VH4_H04 of VH SEQ ID NO: 186 and VL SEQ ID NO: 160, respectively;
(b) Clone VH4_H02 of VH SEQ ID NO: 184 and VL SEQ ID NO: 160, respectively;
(c) Clone VH4_H06 of VH SEQ ID NO: 188 and VL SEQ ID NO: 160, respectively;
(d) Clone VH4_H01 of VH SEQ ID NO: 183 and VL SEQ ID NO: 160, respectively;
(c) Clone VH4_H06_H04 of VH SEQ ID NO: 205 and VL SEQ ID NO: 160, respectively;
(f) Clone VH4VK4 (E) of VH SEQ ID NO: 154 and VL SEQ ID NO: 160, respectively;
(g) Clone VH4_H03 of VH SEQ ID NO: 185 and VL SEQ ID NO: 160, respectively;
(h) Clone VH4_H05 of VH SEQ ID NO: 187 and VL SEQ ID NO: 160, respectively;
(i) Clone VH4_H07 of VH SEQ ID NO: 189 and VL SEQ ID NO: 160, respectively;
(j) Clone VH4_H08 of VH SEQ ID NO: 190 and VL SEQ ID NO: 160, respectively;
(k) Clone VH4_H09 of VH SEQ ID NO: 191 and VL SEQ ID NO: 160, respectively;
(l) Clone VH4_H10 of VH SEQ ID NO: 192 and VL SEQ ID NO: 160, respectively;
(m) Clone VH4_H11 of VH SEQ ID NO: 193 and VL SEQ ID NO: 160, respectively;
(n) Clone VH4_H12 of VH SEQ ID NO: 194 and VL SEQ ID NO: 160, respectively;
(o) Clone VH4_H13 of VH SEQ ID NO: 195 and VL SEQ ID NO: 160, respectively;
(p) Clone VH4_H14 of VH SEQ ID NO: 196 and VL SEQ ID NO: 160, respectively;
(q) Clone VH4_H15 of VH SEQ ID NO: 197 and VL SEQ ID NO: 160, respectively;
(r) Clone VH4_H16 of VH SEQ ID NO: 198 and VL SEQ ID NO: 160, respectively;
(s) Clone VH4_H17 of VH SEQ ID NO: 199 and VL SEQ ID NO: 160, respectively;
(t) Clone VH4_H18 of VH SEQ ID NO: 200 and VL SEQ ID NO: 160, respectively;
(u) Clone VH4_H19 of VH SEQ ID NO: 201 and VL SEQ ID NO: 160, respectively;
(v) Clone VH4_H20 of VH SEQ ID NO: 202 and VL SEQ ID NO: 160, respectively;
(w) Clone VH4H_06_H01 of VH SEQ ID NO: 203 and VL SEQ ID NO: 160, respectively;
(x) Clone VH4_H06_H02 of VH SEQ ID NO: 204 and VL SEQ ID NO: 160, respectively;
(y) Clone VH4_H16_H04 of VH SEQ ID NO: 206 and VL SEQ ID NO: 160, respectively;
(z) Clone VK4_L2 of VH SEQ ID NO: 154 and VL SEQ ID NO: 207, respectively;
(aa) Clone VH4_H06/L2 of VH SEQ ID NO: 188 and VL SEQ ID NO: 207, respectively;
(bb) Clone VH4_H07/L2 of VH SEQ ID NO: 189 and VL SEQ ID NO: 207, respectively;
(cc) Clone VH4_H09/L2 of VH SEQ ID NO: 191 and VL SEQ ID NO: 207, respectively
(dd) Clone VH4_H13/L2 of VH SEQ ID NO: 195 and VL SEQ ID NO: 207, respectively;
(cc) Clone VH4_H14/L2 of VH SEQ ID NO: 196 and VL SEQ ID NO: 207, respectively;
(ff) Clone VH3VK3 (A) of SEQ ID NO: 153 and SEQ ID NO: 159, respectively;
(gg) Clone VH3VK4 (B) of SEQ ID NO: 153 and SEQ ID NO: 160, respectively;
(hh) Clone VH4VK2 (C) of SEQ ID NO: 154 and SEQ ID NO: 158, respectively; and
(ii) Clone VH4VK3 (D) of SEQ ID NO: 154 and SEQ ID NO: 159, respectively;
wherein the sequences are defined according to Kabat nomenclature.

6. The humanised antibody or antigen-binding fragment thereof, of claim 1, wherein the antibody comprises the VH and/or VL domain of:
(a) Clone VH4_H04 of VH SEQ ID NO: 186 and VL SEQ ID NO: 160, respectively;
(b) Clone VH4_H02 of VH SEQ ID NO: 184 and VL SEQ ID NO: 160, respectively;
(c) Clone VH4_H06 of VH SEQ ID NO: 188 and VL SEQ ID NO: 160, respectively;
(d) Clone VH4_H01 of VH SEQ ID NO: 183 and VL SEQ ID NO: 160, respectively;
(c) Clone VH4_H06_H04 of VH SEQ ID NO: 205 and VL SEQ ID NO: 160, respectively;
(f) Clone VH4VK4 (E) of VH SEQ ID NO: 154 and VL SEQ ID NO: 160, respectively;
(g) Clone VH4_H03 of VH SEQ ID NO: 185 and VL SEQ ID NO: 160, respectively;
(h) Clone VH4_H05 of VH SEQ ID NO: 187 and VL SEQ ID NO: 160, respectively;
(i) Clone VH4_H07 of VH SEQ ID NO: 189 and VL SEQ ID NO: 160, respectively;
(j) Clone VH4_H08 of VH SEQ ID NO: 190 and VL SEQ ID NO: 160, respectively;
(k) Clone VH4_H09 of VH SEQ ID NO: 191 and VL SEQ ID NO: 160, respectively;
(l) Clone VH4_H10 of VH SEQ ID NO: 192 and VL SEQ ID NO: 160, respectively;
(m) Clone VH4_H11 of VH SEQ ID NO: 193 and VL SEQ ID NO: 160, respectively;
(n) Clone VH4_H12 of VH SEQ ID NO: 194 and VL SEQ ID NO: 160, respectively;
(o) Clone VH4_H13 of VH SEQ ID NO: 195 and VL SEQ ID NO: 160, respectively;
(p) Clone VH4_H14 of VH SEQ ID NO: 196 and VL SEQ ID NO: 160, respectively;
(q) Clone VH4_H15 of VH SEQ ID NO: 197 and VL SEQ ID NO: 160, respectively;
(r) Clone VH4_H16 of VH SEQ ID NO: 198 and VL SEQ ID NO: 160, respectively;
(s) Clone VH4_H17 of VH SEQ ID NO: 199 and VL SEQ ID NO: 160, respectively;
(t) Clone VH4_H18 of VH SEQ ID NO: 200 and VL SEQ ID NO: 160, respectively;
(u) Clone VH4_H19 of VH SEQ ID NO: 201 and VL SEQ ID NO: 160, respectively;
(v) Clone VH4_H20 of VH SEQ ID NO: 202 and VL SEQ ID NO: 160, respectively;
(w) Clone VH4H_06_H01 of VH SEQ ID NO: 203 and VL SEQ ID NO: 160, respectively;
(x) Clone VH4_H06_H02 of VH SEQ ID NO: 204 and VL SEQ ID NO: 160, respectively;
(y) Clone VH4_H16_H04 of VH SEQ ID NO: 206 and VL SEQ ID NO: 160, respectively;
(z) Clone VK4_L2 of VH SEQ ID NO: 154 and VL SEQ ID NO: 207, respectively;
(aa) Clone VH4_H06/L2 of VH SEQ ID NO: 188 and VL SEQ ID NO: 207, respectively;
(bb) Clone VH4_H07/L2 of VH SEQ ID NO: 189 and VL SEQ ID NO: 207, respectively;
(cc) Clone VH4_H09/L2 of VH SEQ ID NO: 191 and VL SEQ ID NO: 207, respectively
(dd) Clone VH4_H13/L2 of VH SEQ ID NO: 195 and VL SEQ ID NO: 207, respectively;
(ee) Clone VH4_H14/L2 of VH SEQ ID NO: 196 and VL SEQ ID NO: 207, respectively:
(ff) Clone VH3VK3 (A) of SEQ ID NO: 153 and SEQ ID NO: 159, respectively;
(gg) Clone VH3VK4 (B) of SEQ ID NO: 153 and SEQ ID NO: 160, respectively;
(hh) Clone VH4VK2 (C) of SEQ ID NO: 154 and SEQ ID NO: 158, respectively; and
(ii) Clone VH4VK3 (D) of SEQ ID NO: 154 and SEQ ID NO: 159, respectively;
wherein the sequences are defined according to Kabat nomenclature.

7. The humanised or human antibody or antigen-binding fragment thereof according to claim 1, wherein the antibody binds an epitope formed by residues of the amino acid sequence 373 to 379 (THKLTFR, SEQ ID NO: 150) of human 2N4R (amino acids 1-441) tau (SEQ ID NO: 2).

8. (canceled)

9. An isolated recombinant DNA or RNA sequence comprising a sequence encoding an isolated antibody or antigen-binding fragment thereof, according to claim 1.

10. The isolated recombinant DNA sequence of claim 9 which is a vector.

11. The isolated recombinant DNA sequence of claim 10 which is an expression vector.

12. The isolated recombinant DNA sequence of claim 11 encoding the antibody or antigen-binding fragment thereof, under control of a promoter.

13. The host cell comprising the DNA or RNA sequence according to claim 9.

14. The host cell of claim 13 capable of expressing the isolated antibody or antigen-binding fragment thereof.

15. (canceled)

16. A composition comprising an isolated antibody or antigen-binding fragment thereof, according to claim 1 and a pharmaceutically acceptable diluent.

17. (canceled)

18. A method of treating or preventing tauopathy in a subject in need thereof, comprising administering to the subject an effective amount of an antibody or antigen-binding fragment thereof according to claim 1.

19. The method according to claim 18, wherein the antibody or antigen-binding fragment thereof of is configured for increasing phagocytosis of tau species in human microglia and/or reducing uptake of monomeric and aggregated tau species by human neurons and/or promoting uptake of tau species by human astrocytes and/or preventing uptake of tau species by human astrocytes and/or preventing tau-mediated inhibition of long term potentiation in rodent models.

20. (canceled)

21. (canceled)

22. A diagnostic kit comprising an antibody or antigen-binding fragment thereof of claim 1 and a reagent capable of detecting an immunological (antigen-antibody) complex which contains said isolated recombinant peptide binding molecule, antigen-binding protein or fragment thereof, wherein optionally said isolated recombinant peptide and/or binding molecule, antigen-binding protein or fragment thereof is immobilized on a solid support (e.g., microplate well), and/or wherein optionally said immunological complex which contains said isolated recombinant peptide, binding molecule, antigen-binding protein or fragment thereof is detectable by ELISA or an alternative immunoassay method or by lateral flow.

23. The diagnostic kit according to claim 22, further comprising one or more control standards and/or specimen diluent and/or washing buffer.

24. The method according to claim 18, wherein the tauopathy is selected from the group consisting of Alzheimer's disease, amyotrophic lateral sclerosis/parkinsonism-dementia complex, argyrophilic grains disease, beta-propeller protein associated neurodegeneration (BPAN), British type amyloid angiopathy, cerebral amyloid angiopathy, Creutzfeldt-Jakob disease, dementia pugilistica, diffuse neurofibrillary tangles with calcification, Down's syndrome, chronic traumatic encephalopathy (CTE), corticobasal degeneration (CBD), frontotemporal dementia (FTD), frontotemporal dementia and parkinsonism linked to chromosome 17 (FTDP-17), frontotemporal lobar degeneration, Gerstmann-Straussler-Scheinker disease, Hallervorden-Spatz disease, inclusion body myositis, multiple system atrophy, myotonic dystrophy, Niemann-pick disease type C, non-guamanian motor neuron disease with neurofibrillary tangles, Parkinson's disease, Pick's disease, post-encephalitic parkinsonism, primary age-related tauopathy (PART), prion protein cerebral amyloid angiopathy, progressive subcortical gliosis, progressive supranuclear palsy (PSP), subacute sclerosing panencephalitis, tangle-dominant dementia, globular glial tauopathy, parkinsonism dementia complex of Guam, progressive non-fluent aphasia, multi-infarct dementia, ischemic stroke, traumatic brain injury (TBI) and stroke.

25. The humanised antibody or antigen-binding fragment thereof according to claim 2, wherein the epitope comprises residues: K375, T377 and R379, or wherein the epitope comprises residues T373, K375, T377 and R379.


SEQUENCE LISTING (207 sequences)

SEQ ID NO: 1 - protein, 13 aa, Artificial Sequence
  Peptide sequence corresponding to amino acids 369-381 of 2N4R tau 1-441
KKEITHKLTFREN

SEQ ID NO: 2 - protein, 441 aa, Artificial Sequence
  Full length 2N4R recombinant tau 1-441
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 18 aa, Homo sapiens
QEFEVMEDHAGTYGLGDR

SEQ ID NO: 4 - protein, 10 aa, Homo sapiens
TPPSSGEPPK

SEQ ID NO: 5 - protein, 15 aa, Homo sapiens
SGYSSPGSPGTPGSR

SEQ ID NO: 6 - protein, 15 aa, Homo sapiens
SRTPSLPTPPTREPK

SEQ ID NO: 7 - protein, 13 aa, Homo sapiens
TPSLPTPPTREPK

SEQ ID NO: 8 - protein, 12 aa, Homo sapiens
LQTAPVPMPDLK

SEQ ID NO: 9 - protein, 8 aa, Homo sapiens
IGSTENLK

SEQ ID NO: 10 - protein, 12 aa, Homo sapiens
VQIVYKPVDLSK

SEQ ID NO: 11 - protein, 16 aa, Homo sapiens
IGSLDNITHVPGGGNK

SEQ ID NO: 12 - protein, 11 aa, Homo sapiens
SPVVSGDTSPR

SEQ ID NO: 13 - protein, 14 aa, Artificial Sequence
  Synthetic antigenic peptide with N-terminal cysteine (C) and amino acids 369-381 (KKIETHKLTFREN) of h au 1 - 441 (2N4R tau)
CKKIETHKLTFREN

SEQ ID NO: 14 - protein, 5 aa, Artificial Sequence
  Clone 1 HCDR1
SNAMI

SEQ ID NO: 15 - protein, 16 aa, Artificial Sequence
  Clone 1 HCDR2
NIGTHGTTYYASWAKG

SEQ ID NO: 16 - protein, 3 aa, Artificial Sequence
  Clone 1 HCDR3
GDI

SEQ ID NO: 17 - protein, 13 aa, Artificial Sequence
  Clone 1 LCDR1
QASQSVDDNNNLA

SEQ ID NO: 18 - protein, 7 aa, Artificial Sequence
  Clone 1 LCDR2
EASTLAS

SEQ ID NO: 19 - protein, 13 aa, Artificial Sequence
  Clone 1 LCDR3
LGEFSCSSADCVV

SEQ ID NO: 20 - protein, 5 aa, Artificial Sequence
  Clone 2 HCDR1
SYAMA

SEQ ID NO: 21 - protein, 16 aa, Artificial Sequence
  Clone 2 HCDR2
CIDRRGGTFYASWVKG

SEQ ID NO: 22 - protein, 7 aa, Artificial Sequence
  Clone 2 HCDR3
DSGAFDP

SEQ ID NO: 23 - protein, 12 aa, Artificial Sequence
  Clone 2 LCDR1
QASQSVYDNYLA

SEQ ID NO: 24 - protein, 7 aa, Artificial Sequence
  Clone 2 LCDR2
AASNLAS

SEQ ID NO: 25 - protein, 13 aa, Artificial Sequence
  Clone 2 LCDR3
LGEFSCTTTDCNV

SEQ ID NO: 26 - protein, 5 aa, Artificial Sequence
  Clone 3 HCDR1
SYAVG

SEQ ID NO: 27 - protein, 16 aa, Artificial Sequence
  Clone 3 HCDR2
CIDSRDSKFYASWAKG

SEQ ID NO: 28 - protein, 7 aa, Artificial Sequence
  Clone 3 HCDR3
DSGAFNP

SEQ ID NO: 29 - protein, 12 aa, Artificial Sequence
  Clone 3 LCDR1
QASQSVYDNYLA

SEQ ID NO: 30 - protein, 7 aa, Artificial Sequence
  Clone 3 LCDR2
AVSNLAS

SEQ ID NO: 31 - protein, 13 aa, Artificial Sequence
  Clone 3 LCDR3
LGEFYCSSIDCNA

SEQ ID NO: 32 - protein, 5 aa, Artificial Sequence
  Clone 4 HCDR1
SYAVG

SEQ ID NO: 33 - protein, 16 aa, Artificial Sequence
  Clone 4 HCDR2
CIDGRDSAFYASWAKG

SEQ ID NO: 34 - protein, 7 aa, Artificial Sequence
  Clone 4 HCDR3
DSGAFNP

SEQ ID NO: 35 - protein, 12 aa, Artificial Sequence
  Clone 4 LCDR1
QASQSVYDNYLS

SEQ ID NO: 36 - protein, 7 aa, Artificial Sequence
  Clone 4 LCDR2
AVSNLAS

SEQ ID NO: 37 - protein, 13 aa, Artificial Sequence
  Clone 4 LCDR3
LGEFYCSSIDCNA

SEQ ID NO: 38 - protein, 5 aa, Artificial Sequence
  Clone 5 HCDR1
TYAMA

SEQ ID NO: 39 - protein, 16 aa, Artificial Sequence
  Clone 5 HCDR2
CIDRRGGTFYASWAKG

SEQ ID NO: 40 - protein, 7 aa, Artificial Sequence
  Clone 5 HCDR3
DSGAFDP

SEQ ID NO: 41 - protein, 12 aa, Artificial Sequence
  Clone 5 LCDR1
QASQSVYDNYLA

SEQ ID NO: 42 - protein, 7 aa, Artificial Sequence
  Clone 5 LCDR2
AASNLAS

SEQ ID NO: 43 - protein, 13 aa, Artificial Sequence
  Clone 5 LCDR3
LGEFSCTTTDCNV

SEQ ID NO: 44 - protein, 5 aa, Artificial Sequence
  Clone 6 HCDR1
KNAMI

SEQ ID NO: 45 - protein, 16 aa, Artificial Sequence
  Clone 6 HCDR2
NIGTRGTTYYASWTKG

SEQ ID NO: 46 - protein, 3 aa, Artificial Sequence
  Clone 6 HCDR3
GDI

SEQ ID NO: 47 - protein, 12 aa, Artificial Sequence
  Clone 6 LCDR1
QSSQSVNNNDLA

SEQ ID NO: 48 - protein, 7 aa, Artificial Sequence
  Clone 6 LCDR2
EASTLAS

SEQ ID NO: 49 - protein, 13 aa, Artificial Sequence
  Clone 6 LCDR3
LGEFSCSSADCVA

SEQ ID NO: 50 - protein, 5 aa, Artificial Sequence
  Clone 7 HCDR1
SYAMS

SEQ ID NO: 51 - protein, 16 aa, Artificial Sequence
  Clone 7 HCDR2
CIDSRGSVYYASWAKG

SEQ ID NO: 52 - protein, 7 aa, Artificial Sequence
  Clone 7 HCDR3
DSGAFDP

SEQ ID NO: 53 - protein, 12 aa, Artificial Sequence
  Clone 7 LCDR1
QASQSVYDNYLS

SEQ ID NO: 54 - protein, 7 aa, Artificial Sequence
  Clone 7 LCDR2
AASNLAS

SEQ ID NO: 55 - protein, 13 aa, Artificial Sequence
  Clone 7 LCDR3
LGEFYCSSMDCNA

SEQ ID NO: 56 - protein, 5 aa, Artificial Sequence
  Clone 8 HCDR1
SNAMI

SEQ ID NO: 57 - protein, 16 aa, Artificial Sequence
  Clone 8 HCDR2
NIGTHGTTYYASWAKG

SEQ ID NO: 58 - protein, 3 aa, Artificial Sequence
  Clone 8 HCDR3
GDI

SEQ ID NO: 59 - protein, 13 aa, Artificial Sequence
  Clone 8 LCDR1
QASQSVDNNNNLA

SEQ ID NO: 60 - protein, 7 aa, Artificial Sequence
  Clone 8 LCDR2
EASTLAS

SEQ ID NO: 61 - protein, 13 aa, Artificial Sequence
  Clone 8 LCDR3
LGEFSCSSADCVA

SEQ ID NO: 62 - protein, 5 aa, Artificial Sequence
  Clone 9 HCDR1
SYAVG

SEQ ID NO: 63 - protein, 16 aa, Artificial Sequence
  Clone 9 HCDR2
CIDSHDNTFYASWAKG

SEQ ID NO: 64 - protein, 7 aa, Artificial Sequence
  Clone 9 HCDR3
DSGAFNP

SEQ ID NO: 65 - protein, 12 aa, Artificial Sequence
  Clone 9 LCDR1
QASQSVYDNYLS

SEQ ID NO: 66 - protein, 7 aa, Artificial Sequence
  Clone 9 LCDR2
AVSNLAS

SEQ ID NO: 67 - protein, 13 aa, Artificial Sequence
  Clone 9 LCDR3
LGEFYCSSIDCNA

SEQ ID NO: 68 - protein, 5 aa, Artificial Sequence
  Clone 10 HCDR1
SNAMI

SEQ ID NO: 69 - protein, 16 aa, Artificial Sequence
  Clone 10 HCDR2
NIGTHGTTYYASWSKG

SEQ ID NO: 70 - protein, 3 aa, Artificial Sequence
  Clone 10 HCDR3
GDI

SEQ ID NO: 71 - protein, 13 aa, Artificial Sequence
  Clone 10 LCDR1
QASQSVDNNNNLA

SEQ ID NO: 72 - protein, 7 aa, Artificial Sequence
  Clone 10 LCDR2
EASTLAS

SEQ ID NO: 73 - protein, 13 aa, Artificial Sequence
  Clone 10 LCDR3
LGEFSCSSADCVA

SEQ ID NO: 74 - protein, 5 aa, Artificial Sequence
  Clone 11 HCDR1
NYAMA

SEQ ID NO: 75 - protein, 16 aa, Artificial Sequence
  Clone 11 HCDR2
CIDRRGGTFYASWAKG

SEQ ID NO: 76 - protein, 7 aa, Artificial Sequence
  Clone 11 HCDR3
DSGAFDP

SEQ ID NO: 77 - protein, 12 aa, Artificial Sequence
  Clone 11 LCDR1
QASQSVYDNYLA

SEQ ID NO: 78 - protein, 7 aa, Artificial Sequence
  Clone 11 LCDR2
AASNLAS

SEQ ID NO: 79 - protein, 13 aa, Artificial Sequence
  Clone 11 LCDR3
LGEFSCTTTDCNV

SEQ ID NO: 80 - protein, 5 aa, Artificial Sequence
  Clone 12 HCDR1
SYAVG

SEQ ID NO: 81 - protein, 16 aa, Artificial Sequence
  Clone 12 HCDR2
CIDSHDNTFYASWAKS

SEQ ID NO: 82 - protein, 7 aa, Artificial Sequence
  Clone 12 HCDR3
DSGAFNP

SEQ ID NO: 83 - protein, 12 aa, Artificial Sequence
  Clone 12 LCDR1
QASQSVYDNYLS

SEQ ID NO: 84 - protein, 7 aa, Artificial Sequence
  Clone 12 LCDR2
AVSNLAS

SEQ ID NO: 85 - protein, 13 aa, Artificial Sequence
  Clone 12 LCDR3
LGEFYCSSIDCNA

SEQ ID NO: 86 - protein, 5 aa, Artificial Sequence
  Clone 13 HCDR1
SYAMG

SEQ ID NO: 87 - protein, 16 aa, Artificial Sequence
  Clone 13 HCDR2
CIDRRGATFYASWAKG

SEQ ID NO: 88 - protein, 7 aa, Artificial Sequence
  Clone 13 HCDR3
DSGAFDP

SEQ ID NO: 89 - protein, 12 aa, Artificial Sequence
  Clone 13 LCDR1
QASQSVYDNYLS

SEQ ID NO: 90 - protein, 7 aa, Artificial Sequence
  Clone 13 LCDR2
AASNLAS

SEQ ID NO: 91 - protein, 13 aa, Artificial Sequence
  Clone 13 LCDR3
LGEFSCTTTDCNV

SEQ ID NO: 92 - protein, 5 aa, Artificial Sequence
  Clone 14 HCDR1
SYAMG

SEQ ID NO: 93 - protein, 16 aa, Artificial Sequence
  Clone 14 HCDR2
CIDRRGGTFYASWAKG

SEQ ID NO: 94 - protein, 7 aa, Artificial Sequence
  Clone 14 HCDR3
DSGAFDP

SEQ ID NO: 95 - protein, 12 aa, Artificial Sequence
  Clone 14 LCDR1
QASQSVYDNYLA

SEQ ID NO: 96 - protein, 7 aa, Artificial Sequence
  Clone 14 LCDR2
AASNLAS

SEQ ID NO: 97 - protein, 13 aa, Artificial Sequence
  Clone 14 LCDR3
LGEFSCTTTDCNV

SEQ ID NO: 98 - protein, 5 aa, Artificial Sequence
  Clone 15 HCDR1
SYAMT

SEQ ID NO: 99 - protein, 16 aa, Artificial Sequence
  Clone 15 HCDR2
CIDTGGSAYYASWAKG

SEQ ID NO: 100 - protein, 7 aa, Artificial Sequence
  Clone 15 HCDR3
DTGAFDP

SEQ ID NO: 101 - protein, 12 aa, Artificial Sequence
  Clone 15 LCDR1
QASQSVYDNNLA

SEQ ID NO: 102 - protein, 7 aa, Artificial Sequence
  Clone 15 LCDR2
AASNLPS

SEQ ID NO: 103 - protein, 13 aa, Artificial Sequence
  Clone 15 LCDR3
LGEFSCSSTDCNA

SEQ ID NO: 104 - protein, 5 aa, Artificial Sequence
  Clone 16 HCDR1
SYAVG

SEQ ID NO: 105 - protein, 16 aa, Artificial Sequence
  Clone 16 HCDR2
CIDSRDSAFYASWAKG

SEQ ID NO: 106 - protein, 7 aa, Artificial Sequence
  Clone 16 HCDR3
DSGAFNP

SEQ ID NO: 107 - protein, 12 aa, Artificial Sequence
  Clone 16 LCDR1
QASQSVYDNYLS

SEQ ID NO: 108 - protein, 7 aa, Artificial Sequence
  Clone 16 LCDR2
AVSNLAS

SEQ ID NO: 109 - protein, 13 aa, Artificial Sequence
  Clone 16 LCDR3
LGEFYCSSIDCNA

SEQ ID NO: 110 - protein, 5 aa, Artificial Sequence
  Clone 17 HCDR1
IYAMG

SEQ ID NO: 111 - protein, 16 aa, Artificial Sequence
  Clone 17 HCDR2
CIDRRGATFYATWAKG

SEQ ID NO: 112 - protein, 7 aa, Artificial Sequence
  Clone 17 HCDR3
DSGAFDP

SEQ ID NO: 113 - protein, 12 aa, Artificial Sequence
  Clone 17 LCDR1
QASQSVYDNNLA

SEQ ID NO: 114 - protein, 7 aa, Artificial Sequence
  Clone 17 LCDR2
AASNLAS

SEQ ID NO: 115 - protein, 13 aa, Artificial Sequence
  Clone 17 LCDR3
LGEFSCTTTDCNV

SEQ ID NO: 116 - protein, 131 aa, Artificial Sequence
  Clone 1 VH sequence
LKGVQCQSVEESGGRLVTPGTPLTLTCTVSGIDLSSNAMIWVRQAPGKGLEYIGNIGTHGTTYYASWAKGRFTISKTSTTVDLKMTSPTTEDTATYFCSRGDIWGPGTLVTVSLGQPKAPSVFPLAPCCGD

SEQ ID NO: 117 - protein, 126 aa, Artificial Sequence
  Clone 1 VL sequence
WLPGATFAQVLTQTESPVSAPLRGTVTINCQASQSVDDNNNLAWYQQKPGQPPKLLIYEASTLASGVSSRFKGSGSGTQFTLTISDVQCDDAATYYCLGEFSCSSADCVVFGGGTEVVVKGDPVAP

SEQ ID NO: 118 - protein, 135 aa, Artificial Sequence
  Clone 2 VH sequence
LKGVQCQSVEESGGRLVTPGTPLTLTCTVSGIDLSSYAMAWVRQAPGKGLEWIGCIDRRGGTFYASWVKGRFTISKTSTTVDLKMTSLTTEDTATYFCARDSGAFDPWGPGTLVTVSSGQPKAPSVFPLAPCCGD

SEQ ID NO: 119 - protein, 126 aa, Artificial Sequence
  Clone 2 VL sequence
WLPGATFAQIVMTQTPSSVSAAVGGTVTIKCQASQSVYDNYLAWYQQKPGQPPKLLIHAASNLASRVPSRFKGSGSGTQFTLTISDVQCDDAATYYCLGEFSCTTTDCNVFGGGTEVVVKGDPVAP

SEQ ID NO: 120 - protein, 135 aa, Artificial Sequence
  Clone 3 VH sequence
LKGVQCQSLEESGGRLVTPGTPLTLTCTVSGIDLSSYAVGWVRQAPGKGLEWIGCIDSHDNTFYASWAKSRFTISKTSTTVDLKITSPTTEDTATYFCARDSGAFNPWGPGTLVTVSSGQPKAPSVFPLAPCCGD

SEQ ID NO: 121 - protein, 125 aa, Artificial Sequence
  Clone 3 VL sequence
WLPGATFARVLTQTPSPVSAAVGGTVTINCQASQSVYDNYLSWYQQKPGQPPKLLIHAVSNLASGVPSRFKGSGSGTQFTLTISDVQCDDAATYYCLGEFYCSSIDCNAFGGGTEVVVKGDPVAP

SEQ ID NO: 122 - protein, 135 aa, Artificial Sequence
  Clone 4 VH sequence
LKGVQCQSLEESGGRLVTPGTPLTLTCTVSGIDLSSYAVGWVRQAPGKGLEWIGCIDGRDSAFYASWAKGRFTISKTSTTVDLKITSPTTEDTATYFCARDSGAFNPWGPGTLVTVSSGQPKAPSVFPLAPCCGD

SEQ ID NO: 123 - protein, 125 aa, Artificial Sequence
  Clone 4 VL sequence
WLPGATFARVLTQTPSPVSAAVGGTVTINCQASQSVYDNYLSWYQQKPGQPPKLLIHAVSNLASGVPSRFKGSGSGTQFTLTISDVQCDDAATYSCLGEFYCSSIDCNAFGGGTEVVVKGDPVAP

SEQ ID NO: 124 - protein, 135 aa, Artificial Sequence
  Clone 5 VH sequence
LKGVQCQSVEESGGRLVTPGTPLTLTCTASGIDLSTYAMAWVRQAPGKGLEWIGCIDRRGGTFYASWAKGRFTISKTSTTVDLKMTSLTTEDTATYFCARDSGAFDPWGPGTLVTVSSGQPKAPSVFPLAPCCGD

SEQ ID NO: 125 - protein, 126 aa, Artificial Sequence
  Clone 5 VL sequence
WLPGATFAQIVMTQTPSPVSVAVGGTVTIKCQASQSVYDNYLAWYQQKPGQPPKLLIHAASNLASGVPSRFKGSGSGTEFTLTISDVQCDDAATYYCLGEFSCTTTDCNVFGGGTEVVVKGDPVAP

SEQ ID NO: 126 - protein, 131 aa, Artificial Sequence
  Clone 6 VH sequence
LKGVQCQSVEESGGRLVTPGTPLTLTCTVSGIDLNKNAMIWVRQAPGKGLEYIGNIGTRGTTYYASWTKGRFTISRTSTTVDLKMTSPTTEDTATYFCTRGDIWGPGTLVTVSLGQPKAPSVFPLAPCCGD

SEQ ID NO: 127 - protein, 125 aa, Artificial Sequence
  Clone 6 VL sequence
WLPGATFAQVLTQTPSPVSAPLGGTVTINCQSSQSVNNNDLAWYQQKPGQRPKVLIYEASTLASGVSSRFKGSGSGTQFTLTISDVQCDDAATYYCLGEFSCSSADCVAFGGGTELVVKGDPVAP

SEQ ID NO: 128 - protein, 135 aa, Artificial Sequence
  Clone 7 VH sequence
LKGVQCQSVEESGGRLVTPGTPLTLTCTVSGIDLSSYAMSWVRQAPGKGLEWIGCIDSRGSVYYASWAKGRFTISKTSTTVDLKVTSPTTEDTATYFCARDSGAFDPWGPGTLVTVSSGQPKAPSVFPLAPCCGD

SEQ ID NO: 129 - protein, 125 aa, Artificial Sequence
  Clone 7 VL sequence
APSATFAQIVMTQTPASVSAAVGGTVTISCQASQSVYDNYLSWYQQKPGQPPKLLIVAASNLASGVPSRFKGSGSGTQFTLTISDVQCDDAATYYCLGEFYCSSMDCNAFGGGTEVVVKGDPVAP

SEQ ID NO: 130 - protein, 135 aa, Artificial Sequence
  Clone 8 VH sequence
AVLKGVQCQSMEESGGRLVTPGTPLTLTCTVSGIDLSSNAMIWVRQAPGKGLEYIGNIGTHGTTYYASWAKGRFTISKTSTTVDLKMTSPTTEDTATYFCSRGDIWGPGTLVTVSLGQPKAPSVFPLAPCCGDTP

SEQ ID NO: 131 - protein, 126 aa, Artificial Sequence
  Clone 8 VL sequence
WLPGATFAQVLTQTPSPVSVAVGGTVTINCQASQSVDNNNNLAWYHQKPGQPPKLLIYEASTLASGVSSRFKGSGSGTQFTLTISGVQCDDAATYYCLGEFSCSSADCVAFGGGTEVVVKGDPVAP

SEQ ID NO: 132 - protein, 135 aa, Artificial Sequence
  Clone 9 VH sequence
LKGVQCQSLEESGGRLVTPGTPLTLTCTVSGIDLSSYAVGWVRQAPGKGLEWIGCIDSHDNTFYASWAKGRFTISKTSTTVDLKITSPTTEDTATYFCARDSGAFNPWGPGTLVTVSSGQPKAPSVFPLAPCCGD

SEQ ID NO: 133 - protein, 125 aa, Artificial Sequence; identical to SEQ ID NO: 121
  Clone 9 VL sequence

SEQ ID NO: 134 - protein, 131 aa, Artificial Sequence
  Clone 10 VH sequence
LKGVQCQSVEESGGRLVTPGTPLTLTCTVSGIDLSSNAMIWVRQAPGKGLEYIGNIGTHGTTYYASWSKGRFTISKTSTTVDLKMTSPTTEDTATYFCSRGDIWGPGTLVTVSLGQPKAPSVFPLAPCCGD

SEQ ID NO: 135 - protein, 126 aa, Artificial Sequence
  Clone 10 VL sequence
WLPGATFAQVLTQTASPVSAAVGGTVTIYCQASQSVDNNNNLAWYQQKPGQPPKLLIYEASTLASGVSSRFKGSGSGTQFTLTISDVQCDDAATYYCLGEFSCSSADCVAFGGGTEMVVKGDPVAP

SEQ ID NO: 136 - protein, 135 aa, Artificial Sequence
  Clone 11 VH sequence
LKGVQCQSVEESGGRLVPPGTPLTLTCTVSGIDLNNYAMAWVRQAPGKGLEWIGCIDRRGGTFYASWAKGRFTISKTSTTVDLKMTSLTTEDTATYFCARDSGAFDPWGPGTLVTVSSGQPKAPSVFPLAPCCGD

SEQ ID NO: 137 - protein, 126 aa, Artificial Sequence
  Clone 11 VL sequence
WLPGATFAQIVMTQTPSPVSVAVGGTVTIKCQASQSVYDNYLAWYQQKPGQPPKLLIHAASNLASRVPSRFKGSGSGTQFTLTISDVQCDDAATYYCLGEFSCTTTDCNVFGGGTEVVVKGDPVAP

SEQ ID NO: 138 - protein, 135 aa, Artificial Sequence; identical to SEQ ID NO: 120
  Clone 12 VH sequence

SEQ ID NO: 139 - protein, 125 aa, Artificial Sequence; identical to SEQ ID NO: 121
  Clone 12 VL sequence

SEQ ID NO: 140 - protein, 135 aa, Artificial Sequence
  Clone 13 VH sequence
LKGVQCQSLEESGGRLVTPGGSLTLTCTVSGIDLSSYAMGWVRQAPGKGLEWIGCIDRRGATFYASWAKGRFTISKTSTTVDLKMTSLTTEDTATYFCARDSGAFDPWGPGTLVTVSSGQPKAPSVFPLAPCCGD

SEQ ID NO: 141 - protein, 126 aa, Artificial Sequence
  Clone 13 VL sequence
WLPGATFAQIVMTQTPSPVSAAVGGTVTIKCQASQSVYDNYLSWYHQKPGQPPKLLIHAASNLASRVPSRFKGSGSGTQFTLTISDVQCDDAATYYCLGEFSCTTTDCNVFGGGTEVVVKGDPVAP

SEQ ID NO: 142 - protein, 135 aa, Artificial Sequence
  Clone 14 VH sequence
LKGVQCQSVEESGGRLVTPGTPLTLTCTVSGIDLNSYAMGWVRQAPGKGLEWIGCIDRRGGTFYASWAKGRFTISKTSTTVDLKMTSLTTEDTATYFCARDSGAFDPWGPGTLVTVSSGQPKAPSVFPLAPCCGD

SEQ ID NO: 143 - protein, 126 aa, Artificial Sequence; identical to SEQ ID NO: 137
  Clone 14 VL sequence

SEQ ID NO: 144 - protein, 119 aa, Artificial Sequence
  Clone 15 VH sequence
QSVEESGGRLVTPGGSLTLTCTVSGIDLSSYAMTWVRQAPGKGLEWIGCIDTGGSAYYASWAKGRFTISKTSTTVALKITSPTTEDTATYFCARDTGAFDPWGPGTLVTVSSGQPKAPS

SEQ ID NO: 145 - protein, 126 aa, Artificial Sequence
  Clone 15 VL sequence
WLPGATFAQIVMTQTPASVSVAVGGTVTINCQASQSVYDNNLAWYQQKPGQPPKLLIVAASNLPSGVPSRFKGSGSGTQFTLTISDVQCDDAATYYCLGEFSCSSTDCNAFGGGTEVVGKGDPVAP

SEQ ID NO: 146 - protein, 135 aa, Artificial Sequence
  Clone 16 VH sequence
LKGVQCQSLEESGGRLVTPGTPLTLTCTVSGIDLSSYAVGWVRQAPGKGLEWIGCIDSRDSAFYASWAKGRFTISKTSTTVDLKITSPTTEDTATYFCARDSGAFNPWGPGTLVTVSSGQPKAPSVFPLAPCCGD

SEQ ID NO: 147 - protein, 125 aa, Artificial Sequence
  Clone 16 VL sequence
WLPGATFARVLTQTPSPVSAVVGGTVTINCQASQSVYDNYLSWYQQKPGQPPKLLIHAVSNLASGVPSRFKGSGSGTQFTLTISDVQCDDAATYYCLGEFYCSSIDCNAFGGGTEVVVKGDPVAP

SEQ ID NO: 148 - protein, 135 aa, Artificial Sequence
  Clone 17 VH sequence
LKGVQCQSVEESGGRLVTPGTPLTLTCTVSGIDLSIYAMGWVRQAPGKGLEWIGCIDRRGATFYATWAKGRFTISKTSTTVDLKLTSLTTEDTATYFCARDSGAFDPWGPGTLVTVSSGQPKAPSVFPLAPCCGD

SEQ ID NO: 149 - protein, 126 aa, Artificial Sequence
  Clone 17 VL sequence
WLPGATFAQIVMTQTPSPVSAAVGGTVTIKCQASQSVYDNNLAWYQQKPGQPPKLLIHAASNLASRVPSRFSGSGSGTQFTLTISDVQCDDAATYYCLGEFSCTTTDCNVFGGGTEVVVKGDPVAP

SEQ ID NO: 150 - protein, 7 aa, Artificial Sequence
  Residues 373 to 379 of human 2N4R (amino acids 1-441) tau (SEQ ID NO 2)
THKLTFR

SEQ ID NO: 151 - protein, 112 aa, Artificial Sequence
  >#44_VH1 humanised
QSVQESGGGLVKPSETLSLTCTVSGIDLSSYAMAWVRQAPGKGLEWIGCIDRRGGTFYASWVKGRFTISKTSTTVDLKMTSLTAEDTATYFCARDSGAFDPWGQGTLVTVSS

SEQ ID NO: 152 - protein, 112 aa, Artificial Sequence
  >#44_VH2 humanised
QSVQESGPGLVKPSETLSLTCTVSGIDLSSYAMAWVRQAPGKGLEWIGCIDRRGGTFYASWVKGRFTISKTSTTVDLQMTSLTAEDTATYFCARDSGAFDPWGQGTLVTVSS

SEQ ID NO: 153 - protein, 113 aa, Artificial Sequence
  >#44_VH3 humanised
QVQLQESGPGLVKPSETLSLTCTVSGIDLSSYAMAWVRQAPGKGLEWIGCIDRRGGTFYASWVKGRFTISKTSTTVDLQMTSLTAEDTATYFCARDSGAFDPWGQGTLVTVSS

SEQ ID NO: 154 - protein, 115 aa, Artificial Sequence
  >#44_VH4 humanised
QVQLQESGPGLVKPSETLSLTCTVSGIDLSSYAMAWVRQAPGKGLEWIGCIDRRGGTFYASWVKGRFTISKDTSKTTVDLQMTSLTAEDTATYFCARDSGAFDPWGQGTLVTVSS

SEQ ID NO: 155 - protein, 115 aa, Artificial Sequence
  >#44_VH5 humanised
QVQLQESGPGLVKPSETLSLTCTVSGIDLSSYAMAWVRQAPGKGLEWIGCIDRRGGTFYASWVKGRFTISRDNSKNTLDLQMTSLTAEDTATYYCARDSGAFDPWGQGTLVTVSS

SEQ ID NO: 156 - protein, 115 aa, Artificial Sequence
  >#44_VH6 humanised
QVQLQESGPGLVKPSETLSLTCTVSGIDLSSYAMSWVRQAPGKGLEWVSCIDRRGGTFYASWVKGRFTISRDNSKNTLDLQMTSLTAEDTATYYCARDSGAFDPWGQGTLVTVSS

SEQ ID NO: 157 - protein, 112 aa, Artificial Sequence
  >#44_VK1 humanised
QIVMTQSPSSLSASVGDRVTITCQASQSVYDNYLAWYQQKPGQPPKLLIHAASNLASRVPSRFKGSGSGTEFTLTISSLQCDDFATYYCLGEFSCTTTDCNVFGGGTEVVIK

SEQ ID NO: 158 - protein, 112 aa, Artificial Sequence
  >#44_VK2 humanised
QIVMTQSPSSLSASVGDRVTITCQASQSVYDNYLAWYQQKPGQPPKLLIHAASNLASGVPSRFKGSGSGTEFTLTISSLQPDDFATYYCLGEFSCTTTDCNVFGGGTKVEIK

SEQ ID NO: 159 - protein, 112 aa, Artificial Sequence
  >#44_VK3 humanised
DIQMTQSPSSLSASVGDRVTITCQASQSVYDNYLAWYQQKPGQPPKLLIHAASNLASGVPSRFKGSGSGTEFTLTISSLQPDDFATYYCLGEFSCTTTDCNVFGGGTKVEIK

SEQ ID NO: 160 - protein, 112 aa, Artificial Sequence
  >#44_VK4 humanised
DIQMTQSPSSLSASVGDRVTITCQASQSVYDNYLAWYQQKPGQPPKLLIHAASNLASGVPSRFSGSGSGTEFTLTISSLQPDDFATYYCLGEFSCTTTDCNVFGGGTKVEIK

SEQ ID NO: 161 - protein, 7 aa, Artificial Sequence
  #44_VH4_H01_CDR3
DVGSFDV

SEQ ID NO: 162 - protein, 7 aa, Artificial Sequence
  #44_VH4_H02_CDR3
DRGDFDV

SEQ ID NO: 163 - protein, 7 aa, Artificial Sequence
  #44_VH4_H03_CDR3
DAGAFDV

SEQ ID NO: 164 - protein, 7 aa, Artificial Sequence
  #44_VH4_H04_CDR3
DAGSFHP

SEQ ID NO: 165 - protein, 7 aa, Artificial Sequence
  #44_VH4_H05_CDR3
DAGHFDY

SEQ ID NO: 166 - protein, 7 aa, Artificial Sequence
  #44_VH4_H06_CDR3
DSGTFDP

SEQ ID NO: 167 - protein, 16 aa, Artificial Sequence
  #44_VH4_H06_CDR2
VIDAAGHTFYASWVKG

SEQ ID NO: 168 - protein, 16 aa, Artificial Sequence
  #44_VH4_H07_CDR2
AIDAAGNTFYASWVKG

SEQ ID NO: 169 - protein, 16 aa, Artificial Sequence
  #44_VH4_H08_CDR2
AIDAAGRTFYASWVKG

SEQ ID NO: 170 - protein, 16 aa, Artificial Sequence
  >#44_VH4_H09_CDR2
AIDARGNTFYASWVKG

SEQ ID NO: 171 - protein, 16 aa, Artificial Sequence
  #44_VH4_H10_CDR2
AIDARGRTFYASWVKG

SEQ ID NO: 172 - protein, 16 aa, Artificial Sequence
  #44_VH4_H11_CDR2
AIDARGATFYASWVKG

SEQ ID NO: 173 - protein, 16 aa, Artificial Sequence
  #44_VH4_H12_CDR2
AIDARGSTFYASWVKG

SEQ ID NO: 174 - protein, 16 aa, Artificial Sequence
  #44_VH4_H13_CDR2
AIDSAGNTFYASWVKG

SEQ ID NO: 175 - protein, 16 aa, Artificial Sequence
  #44_VH4_H14_CDR2
AIDSAGSTFYASWVKG

SEQ ID NO: 176 - protein, 16 aa, Artificial Sequence
  #44_VH4_H15_CDR2
AIDSVGNTFYASWVKG

SEQ ID NO: 177 - protein, 16 aa, Artificial Sequence
  #44_VH4_H16_CDR2
AIDRAGNTFYASWVKG

SEQ ID NO: 178 - protein, 16 aa, Artificial Sequence
  #44_VH4_H17_CDR2
AIDRVGNTFYASWVKG

SEQ ID NO: 179 - protein, 16 aa, Artificial Sequence
  #44_VH4_H18_CDR2
AIDAGGNTFYASWVKG

SEQ ID NO: 180 - protein, 16 aa, Artificial Sequence
  #44_VH4_H19_CDR2
AIDSRGATFYASWVKG

SEQ ID NO: 181 - protein, 16 aa, Artificial Sequence
  #44_VH4_H20_CDR2
AIDSRGSTFYASWVKG

SEQ ID NO: 182 - protein, 13 aa, Artificial Sequence
  #44_VK4_L2_CDR3
LGEFSCQDTDCNV

SEQ ID NO: 183 - protein, 115 aa, Artificial Sequence
  #44_VH4_H01
QVQLQESGPGLVKPSETLSLTCTVSGIDLSSYAMAWVRQAPGKGLEWIGCIDRRGGTFYASWVKGRFTISKDTSKTTVDLQMTSLTAEDTATYFCARDVGSFDVWGQGTLVTVSS

SEQ ID NO: 184 - protein, 115 aa, Artificial Sequence
  >#44_VH4_H02
QVQLQESGPGLVKPSETLSLTCTVSGIDLSSYAMAWVRQAPGKGLEWIGCIDRRGGTFYASWVKGRFTISKDTSKTTVDLQMTSLTAEDTATYFCARDRGDFDVWGQGTLVTVSS

SEQ ID NO: 185 - protein, 115 aa, Artificial Sequence
  #44_VH4_H03
QVQLQESGPGLVKPSETLSLTCTVSGIDLSSYAMAWVRQAPGKGLEWIGCIDRRGGTFYASWVKGRFTISKDTSKTTVDLQMTSLTAEDTATYFCARDAGAFDVWGQGTLVTVSS

SEQ ID NO: 186 - protein, 115 aa, Artificial Sequence
  #44_VH4_H04
QVQLQESGPGLVKPSETLSLTCTVSGIDLSSYAMAWVRQAPGKGLEWIGCIDRRGGTFYASWVKGRFTISKDTSKTTVDLQMTSLTAEDTATYFCARDAGSFHPWGQGTLVTVSS

SEQ ID NO: 187 - protein, 115 aa, Artificial Sequence
  #44_VH4_H05
QVQLQESGPGLVKPSETLSLTCTVSGIDLSSYAMAWVRQAPGKGLEWIGCIDRRGGTFYASWVKGRFTISKDTSKTTVDLQMTSLTAEDTATYFCARDAGHFDYWGQGTLVTVSS

SEQ ID NO: 188 - protein, 115 aa, Artificial Sequence
  #44_VH4_H06
QVQLQESGPGLVKPSETLSLTCTVSGIDLSSYAMAWVRQAPGKGLEWIGVIDAAGHTFYASWVKGRFTISKDTSKTTVDLQMTSLTAEDTATYFCARDSGTFDPWGQGTLVTVSS

SEQ ID NO: 189 - protein, 115 aa, Artificial Sequence
  #44_VH4_H07
QVQLQESGPGLVKPSETLSLTCTVSGIDLSSYAMAWVRQAPGKGLEWIGAIDAAGNTFYASWVKGRFTISKDTSKTTVDLQMTSLTAEDTATYFCARDSGAFDPWGQGTLVTVSS

SEQ ID NO: 190 - protein, 115 aa, Artificial Sequence
  #44_VH4_H08
QVQLQESGPGLVKPSETLSLTCTVSGIDLSSYAMAWVRQAPGKGLEWIGAIDAAGRTFYASWVKGRFTISKDTSKTTVDLQMTSLTAEDTATYFCARDSGAFDPWGQGTLVTVSS

SEQ ID NO: 191 - protein, 115 aa, Artificial Sequence
  #44_VH4_H09
QVQLQESGPGLVKPSETLSLTCTVSGIDLSSYAMAWVRQAPGKGLEWIGAIDARGNTFYASWVKGRFTISKDTSKTTVDLQMTSLTAEDTATYFCARDSGAFDPWGQGTLVTVSS

SEQ ID NO: 192 - protein, 115 aa, Artificial Sequence
  #44_VH4_H10
QVQLQESGPGLVKPSETLSLTCTVSGIDLSSYAMAWVRQAPGKGLEWIGAIDARGRTFYASWVKGRFTISKDTSKTTVDLQMTSLTAEDTATYFCARDSGAFDPWGQGTLVTVSS

SEQ ID NO: 193 - protein, 115 aa, Artificial Sequence
  #44_VH4_H11
QVQLQESGPGLVKPSETLSLTCTVSGIDLSSYAMAWVRQAPGKGLEWIGAIDARGATFYASWVKGRFTISKDTSKTTVDLQMTSLTAEDTATYFCARDSGAFDPWGQGTLVTVSS

SEQ ID NO: 194 - protein, 115 aa, Artificial Sequence
  #44_VH4_H12
QVQLQESGPGLVKPSETLSLTCTVSGIDLSSYAMAWVRQAPGKGLEWIGAIDARGSTFYASWVKGRFTISKDTSKTTVDLQMTSLTAEDTATYFCARDSGAFDPWGQGTLVTVSS

SEQ ID NO: 195 - protein, 115 aa, Artificial Sequence
  #44_VH4_H13
QVQLQESGPGLVKPSETLSLTCTVSGIDLSSYAMAWVRQAPGKGLEWIGAIDSAGNTFYASWVKGRFTISKDTSKTTVDLQMTSLTAEDTATYFCARDSGAFDPWGQGTLVTVSS

SEQ ID NO: 196 - protein, 115 aa, Artificial Sequence
  #44_VH4_H14
QVQLQESGPGLVKPSETLSLTCTVSGIDLSSYAMAWVRQAPGKGLEWIGAIDSAGSTFYASWVKGRSTISKDTSKTTVDLQMTSLTAEDTATYFCARDSGAFDPWGQGTLVTVSS

SEQ ID NO: 197 - protein, 115 aa, Artificial Sequence
  #44_VH4_H15
QVQLQESGPGLVKPSETLSLTCTVSGIDLSSYAMAWVRQAPGKGLEWIGAIDSVGNTFYASWVKGRFTISKDTSKTTVDLQMTSLTAEDTATYFCARDSGAFDPWGQGTLVTVSS

SEQ ID NO: 198 - protein, 115 aa, Artificial Sequence
  #44_VH4_H16
QVQLQESGPGLVKPSETLSLTCTVSGIDLSSYAMAWVRQAPGKGLEWIGAIDRAGNTFYASWVKGRFTISKDTSKTTVDLQMTSLTAEDTATYFCARDSGAFDPWGQGTLVTVSS

SEQ ID NO: 199 - protein, 115 aa, Artificial Sequence
  #44_VH4_H17
QVQLQESGPGLVKPSETLSLTCTVSGIDLSSYAMAWVRQAPGKGLEWIGAIDRVGNTFYASWVKGRFTISKDTSKTTVDLQMTSLTAEDTATYFCARDSGAFDPWGQGTLVTVSS

SEQ ID NO: 200 - protein, 115 aa, Artificial Sequence
  #44_VH4_H18
QVQLQESGPGLVKPSETLSLTCTVSGIDLSSYAMAWVRQAPGKGLEWIGAIDAGGNTFYASWVKGRFTISKDTSKTTVDLQMTSLTAEDTATYFCARDSGAFDPWGQGTLVTVSS

SEQ ID NO: 201 - protein, 115 aa, Artificial Sequence
  #44_VH4_H19
QVQLQESGPGLVKPSETLSLTCTVSGIDLSSYAMAWVRQAPGKGLEWIGAIDSRGATFYASWVKGRFTISKDTSKTTVDLQMTSLTAEDTATYFCARDSGAFDPWGQGTLVTVSS

SEQ ID NO: 202 - protein, 115 aa, Artificial Sequence
  #44_VH4_H20
QVQLQESGPGLVKPSETLSLTCTVSGIDLSSYAMAWVRQAPGKGLEWIGAIDSRGSTFYASWVKGRFTISKDTSKTTVDLQMTSLTAEDTATYFCARDSGAFDPWGQGTLVTVSS

SEQ ID NO: 203 - protein, 115 aa, Artificial Sequence
  #44_VH4_H06-H01
QVQLQESGPGLVKPSETLSLTCTVSGIDLSSYAMAWVRQAPGKGLEWIGVIDAAGHTFYASWVKGRFTISKDTSKTTVDLQMTSLTAEDTATYFCARDVGSFDVWGQGTLVTVSS

SEQ ID NO: 204 - protein, 115 aa, Artificial Sequence
  #44_VH4_H06-H02
QVQLQESGPGLVKPSETLSLTCTVSGIDLSSYAMAWVRQAPGKGLEWIGVIDAAGHTFYASWVKGRFTISKDTSKTTVDLQMTSLTAEDTATYFCARDRGDFDVWGQGTLVTVSS

SEQ ID NO: 205 - protein, 115 aa, Artificial Sequence
  #44_VH4_H06-H04
QVQLQESGPGLVKPSETLSLTCTVSGIDLSSYAMAWVRQAPGKGLEWIGVIDAAGHTFYASWVKGRFTISKDTSKTTVDLQMTSLTAEDTATYFCARDAGSFHPWGQGTLVTVSS

SEQ ID NO: 206 - protein, 115 aa, Artificial Sequence
  #44_VH4_H16-H04
QVQLQESGPGLVKPSETLSLTCTVSGIDLSSYAMAWVRQAPGKGLEWIGAIDRAGNTFYASWVKGRFTISKDTSKTTVDLQMTSLTAEDTATYFCARDAGSFHPWGQGTLVTVSS

SEQ ID NO: 207 - protein, 114 aa, Artificial Sequence
  #44_VK4_L2
DIQMTQSPSSLSASVGDRVTITCQASQSVYDNYLAWYQQKPGQPPKLLIHAASNLASGVPSRFSGSGSGTEFTLTISSLQPDDFATYYCLGEFSCQDTDCNVFGGGTKVEIKRE